(19) United States
(12) Patent Application Publication
(10) Pub. No.: US 2024/0041865 A1
(43) Pub. Date: Feb. 8, 2024

(54) COMBINATION THERAPIES OF WDR-5 INHIBITORS AND PD-1 INHIBITORS

(71) Applicant: HUYABIO International, LLC, San Diego, CA (US)

(72) Inventors: Farbod SHOJAEI, San Diego, CA (US); Jill M. RICONO, San Diego, CA (US); Mireille GILLINGS, San Diego, CA (US); Fairooz KABBINAVAR, Woodland Hills, CA (US)

(21) Appl. No.: 18/351,439

(22) Filed: Jul. 12, 2023

Related U.S. Application Data
(60) Provisional application No. 63/389,249, filed on Jul. 14, 2022.

(51) Int. Cl. A61K 31/496; A61K 39/395; A61P 35/00

(52) U.S. Cl. CPC A61K 31/496; A61K 39/3955; A61P 35/00

(57) ABSTRACT

Provided herein are combinations that include a WDR5 inhibitor and a PD-1 inhibitor that are useful for treating cancer, including reducing and/or preventing cancer metastasis. Provided herein also include pharmaceutical compositions for cancer treatment, including a WDR5 inhibitor and a PD-1 inhibitor. The combinations recited herein are also useful for regulating the response of immune cells in a cancer microenvironment.

### CROSS-REFERENCE

This application claims benefit of priority to U.S. Provisional Application No. 63/389,249, filed Jul. 14, 2022, the contents of which are incorporated herein by reference in their entirety.

### FIELD OF THE INVENTION

The present invention relates to combinations of WDR5 inhibitors and PD-1 inhibitors and the use of such combinations in the treatment of cancer.

### BACKGROUND OF THE INVENTION

Cancer is a significant cause of morbidity and mortality worldwide. While the standards of care for many different cancer types have greatly improved over the years, current standards of care still fail to fully meet the need for effective therapies to improve treatment of cancer. Recently, the clinical use of immuno-oncology agents targeting cytotoxic T-lymphocyte-associated protein 4 (CTLA-4) and the programmed cell death receptor-1 (PD-1) and its ligand PD-L1, have resulted in improvements over the standard of care in the treatment of many cancer types. While these checkpoint inhibitors have produced improved clinical responses in some of such cancers, durable clinical responses only occur in approximately 10-45% of patients. Moreover, a significant number of tumors are either resistant or become refractory to treatment. Accordingly, there is a need in the art for new therapies, including, for example, combination therapies for the treatment of cancers.

Use of small molecule inhibitors to interfere with the protein-protein interaction of MLL1 (mixed lineage leukemia) and WDR5 (WD repeat domain 5) is an effective method to inhibit MLL1 enzymatic activity. For example, when WDR5 is knocked out, the level of H3K4me2/3 decreases and the Hox gene expression is downregulated to block the progression of leukemia. MLL1-WDR5 protein-protein interaction inhibitors have been described in WO2019/205687A1 and WO2020/172932A1, each of which is herein incorporated by reference in its entirety.

Provided herein is a method to treat various cancers including, but not limited to leukemia. The method includes the use of a PD-1 inhibitor in combination with a WDR5 inhibitor such that their combination exhibits a synergistic effect and hence provides solutions to the above and other problems in the art.

### BRIEF SUMMARY

Some embodiments described herein provide a method of treating cancer in a subject having a tumor, including administering to the subject a WDR5 inhibitor compound of Formula I, or a pharmaceutically acceptable salt thereof, and a PD-1 inhibitor, or a pharmaceutically acceptable salt thereof, wherein the compound of Formula I has a structure:

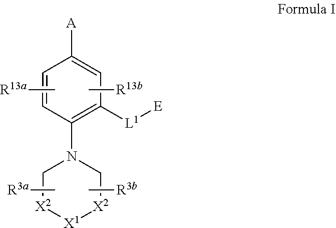

wherein:
A is

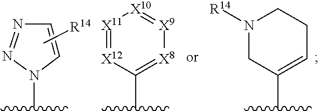

E is

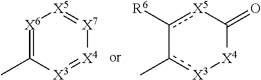

each [symbol] bond is independently a single or double bond;

### L¹ is —NH— or —NH—C(═O)—;

each of R^{3a}, R^{3b}, R^{13a}, R^{13b} is independently hydrogen or a substituent;
X¹ is O, NR¹, or CR^{1a}R^{1b} wherein R¹ is H, C₁-C₆ alkyl, or C₃-C₆ cycloalkyl;
X² is a bond, —CHR^{2a}—, —CHR^{2b}CH₂—, or —CH₂CHR^{2c}—, wherein each of R^{2a}, R^{2b}, and R^{2e} is H, C₁-C₆ alkyl, or C₃-C₆ cycloalkyl;
X³ is N or CR³, or, when the [symbol] bond attached to X³ is a single bond, X³ may also be NR³, wherein R³ is H or a substitutent;
X⁴ is N or CR⁴, wherein R⁴ is H or a substitutent;
X⁵ is N or CR⁵, when the [symbol] bond attached to X⁵ is a single bond, X⁵ may also be NR⁵, wherein R⁵ is H or a substitutent;
X⁶ is N or CR⁶, wherein R⁶ is H or a substitutent;
X⁷ is N or CR⁷, wherein R⁷ is H or a substitutent;
X⁸ is N or CR⁸, wherein R⁸ is H or a substitutent;
X⁹ is N or CR⁹, wherein R⁹ is H or a substitutent;
X¹⁰ is N or CR¹⁰, wherein R¹⁰ is H or a substitutent;
X¹¹ is N or CR¹¹, wherein R¹¹ is H or a substitutent;
X¹² is N or CR¹², where R¹² is H or a substitutent;
each of R^{13a} and R^{13b} is independently H or a substitutent; and
R¹⁴ is H or a substituent.

In some embodiments of the method recited in the immediately preceding paragraph, the compound of Formula I, or a pharmaceutically acceptable salt thereof, and the PD-1 inhibitor may be formulated in separate pharmaceutical formulations; in other embodiments, the compound of Formula I, or a pharmaceutically acceptable salt thereof, and the PD-1 inhibitor may be formulated in the same formulation. Thus, the term “combination” as used herein includes combination therapy with a compound of Formula I, or a pharmaceutically acceptable salt thereof, in a first pharmaceutical composition and PD-1 inhibitor in a second pharmaceutical composition, as well as administration of the compound of Formula I, or a pharmaceutically acceptable salt thereof, and the PD-1 inhibitor in a single pharmaceutical composition. Combination therapy includes administering both the compound of Formula I, or a pharmaceutically acceptable salt thereof, and the PD-1 inhibitor during the same general time frame (e.g., the same day, week, or month) as part of a treatment regimen, taking into account the pharmacokinetic and pharmacodynamic properties of each member of the combination.

In some embodiments, there is provided a method of increasing one or more anti-tumor T cell types in a subject with a tumor, including administering to the subject a combination comprising a compound of Formula I, or a pharmaceutically acceptable salt thereof, and a PD-1 inhibitor, or a pharmaceutically acceptable salt thereof, wherein the compound of Formula I has a structure:

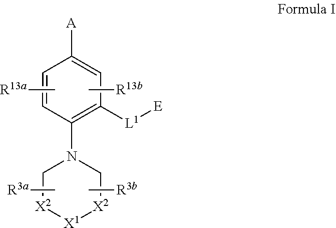

wherein:
A is

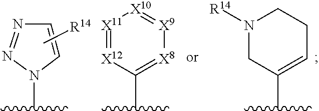

E is

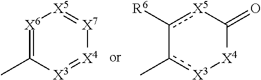

each [symbol] bond is independently a single or double bond;

### L¹ is —NH— or —NH—C(═O)—;

each of R^{3a}, R^{3b}, R^{13a}, R^{13b} is independently hydrogen or a substituent;
X¹ is O, NR¹, or CR^{1a}R^{1b} wherein R¹ is H, C₁-C₆ alkyl, or C₃-C₆ cycloalkyl;
X² is a bond, —CR^{2a}—, —CHR^{2b}CH₂—, or —CH₂CHR^{2c}—, wherein each of R^{2a}, R^{2b}, and R^{2e} is H, C₁-C₆ alkyl, or C₃-C₆ cycloalkyl;
X³ is N or CR³, or, when the [symbol] bond attached to X³ is a single bond, X³ may also be NR³, wherein R³ is H or a substitutent;
X⁴ is N or CR⁴, wherein R⁴ is H or a substitutent;
X⁵ is N or CR⁵, or, when the [symbol] bond attached to X⁵ is a single bond, X⁵ may also be NR⁵, wherein R⁵ is H or a substitutent;
X⁶ is N or CR⁶, wherein R⁶ is H or a substitutent;
X⁷ is N or CR⁷, wherein R⁷ is H or a substitutent;
X⁸ is N or CR⁸, wherein R⁸ is H or a substitutent;
X⁹ is N or CR⁹, wherein R⁹ is H or a substitutent;
X¹⁰ is N or CR¹⁰, wherein R¹⁰ is H or a substitutent;
X¹¹ is N or CR¹¹, wherein R¹¹ is H or a substitutent;
X¹² is N or CR¹², where R¹² is H or a substitutent;
each of R^{13a} and R^{13b} is independently H or a substitutent; and
R¹⁴ is H or a substituent.

Some embodiments described herein comprise a method of inducing at least a two-fold increase in expression of at least one gene associated with anti-tumor T cell infiltration in a tumor in a subject having a tumor, comprising administering to the subject a combination comprising a compound of Formula I, or a pharmaceutically acceptable salt thereof, and a PD-1 inhibitor, or a pharmaceutically acceptable salt thereof, wherein the compound of Formula I has the structure:

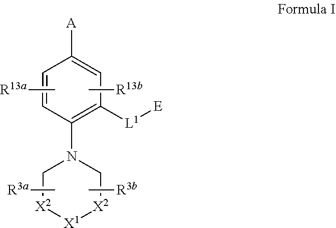

wherein:
A is

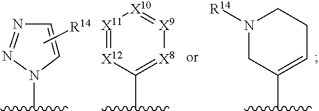

E is

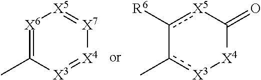

each [symbol] bond is independently a single or double bond;

### L¹ is —NH— or —NH—C(═O)—;

each of R^{3a}, R^{3b}, R^{13a}, R^{13b} is independently hydrogen or a substituent;
X¹ is O, NR¹, or CR^{1a}R^{1b} wherein R¹ is H, C₁-C₆ alkyl, or C₃-C₆ cycloalkyl;
X² is a bond, —CR^{2a}—, —CHR^{2b}CH₂—, or —CH₂CHR^{2c}—, wherein each of R^{2a}, R^{2b}, and R^{2e} is H, C₁-C₆ alkyl, or C₃-C₆ cycloalkyl;
X³ is N or CR³, or, when the [symbol] bond attached to X³ is a single bond, X³ may also be NR³, wherein R³ is H or a substitutent;
X⁴ is N or CR⁴, wherein R⁴ is H or a substitutent;
X⁵ is N or CR⁵ or, when the [symbol] bond attached to X⁵ is a single bond, X⁵ may also be NR⁵, wherein R⁵ is H or a substitutent;
X⁶ is N or CR⁶, wherein R⁶ is H or a substitutent;
X⁷ is N or CR⁷, wherein R⁷ is H or a substitutent;
X⁸ is N or CR⁸, wherein R⁸ is H or a substitutent;
X⁹ is N or CR⁹, wherein R⁹ is H or a substitutent;
X¹⁰ is N or CR¹⁰, wherein R¹⁰ is H or a substitutent;
X¹¹ is N or CR¹¹, wherein R¹¹ is H or a substitutent;
X¹² is N or CR¹², where R¹² is H or a substitutent;
each of R^{13a} and R^{13b} is independently H or a substitutent; and
R¹⁴ is H or a substituent.

In some embodiments of the methods described herein, specifically in the immediately preceding paragraph, the gene associated with an anti tumor T cell is Batf2, Cd274 (PD-L1), DnaselL3, Gbp2, Infg (Inf-γ), IL18 bp, IL24, Lag3, Pdcdl1g2 (PD-L2), Tgtp1, Tnfsf10, or a combination of any two or more thereof.

In some embodiments of the method recited in the foregoing paragraphs of the Brief Summary, the compound of Formula I, or a pharmaceutically acceptable salt thereof, and the PD-1 inhibitor may be formulated in separate pharmaceutical formulations (e.g., oral and parenteral, respectively); in other embodiments, the compound of Formula I, or a pharmaceutically acceptable salt thereof, and the PD-1 inhibitor may be formulated in the same pharmaceutical composition, e.g., as a single unit dose.

In some embodiments described in any of the foregoing paragraphs of the Brief Summary, the PD-1 inhibitor is a small molecule compound, a nucleic acid, a peptide, a protein, an antibody, a peptibody, a diabody, a minibody, a single-chain variable fragment (ScFv), or a fragment or variant thereof. In some embodiments, the PD-1 inhibitor is an antibody, e.g. an anti-PD-1 antibody (or simply “PD-1 antibody”).

In embodiments of the methods described herein, the anti-PD-1 antibody is a monoclonal anti-PD-1 antibody, such as nivolumab, pembrolizumab, pidilizumab, REGN2810 (also known as SAR-439684), PDR001, SHR-1210 or MEDI0680. In some embodiments, the compound of Formula I, or a pharmaceutically acceptable salt thereof, is formulated in a first pharmaceutical composition including the compound of Formula I, or a pharmaceutically acceptable salt, and a first pharmaceutically acceptable excipient, and the PD-1 inhibitor is formulated in a second pharmaceutical composition comprising the PD-1 inhibitor, or a pharmaceutically acceptable salt thereof, and a second pharmaceutically acceptable excipient.

In some embodiments described herein, a kit is provided that includes a combination or a pharmaceutical composition as described herein.

In some embodiments described herein, the combination recited in the methods includes a compound of Formula I, or a pharmaceutically acceptable salt thereof, and an anti-PD-1 antibody in a single pharmaceutical formulation. In other embodiments, the compound of Formula I, or a pharmaceutically acceptable salt thereof, and the anti-PD-1 antibody are formulated as separate pharmaceutical formulations. In some embodiments, the anti-PD-1 antibody comprises one or more of nivolumab, pembrolizumab, pidilizumab, REGN2810 (also known as SAR-439684), PDR001, SHR-1210 and/or MEDI0680. In some embodiments, the combination is formulated for use in any of the methods described herein.

Some embodiments described herein include a method for reducing a level of myeloid-derived suppressor cells (MDSC) in a patient in need thereof by administering a therapeutically effective amount of a combination or pharmaceutical composition described herein.

Some embodiments described herein include a method for reducing a level of regulatory T cells (Treg cells) in a patient in need thereof by administering a therapeutically effective amount of a combination or pharmaceutical composition described herein.

Some embodiments described herein include a method for enhancing the activity of a natural killer (NK) or cytotoxic T-cell activity in-vivo in a subject having cancer (e.g., a cancer patient) by administering a therapeutically effective amount of a combination or pharmaceutical composition described herein.

Some embodiments described herein include a method for enhancing antibody-dependent cell-mediated cytotoxicity in a cancer patient by administering a therapeutically effective amount of a combination or pharmaceutical composition described herein to a subject having a tumor.

Some embodiments described herein include methods for treating diseases, disorders, or alleviating or eliminating the symptoms of diseases and disorders, such as, for example, cancer using a therapeutically effective amount of a combination of a WDR5 inhibitor of Formula I, or a pharmaceutically acceptable salt thereof, and a PD-1 inhibitor administered to a subject in need of treatment and whose cancer has been previously treated with another therapy, such as a checkpoint inhibitor, such as an inhibitor of PD-1, PD-L1, CTLA-4, etc. In some embodiments, the subject may have been treated with a combination of two or more checkpoint inhibitors or with two or more anti-cancer agents, wherein at least one is a checkpoint inhibitor.

In some embodiments, the methods disclosed herein inhibit, reduce or prevent metastasis of a primary tumor in a subject by administering to a subject having a tumor a therapeutically effective amount of a combination or pharmaceutical composition described herein to a subject having a tumor.

The methods described herein include administering a therapeutically effective amount of a combination of a WDR5 inhibitor and PD-1 inhibitor. In some embodiments, the WDR5 inhibitor is one or more compounds of Formula I, or a pharmaceutically acceptable salt thereof. In some embodiments, the PD-1 inhibitor is an anti-PD-1 antibody, e.g., a monoclonal anti-PD-1 antibody.

In some embodiments, the compound of Formula I, or pharmaceutically acceptable slt thereof, is represented by compound of Formula (Ia):

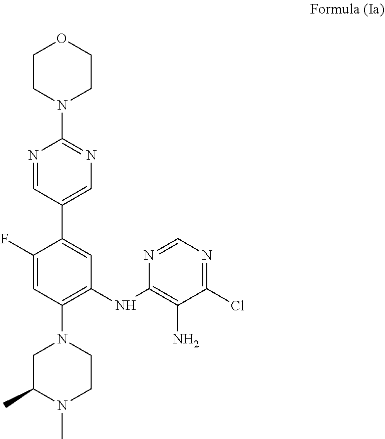 [SMILES: C[C@H]1CN(c2cc(F)c(-c3cnc(N4CCOCC4)nc3)cc2Nc2ncnc(Cl)c2N)CCN1C]

or a pharmaceutically acceptable salt thereof.

In some embodiments, the compound of Formula I, or a pharmaceutically acceptable salt thereof, is represented by compound of Formula (Ib):

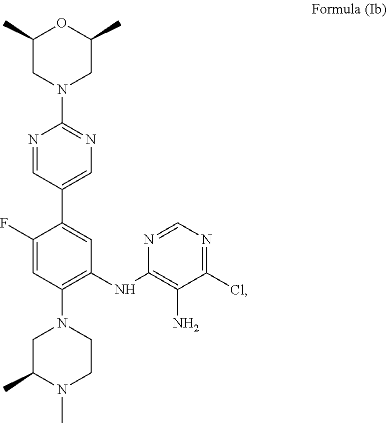 [SMILES: C[C@@H]1CN(c2ncc(-c3cc(Nc4ncnc(Cl)c4N)c(N4CCN(C)[C@@H](C)C4)cc3F)cn2)C[C@H](C)O1]

or a pharmaceutically acceptable salt thereof.

In some embodiments, the compound of Formula I, or a pharmaceutically acceptable salt thereof, is represented by compound of Formula (Ic):

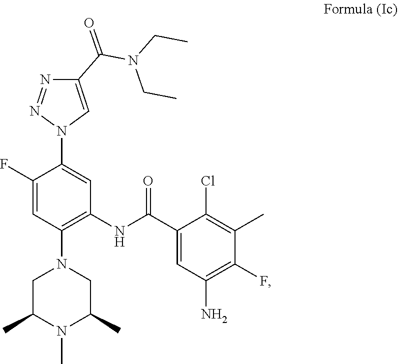 [SMILES: CCN(CC)C(=O)c1cn(-c2cc(NC(=O)c3cc(N)c(F)c(C)c3Cl)c(N3C[C@@H](C)N(C)[C@@H](C)C3)cc2F)nn1]

or a pharmaceutically acceptable salt thereof.

In some embodiments, the compound of Formula I, or a pharmaceutically acceptable salt thereof, is represented by compound of Formula (Id):

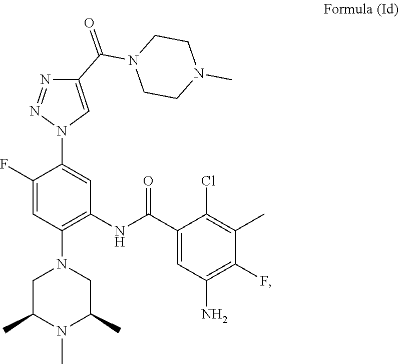 [SMILES: Cc1c(F)c(N)cc(C(=O)Nc2cc(-n3cc(C(=O)N4CCN(C)CC4)nn3)c(F)cc2N2C[C@@H](C)N(C)[C@@H](C)C2)c1Cl]

or a pharmaceutically acceptable salt thereof.

In some embodiments, the compound of Formula I, or a pharmaceutically acceptable salt thereof, is administered in an amount greater than about 80 mg/kg or in a range of about 20 to 120 mg/kg. In some embodiments, the compound of Formula I (or a pharmaceutically acceptable salt thereof) is administered in an amount 20 mg/kg, 30 mg/kg, 40 mg/kg, 50 mg/kg, 60 mg/kg, 70 mg/kg, 80 mg/kg, 90 mg/kg, 100 mg/kg, 110 mg/kg, 120 mg/kg, or a dosage lying within a range of two or more of the foregoing discrete ranges, e.g., 20-50 mg/kg, 40-50 mg/kg, 40-120 mg/kg, etc. (Where dose is represented in terms of mg/kg herein, the numerator represents the mass (mg) of the free base of the compound of Formula I; and the denominator represents the body mass (kg) of the subject/patient.)

In some embodiments, the compound of Formula I (or a pharmaceutically acceptable salt thereof) is administered to a patient in an amount about 1 mg/kg to about 10 mg/kg, preferably about 1.62 mg/kg to 9.72 mg/kg, relative to the body weight of the patient.

In some embodiments, the PD-1 inhibitor is a small molecule compound, a nucleic acid, a peptide, a protein, an antibody, a peptibody, a diabody, a minibody, a single-chain variable fragment (ScFv), or a fragment or variant thereof. In some embodiments, the PD-1 inhibitor is AMP-24, or an antibody, such as a monoclonal antibody, including a human antibody or humanized antibody, such as nivolumab, pembrolizumab, pidilizumab, REGN2810 (also known as SAR-439684), PDR 001, SHR-1210 or MEDI0680. In some embodiments, the PD-1 antibody is administered at an amount of about 1 mg/kg, 2 mg/kg, 3 mg/kg, 5 mg/kg or 10 mg/kg.

In some embodiments, the cancer treated is one or more of: prostate cancer; skin cancer; ovarian cancer; one or more cancers of non-lymphoid parenchymal organs (including the heart, placenta, skeletal muscle and lung); breast cancer; one or more cancers of the head and neck (including various lymphomas, such as mantle cell lymphoma); Non-Hodgkin B cell lymphoma; PTCL,;adenoma; squamous cell carcinoma; laryngeal carcinoma; salivary carcinoma; thymomas and thymic carcinoma; leukemia; one or more cancers of the retina; one or more cancers of the esophagus; multiple myeloma; melanoma; colorectal cancer; lung cancer; cervical cancer; endometrium carcinoma; gallbladder cancer; liver cancer; thyroid follicular cancer; gastric cancer; non-small cell lung carcinoma; glioma; urothelial cancer; bladder cancer; prostate cancer; renal cell cancer; infiltrating ductal carcinoma; and glioblastoma multiform. In some embodiments, the cancer treated is an immune driven cancer such as, breast cancer, bile duct cancer, cervical cancer, colorectal cancer, esophageal cancer, head and neck cancer, brain cancer, kidney cancer, leukemia, liver cancer, lung cancer, lymphoma, melanoma, mesothelioma, multiple myeloma, ovarian cancer, pancreatic cancer, prostrate cancer, sarcoma, skin cancer, stomach cancer, or endometroial cancer.

Some embodiments describe herein include a method for reducing or preventing metastasis using a combination of a WDR5 inhibitor and PD-1 inhibitor, wherein the combination is administered prior to, concurrently with, or subsequent to treatment of the primary tumor, or some combination of prior to, concurrently with, and subsequent to treatment of the primary tumor. Treatment of the primary tumor can include one or more of radiation, surgery, chemotherapy, immunotherapy, targeted therapy, hormone therapy, stem cell transplant, cryotherapy, laser therapy, and precision medicine. The primary tumor can include, without limitation, cancer of the breast, lung, bladder, skin, intestine, colon, kidney, ovary, pancreas, prostate, brain, stomach, thyroid, head and neck, gastroesophageal tract, connective or other nonepithelial tissue, lymphatic cells, or uterus. In some embodiments, the primary tumor is breast cancer that is advance metastatic breast cancer that may be triple negative.

In some embodiments, the WDR5 inhibitor is used alone to prime the tumor for a period of time before treatment using the combination of WDR5 inhibitor and PD-1 inhibitor. The period for priming can be 1 day, 2 days, 3 days, 4 days, 5 days, 6 days, one week, greater than one week, two weeks, greater than two weeks, three weeks, or greater than three weeks. In some embodiments of the priming, the WDR5 inhibitor is administered bi-weekly or tri-weekly for a period of time before treatment using the combination with PD-1 inhibitor begins.

In some embodiments, the disclosure herein incudes kits for (i) use in reducing metastasis of a primary tumor; (ii) preventing or delaying recurrence of the cancer; (iii) extending disease-or tumor free survival time; (iv) increasing overall survival time; (v) reducing the frequency of treatment; (vi) relieving one or more symptoms of the cancer, and (vii) reducing tumor burden. The kits include a WDR5 inhibitor and PD-1 inhibitor. In some embodiments of the kit, the WDR5 inhibitor and PD-1 inhibitor comprise separate formulations. In some embodiments, the PD-1 inhibitor and WDR5 inhibitor are in different containers. The kits can include instructions for use and/or reagents and medical devices for administration.

In some embodiments, the disclosure herein provides methods for treating a subject with a primary cancer with a combination of an WDR5 inhibitor and a PD-1 inhibitor whereby the treatment results in one or more of the following: (i) reduces or slows tumor metastasis; (ii) prevents or delays recurrence of the cancer; (iii) extends disease-or tumor free survival time; (iv) increases overall survival time; (v) reduces the frequency of treatment; (vi) relieves one or more symptoms of the cancer or combinations of the aforementioned, and (vii) reduces tumor burden. In some embodiments of this method the patient has previously been treated with a checkpoint inhibitor, such as a PD-1, a PD-L1, or a CTLA-4 checkpoint inhibitor.

In some embodiments of the methods disclosed herein, the metastasis that is reduced by the treatment methods described herein include metastasis of one or more of the adrenal gland, brain and/or spinal cord, bone, lung, liver and/or pleura, gastrointestinal tract, peritoneum, muscle, lymph nodes and skin.

In some embodiments of the methods disclosed herein are methods for reducing or preventing metastasis in which the primary tumor or secondary tumor of the subject being treated with the combination is a cancer of the breast, lung, bladder, skin, intestine, colon, kidney, ovary, pancreas, prostate, liver, brain, stomach, thyroid, head and neck, gastroesophageal tract, myeloid, lymphoid, connective or other nonepithelial tissue, and uterus. In some embodiments of this method, the cancer is triple negative breast cancer.

In some embodiments of the methods, the methods further comprise treatment of the subject with an E-selectin inhibitor, or plerixafor, or a combination of an E-selectin inhibitor and plerixafor. In some embodiments of this method the E-selectin inhibitor and/or plerixafor is given prior, concurrently, or subsequently, or combinations of prior, concurrently or subsequently, to the WDR5 inhibitor and PD-1 combination.

In some embodiments, the method of treatment further comprises treating the subject with an integrin inhibitor, or an antibody from the group comprising etaracizumab, intetumumab, or abituzumab or a combination of an integrin inhibitor and an antibody from the group comprising etaracizumab, etaracizumab, inteturnumab, or abituzumab. In other aspects, treatment further comprises treating the subject with a matrix metalloproteinase inhibitor, wherein said matrix metalloproteinase inhibitor is given prior, concurrently, or subsequently, or combinations of prior, concurrently or subsequently, to the WDR5 inhibitor and PD-1 inhibitor.

Also provided herein are methods of any of the foregoing paragraphs, wherein the subject has received one or more previous treatments for the cancer. In some embodiments, the subject has previously received immunotherapy. In some embodiments, the subject has previously received immunotherapy with one or more checkpoint inhibitors. In some embodiments, at least one of the one or more checkpoint inhibitors is selected from one or more PD-1 inhibitors, one or more PD-L1 inhibitors, one or more CTLA-4 inhibitors, and combinations of two or more thereof.

One skilled in the art will recognize that, as there are described herein methods of use comprising a combination of a WDR5 inhibitor, or a pharmaceutically acceptable salt thereof, and a PD-1 inhbitor, or a pharmaceutically acceptable salt thereof, also provided herein is the use of the combination recited in any of the foregoing paragraphs, for use in treatment of cancer in a subject in need of such treatment. Also provided is a combination as recited in any of the foregoing paragraphs for treatment of cancer in a subject, as described herein. Additionally, herein is provided a process of making a combination as recited in any of the foregoing paragraphs for use in the treatment of cancer in a subject in need of such treatment, as described in more detail herein.

### DESCRIPTION OF THE FIGURES

FIG. 1A illustrates group tumor growth as median tumor volume (mm³, y-axis) over time (days, x-axis) for the MC38 group described in Example 1 herein.

FIG. 1B illustrates the efficacy of the treatment for the MC38 group as percent body weight change over time (days, x-axis) for the MC38 group described in Example 1 herein.

FIG. 2A illustrates group tumor growth as median tumor volume (mm³, y-axis) over time (days, x-axis) for the E0771 group, described in Example 1 herein.

FIG. 2B illustrates the efficacy of the treatment for the E0771 group as percent body weight change over time (days, x-axis) for the E0771 group, described in Example 1 herein.

FIG. 3A illustrates group tumor growth as median tumor volume (mm³, y-axis) over time (days, x-axis) for the 3LL group, described in Example 1 herein.

FIG. 3B illustrates the efficacy of the treatment for the 3LL group as percent body weight change over time (days, x-axis) for the 3LL group, described in Example 1 herein.

FIGS. 4A-4G illustrate the effect on immune cells of in vivo treatment with vehicle control, IgG control, HBI-2375 (80 mpk), anti-PD-1 antibody (10 mpk; BIW) and HBI-2375 (80 mpk)+anti-PD-1 antibody (10 mpk; BIW).

FIG. 5A illustrates the infiltration of CD8+ T cells in MC38 tumors treated with all treatment groups.

FIG. 5B illustrates the infiltration of CD8+ T cells in 3LL tumors treated with all treatment groups.

FIG. 6 illustrates the fold change of immune related genes in MC38 and 3LL tumors treated with the combination of HBI-2375 and anti-PD-1.

### DETAILED DESCRIPTION

All patents, applications, published applications are incorporated by reference in their entirety. Unless defined otherwise, all technical and scientific terms used herein have the same meaning as commonly understood by those of ordinary skill in the art to which the invention belongs. The chemical structures and formulae set forth herein are constructed according to the standard rules of chemical valency known in the chemical arts.

Should a discrepancy exist between a depicted structure and a name given for that structure, the depicted structure is to be accorded more weight. Where the stereochemistry of a structure or a portion of a structure is not indicated in a depicted structure or a portion of the depicted structure, the depicted structure is to be interpreted as encompassing all of its possible stereoisomers.

Any methods, devices and materials similar or equivalent to those described herein can be used in the practice of this invention. The following definitions are provided to facilitate understanding of certain terms used frequently herein and are not meant to limit the scope of the present disclosure. In the event that there is a plurality of definitions for a term herein, those in this section prevail unless stated otherwise. Headings used herein are for organizational purposes only and in no way limit the invention described herein.

Some embodiments described herein are combinations (e.g., combination therapies, such as therapeutic methods and uses, kits and compositions) for treating cancer. In some embodiments, the combinations described herein comprise a WDR5 inhibitor and a PD-1 inhibitor. In some embodiments, the WDR5 inhibitor is a phenyl triazole derivative. In some embodiments, a combination may comprise a first pharmaceutical composition and a second pharmaceutical composition. In some embodiments, the first pharmaceutical composition comprises a WDR5 inhibitor and the second pharmaceutical composition comprises a PD-1 inhibitor. In some embodiments, the first pharmaceutical composition and the second pharmaceutical composition are co-packaged as a kit, which may further include instructions for co-administration of the first and second pharmaceutical compositions. In some embodiments, the first and second compositions may be packaged separately for combination in a clinical setting by administering them to a patient within a time frame during which the patient derives clinical benefit from the first pharmaceutical composition and the second pharmaceutical composition at the same time. In some embodiments, a combination may comprise a pharmaceutical composition comprising a WDR5 inhibitor and a PD-1 inhibitor. In some embodiments, a combination comprises a unit dosage form of a pharmaceutical composition comprising a WDR5 inhibitor and a PD-1 inhibitor. In some embodiments, a combination comprises a first pharmaceutical composition comprising a WDR5 inhibitor for use in the treatment of cancer in combination with a second pharmaceutical composition comprising a PD-1 inhibitor. In some embodiments, a combination comprises a use of a WDR5 inhibitor for preparation of a first pharmaceutical composition for use in the treatment of cancer in combination with a second pharmaceutical composition comprising a PD-1 inhibitor.

The term “PD-1 inhibitor” refers to a moiety (e.g., compound, nucleic acid, polypeptide, antibody) that decreases, inhibits, blocks, abrogates or interferes with the activity or expression of PD-1 (e.g., Programmed Cell Death Protein 1; PD-1 (CD279); GI: 145559515), including variants, isoforms, species homologs of human PD-1 (e.g., mouse) and analogs that have at least one common epitope with PD-1. A PD-1 inhibitor includes molecules and macromolecules such as, for example, compounds, nucleic acids, polypeptides, antibodies, peptibodies, diabodies, minibodies, single-chain variable fragments (ScFv), and fragments or variants thereof. Thus, a PD-1 inhibitor as used herein refers to any moiety that antagonizes PD-1 activity or expression. PD-1 inhibitor efficacy can be measured, for example, by its inhibitor concentration at 50% (half-maximal inhibitor concentration or IC₅₀). PD-1 inhibitors include exemplary compounds and compositions described herein. An anti-PD-1 antibody (or PD-1 antibody) refers to a PD-1 inhibitor which is a monoclonal or polyclonal antibody as described herein capable of selectively binding to and inhibiting the activity of PD-1.

The terms “nivolumab,” “pembrolizumab,” “pidilizumab,” “AMP-224,” “REGN2810,” “PDR 001,”, “SHR-1210”, “SAR-439684” and “MEDI0680” are used in accordance with their plain and ordinary meaning as understood in the art.

The terms “polypeptide” and “protein” are used interchangeably herein and refer to any molecule that includes at least 2 or more amino acids.

The term “effective amount” refers to the amount of a therapy (e.g., a combination provided herein or another active agent described herein such as an anti-cancer agent described herein) which is sufficient to accomplish a stated purpose or otherwise achieve the effect for which it is administered. An effective amount can be sufficient to reduce and/or ameliorate the progression, development, recurrence, severity and/or duration of a given disease, disorder or condition and/or a symptom related thereto, or can be sufficient to reduce the level of activity of a polypeptide (e.g., PD-1). An effective amount can be a “therapeutically effective amount” which refers to an amount sufficient to provide a therapeutic benefit such as, for example, the reduction or amelioration of the advancement or progression of a given disease, disorder or condition, reduction or amelioration of the recurrence, development or onset of a given disease, disorder or condition, and/or to improve or enhance the prophylactic or therapeutic effect(s) of another therapy. A therapeutically effective amount of a composition described herein can enhance the therapeutic efficacy of another therapeutic agent.

The term “regimen” refers to a protocol for dosing and timing the administration of one or more therapies (e.g., combinations described herein or another active agent such as for example an anti-cancer agent described herein) for treating a disease, disorder, or condition described herein. A regimen can include periods of active administration and periods of rest as known in the art. Active administration periods include administration of combinations and compositions described herein and the duration of time of efficacy of such combinations and compositions. Rest periods of regimens described herein include a period of time in which no compound is actively administered, and in certain instances, includes time periods where the efficacy of such compounds can be minimal. Combination of active administration and rest in regimens described herein can increase the efficacy and/or duration of administration of the combinations and compositions described herein.

The terms “therapies” and “therapy” refer to any protocol(s), method(s), and/or agent(s) that can be used in the prevention, treatment, management, and/or amelioration of a disease, disorder, or condition or one or more symptoms thereof. In certain instances the term refers to other active agents such as anti-cancer agents described herein. The terms “therapy” and “therapies” can refer to anti-viral therapy, anti-bacterial therapy, anti-fungal therapy, anti-cancer therapy, biological therapy, supportive therapy, and/or other therapies useful in treatment, management, prevention, or amelioration of a disease, disorder, or condition or one or more symptoms thereof known to one skilled in the art, for example, a medical professional such as a physician.

The term “patient” or “subject” refers to a mammal, such as a human, bovine, rat, mouse, dog, monkey, ape, goat, sheep, cow, or deer. Generally a patient as described herein is human.

The terms “inhibition,” “inhibit,” and “inhibiting” refer to a reduction in the activity or expression of a polypeptide or reduction or amelioration of a disease, disorder, or condition or a symptom thereof. Inhibiting as used here can include: partially or totally blocking stimulation; decreasing, preventing, or delaying activation; or inactivating, desensitizing, or down-regulating protein or enzyme activity.

Antibodies described herein can be polyclonal or monoclonal and include xenogeneic, allogeneic, or syngeneic forms and modified (e.g., humanized or chimeric) versions thereof, as well as single-domain variants thereof. Antibodies may include polypeptide products of B cells within the immunoglobulin class of polypeptides able to bind to a specific molecular antigen and composed of two identical pairs of polypeptide chains, wherein each pair has one heavy chain (about 50-70 kDa) and one light chain (about 25 kDa) and each amino-terminal portion of each chain includes a variable region of about 100 to about 130 or more amino acids and each carboxy-terminal portion of each chain includes a constant region (See Borrebaeck (ed.) (1995), Antibody Engineering, Second Edition, Oxford University Press.; Kuby (1997) Immunology, Third Edition, W.H. Freeman and Company, New York). Antibodies can also include single-domain antibodies, e.g., those derived from members of the camelids, nanobodies, antibody fragments, VHH antibodies, especially humanized variants thereof. For example, production of anti-PD-1 single-domain antibody, for example a heavy chain single-domain antibody; a chimeric antibody of VHH from a camelid animal and Fc domain of human IgG4; or a humanized antibody, or wherein the VHH is fused to another molecule, for example, a Fe domain of an immunoglobin (for example, IgG), are taught in US Pre-Grant Publicaiton No. US 2021/061912 A1, which is incorporated herein by reference in its entirety.

The term “monoclonal antibody(ies)” refers to a population of antibody molecules that contain one species of an antigen binding site capable of immunoreacting with a particular epitope of an antigen, whereas the term “polyclonal antibody(ies)” refers to a population of antibody molecules that contain multiple species of antigen binding sites capable of interacting with a particular antigen. A monoclonal antibody, typically displays a single binding affinity for a particular antigen with which it immuno-reacts. In some embodiments, the monoclonal antibodies to be used in accordance with the present invention can be made by a variety of techniques, including, for example, the hybridoma method (e.g., Kohler and Milstein., Nature, 256:495-97 (1975); Hongo et al., Hybridoma, 14 (3): 253-260 (1995); Harlow et al., Antibodies: A Laboratory Manual, (Cold Spring Harbor Laboratory Press, 2nd ed. 1988); Hammerling et al., in: Monoclonal Antibodies and T-Cell Hybridomas 563-681 (Elsevier, N.Y., 1981)); recombinant DNA methods (see, e.g., U.S. Pat. No. 4,816,567); phage-display technologies (see, e.g., Clackson et al., Nature, 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004), and technologies for producing human or human like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; and 5,661,016; Markset al., Biotechnology 10: 779-783 (1992); Lon berg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996); and Lonberg and Huszar, Intern. Rev. Immunol. 13: 65-93 (1995).

The monoclonal antibodies herein also include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is(are) identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, pp. 6851-6855 (1984)). “Humanized antibody(ies)” can be considered as a subset of chimeric antibodies described herein.

The term “human” when used in reference to an antibody or a functional fragment thereof (e.g., “humanized antibody(ies)”) refers to an antibody or functional fragment thereof that has a human variable region or a portion thereof corresponding to human germline immunoglobulin sequences. Such human germline immunoglobulin sequences are described by Kabat et al. (1991) Sequences of Proteins of Immunological Interest, Fifth Edition, U.S. Department of Health and Human Services, NIH Publication No. 91-3242. A human antibody in the context of the present invention, can include an antibody that binds to PD-1 or variants thereof as described herein, including a single-domain antibody, a nanobody, a camelid antibody, an antibody fragment, a VHH, or a bispecific variation of any of the foregoing.

In certain instances a human antibody is an antibody that possesses an amino acid sequence corresponding to that of an antibody produced by a human and/or has been made using any of the techniques for making human antibodies as disclosed herein. Human antibodies can be produced using various techniques known in the art, including phage-display libraries.

Hoogenboom and Winter, J. Mal. Biol., 227:381 (1991); Marks et al., J. Mal. Biol., 222 :581 (1991). Also available for the preparation of human monoclonal antibodies are methods described in Cole et al., Monoclonal Antibodies and Cancer Therapy, Alan R. Liss, p. 77 (1985); Boemer et al., J. Immunol., 147(1): 86-95 (1991). See also van Dijk and van de Winkel, Curr. Opin. Pharmacol, 2: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075.181 and 6,150,584 regarding XENOMOUSE technology). See also, for example, Li et al., Proc. Natl. Acad. Sci. USA, 103:3557-3562 (2006) regarding human antibodies generated via a human B-cell hybridoma technology.

A “humanized antibody” refers to antibodies made by a non-human cell having variable or variable and constant regions which have been altered to more closely resemble antibodies that would be made by a human cell. In some embodiments, by altering the non-human antibody amino acid sequence to incorporate amino acids found in human germline immunoglobulin sequences. The humanized antibodies of the invention can include amino acid residues not encoded by human germline immunoglobulin sequences (e.g., mutations introduced by random or site-specific mutagenesis in vitro or by somatic mutation in vivo), for example in the CDRs. Humanized antibodies can also include antibodies in which CDR sequences derived from the germline of another mammalian species, such as a mouse, have been grafted onto human framework (FR) sequences.

Humanized forms of non-human (e.g., murine) antibodies are antibodies that contain minimal sequence derived from non-human immunoglobulin. In some embodiments, a humanized antibody is a human immunoglobulin (recipient antibody) in which residues from a hypervariable of the recipient are replaced by residues from an hypervariable region of a nonhuman species (donor antibody) such as mouse, rat, rabbit, camelid, or non-human primate having the desired specificity, affinity, and/or capacity. In some instances, framework (“FR”) residues of the human immunoglobulin are replaced by corresponding non-human residues. Furthermore, humanized antibodies can comprise residues that are not found in the recipient antibody or in the donor antibody. These modifications can be made to further refine antibody performance, such as binding affinity. In general, a humanized antibody will comprise substantially all of at least one, and typically two, variable domains, in which all or substantially all of the hypervariable loops correspond to those of a non-human immunoglobulin sequence, and all or substantially all of the FR regions are those of a human immunoglobulin sequence, although the FR regions can include one or more individual FR residue substitutions that improve antibody performance, such as binding affinity, isomerization, immunogenicity, etc. The number of these amino acid substitutions in the FR are typically no more than 6 in the H chain, and in the L chain (when presente), no more than 3. The humanized antibody optionally can also include at least a portion of an immunoglobulin constant region (Fc), which can be a human immunoglobulin. Exemplary methods and humanized antibodies include those described by Jones et al. Nature 321:522-525 (1986); Riechmann et al. Nature 332:323-329 (1988); and Presta, Curr. Op. Struct. Biol. 2:593-596 (1992); Vaswani and Hamilton, Ann. Allergy. Asthma & immunol. 1: 105-115 (1998); Harris, Biochem′. Soc. Transactions 23:1035-1038 (1995); Burle and Gross, Curr. Op. Biotech.5:428-A33 (1994); and U.S. Pat. Nos. 6,982,321 and 7,087,409.

The term “functional fragment” when used in reference to an antibody refers to a portion of the antibody including heavy or light chain polypeptides that retains some or all of the binding activity as the antibody from which the fragment was derived. Such functional fragments can include, for example, an Fd, Fv, Fab, F(ab′), F(ab)₂, F(ab′)₂, single chain Fv (ScFv), diabody, triabody, tetrabody and minibody. Other functional fragments can include, for example, heavy (VHH) or light chain polypeptides, variable region polypeptides or CDR polypeptides or portions thereof so long as such functional fragments retain binding activity. Such antibody binding fragments can be found described in, for example, Harlow and Lane, Antibodies: A Laboratory Manual, Cold Spring Harbor Laboratory, New York (1989); Myers (ed.), Molec. Biology and Biotechnology: A Comprehensive Desk Reference, New York: VCH Publisher, Inc.; Huston et al., Cell Biophysics, 22:189-224 (1993); Pluckthun and Skerra, Meth. Enzymol., 178:497-515(1989) and in Day, E.D., Advanced Immunochemistry, Second Ed., Wiley-Liss, Inc., New York, NY (1990). Antibody Engineering, Second Edition, Oxford University Press, 1995.

The term “heavy chain” when used in reference to an antibody refers to a polypeptide chain of about 50-70 kDa, wherein the amino-terminal portion includes a variable region of about 120 to 130 or more amino acids and a carboxy-terminal portion that includes a constant region. The constant region can be one of five distinct types, referred to as alpha (a), delta (S), epsilon (F), gamma (7) and mu (p), based on the amino acid sequence of the heavy chain constant region. The distinct heavy chains differ in size: a, S and 7 contain approximately 450 amino acids, while p and e contain approximately 550 amino acids. When combined with a light chain, these distinct types of heavy chains give rise to five well known classes of antibodies, IgA, IgD, IgE, IgG and IgM, respectively, including four subclasses of IgG, namely IgG1, IgG2, IgG3 and IgG4. A heavy chain can be a human heavy chain or a humanized camelid heavy chain.

The term “light chain” when used in reference to an antibody refers to a polypeptide chain of about 25 kDa, wherein the amino-terminal portion includes a variable region of about 100 to about 110 or more amino acids and a carboxy-terminal portion that includes a constant region. The approximate length of a light chain is 211 to 217 amino acids. There are two distinct types, referred to as kappa (x) of lambda (k) based on the amino acid sequence of the constant domains. Light chain amino acid sequences are well known in the art. A light chain can be a human light chain.

The term “variable domain” or “variable region” refers to a portion of the light or heavy chains of an antibody that is generally located at the amino-terminal of the light or heavy chain and has a length of about 120 to 130 amino acids in the heavy chain and about 100 to 110 amino acids in the light chain, and are used in the binding and specificity of each particular antibody for its particular antigen. The variable domains can differ extensively in sequence between different antibodies. The variability in sequence is concentrated in the CDRs while the less variable portions in the variable domain are referred to as framework regions (FR). The CDRs of the light and heavy chains are primarily responsible for the interaction of the antibody with antigen. Numbering of amino acid positions used herein is according to the EU Index, as in Kabat et al. (1991) Sequences of proteins of immunological interest. (U.S. Department of Health and Human Services, Washington, D.C.) 5^{th} ed. A variable region can be a human variable region.

A CDR refers to one of three hypervariable regions (H1, H2 or H3) within the non-framework region of the immunoglobulin (Ig or antibody) VH-sheet framework, or one of three hypervariable regions (L1, L2 or L3) within the non-framework region of the antibody VL-sheet framework. Accordingly, CDRs are variable region sequences interspersed within the framework region sequences. CDR regions are well known to those skilled in the art and have been defined by, for example, Kabat as the regions of most hypervariability within the antibody variable (V) domains (Kabat et al., J. Biol. Chem. 252:6609-6616 (1977); Kabat, Adv. Prot. Chem. 32:1-75 (1978)). CDR region sequences also have been defined structurally by Chothia as those residues that are not part of the conserved-sheet framework, and thus are able to adapt different conformations (Chothia and Lesk, J. Mol. Biol. 196:901-917 (1987)). Both terminologies are well recognized in the art. The positions of CDRs within a canonical antibody variable domain have been determined by comparison of numerous structures (Al-Lazikani et al., J. Mol. Biol. 273:927-948 (1997); Morea et al., Methods 20:267-279 (2000)). Because the number of residues within a hypervariable region varies in different antibodies, additional residues relative to the canonical positions are conventionally numbered with a, b, c and so forth next to the residue number in the canonical variable domain numbering scheme (Al-Lazikani et al., supra (1997)). Such nomenclature is similarly well known to those skilled in the art.

For example, CDRs defined according to either the Kabat (hypervariable), Chothia (structural), or MacCallum (J. Mol. Biol. 262:732-745 (1996)) designations, as set forth in the Table 1 below:

TABLE 1
CDR Definitions

| | Kabat ¹ | Chothia ² | Maccallum ³ | Loop Location |
|---|---|---|---|---|
| VHCDRl | 31-35 | 26-32 | 30-35 | linking B and C strands |
| VH | 50-65 | 53-55 | 47-58 | linking C′ and C″ strands |
| CDR2 | | | | |
| VH | 95-102 | 96-101 | 93-101 | linking F and G strands |
| CDR3 | | | | |
| VLCDRl | 24-34 | 26-32 | 30-36 | linking B and C strands |
| VL CDR | 250-56 | 50-52 | 46-55 | linking C′ and C″ strands |
| VL CDR | 389-97 | 91-96 | 89-96 | linking F and G strands |

¹ Residue numbering follows the nomenclature of Kabat et al., supra.
² Residue numbering follows the nomenclature of Chothia et al., supra.
³ Residue numbering follows the nomenclature of Mccallum, which is known in the art.

The term “cancer” refers to any physiological condition in mammals characterized by unregulated cell growth. Cancers described herein include solid tumors and hematological (blood) cancers. A “hematological cancer” refers to any blood borne cancer and includes, for example, myelomas, lymphomas and leukemias. A “solid tumor” or “tumor” refers to a lesion and neoplastic cell growth and proliferation, whether malignant or benign, and all pre-cancerous and cancerous cells and tissues resulting in abnormal tissue growth. “Neoplastic,” as used herein, refers to any form of dysregulated or unregulated cell growth, whether malignant or benign, resulting in abnormal tissue growth.

The terms “treating” or “treatment” refer to any indicia of success or amelioration of the progression, severity, and/or duration of a disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; or improving a patient's physical or mental well-being.

The term “enhance” refers to an increase or improvement in the function or activity of a protein or cell after administration or contacting with a combination described herein compared to the protein or cell prior to such administration or contact.

The term “administering” refers to the act of delivering a combination or a composition described herein into a subject by such routes as oral, mucosal, topical, suppository, intravenous, parenteral, intraperitoneal, intramuscular, intralesional, intrathecal, intranasal or subcutaneous administration. Parenteral administration includes intravenous, intramuscular, intra-arterial, intradermal, subcutaneous, intraperitoneal, intraventricular, and intracranial administration.

Administration generally occurs after the onset of the disease, disorder, or condition, or its symptoms but, in certain instances, can occur before the onset of the disease, disorder, or condition, or its symptoms (e.g., administration for patients prone to such a disease, disorder, or condition).

The term “coadministration” refers to administration of two or more agents (e.g., a combination described herein and another active agent such as an anti-cancer agent described herein). The timing of coadministration depends in part of the combination and compositions administered and can include administration at the same time, just prior to, or just after the administration of one or more additional therapies, for example cancer therapies such as chemotherapy, hormonal therapy, radiotherapy, or immunotherapy. The compound of the invention can be administered alone or can be coadministered to the patient. Coadministration is meant to include simultaneous or sequential administration of the compound individually or in combination (more than one compound or agent). Thus, the preparations can also be combined, when desired, with other active substances (e.g., to reduce metabolic degradation). The compounds described herein can be used in combination with one another, with other active agents known to be useful in treating a disease associated with cells expressing a particular kinase as described herein, or with adjunctive agents that cannot be effective alone, but can contribute to the efficacy of the active agent.

The term “anti-cancer agent” is used in accordance with its plain ordinary meaning and refers to a composition having anti-neoplastic properties or the ability to inhibit the growth or proliferation of cells. In embodiments, an anti-cancer agent is a chemotherapeutic. In embodiments, an anti-cancer agent is an agent identified herein having utility in methods of treating cancer. In embodiments, an anti-cancer agent is an agent approved by the FDA or similar regulatory agency of a country other than the USA, for treating cancer.

The term “chemotherapeutic” or “chemotherapeutic agent” is used in accordance with its plain ordinary meaning and refers to a chemical composition or compound having anti neoplastic properties or the ability to inhibit the growth or proliferation of cells. “Chemotherapy” refers to a therapy or regimen that includes administration of a chemotherapeutic or anti-cancer agent described herein.

The terms “halo,” “halogen,” and “halide” refer to —F, —Cl, —Br, and —I.

The term “alkyl” by itself or as part of another substituent refers to, unless otherwise stated, a straight (i.e., unbranched) or branched carbon chain (or carbon), or combination thereof, having no unsaturation and can include mono-, di- and multivalent radicals. An alkyl as defined herein can be designated by its number of carbon atoms (i.e., C₁-C₁₀ means one to ten carbons). Alkyls herein can include C₁-C₁₀, C₁-C₆, and C₁-C₄ lengths. A “perfluoroalkyl” refers to an alkyl in which all of the hydrogens in the alkyl chain are replaced with fluoro.

The term “alkoxy” refers to an alkyl group (e.g., C₁-C₁₀, C₁-C₆, and C₁-C₄ alkyl) attached to the remainder of the molecule via an oxygen linker (—O—). Exemplary alkoxy groups include groups having the formula —OR, where R is branched or linear alkyl. A “perfluoroalkoxyl” moiety refers to an alkoxy in which all of the hydrogens in the alkyl chain are replaced with fluoro.

The term “aminoalkyl” refers to an alkyl group (e.g., C₁-C₁₀, C₁-C₆, and C₁-C₄ alkyl) in which one or more hydrogen atoms are replaced with an amino group

The term “alkylamino” refers to an alkyl group (e.g., C₁-C₁₀, C₁-C₆, and C₁-C₄ alkyl) attached to the remainder of the molecule via a nitrogen linker (—NR—). Exemplary alkylamino groups include N-methylamino, N-ethylamino, N-isopropylamino, and the like.

The term “acyl” refers to a moiety having the formula, —C(O)R, where R is a substituted or unsubstituted alkyl, haloalkyl, or amino group. The term “acylamino” refers to an acyl moiety having an attached amino group and includes, for example, such moieties as acetylamino, propionylamino, butyrylamino, isobuytrylamino, and others.

The term “alkythio” refers to an alkyl group (e.g., C₁-C₁₀, C₁-C₆, and C₁-C₄ alkyl) attached to the remainder of the molecule via a sulfur linker (—S—). Exemplary alkylthio groups include methylthio, ethylthio, propylthio, and others.

The term “heterocycle” or “heterocyclyl” refers to a stable 3- to 15-membered monocyclic group that is saturated or unsaturated and contains one or more heteroatoms (e.g., N, O, or S). Exemplary heterocycles include, but are not limited to morpholinyl, piperidinyl, piperazinyl, pyranyl, pyrrolidinyl, pyrrolinyl, imidazolidinyl, imidazolinyl, oxetanyl, azetidinyl, and others.

### Composition

Provided herein are combinations (e.g., combination therapies and compositions) useful for treating a variety of diseases, disorders, and symptoms thereof, including, for example, cancer. The combinations described herein include an MLL1-WDR5 protein-protein interaction inhibitor (WDR5 inhibitor) of Formula I, or a pharmaceutically acceptable salt thereof, and a PD-1 inhibitor such as those described below. In some embodiments, is a combination that includes a therapeutically effective amount of a PD-1 inhibitor and a therapeutically effective amount of a compound of Formula I, or a pharmaceutically acceptable salt thereof:

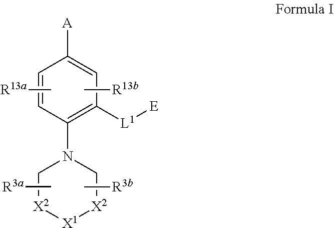

wherein:
A is

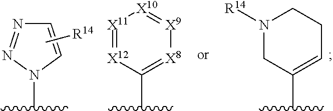

E is

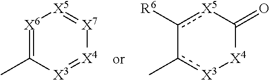

each [symbol] bond is independently a single or double bond; L¹ is —NH— or —NH—C(═O)—;
each of R^{3a}, R^{3b}, R^{13a}, R^{13b} is independently hydrogen or a substituent;
X¹ is O, NR¹, or CR^{1a}R^{1b} wherein R¹ is H, C₁-C₆ alkyl, or C₃-C₆ cycloalkyl;
X² is a bond, —CR^{2a}—, —CHR^{2b}CH₂—, or —CH₂CHR^{2c}—, wherein each of R^{2a}, R^{2b}, and R^{2e} is H, C₁-C₆ alkyl, or C₃-C₆ cycloalkyl
X³ is N or CR³, or, when the [symbol] bond attached to X³ is a single bond, X³ may also be NR³, wherein R³ is H or a substitutent;
X⁴ is N or CR⁴, wherein R⁴ is H or a substitutent;
X⁵ is N or CR⁵, or, when the [symbol] bond attached to X⁵ is a single bond, X⁵ may also be NR⁵, wherein R⁵ is H or a substitutent;
X⁶ is N or CR⁶, wherein R⁶ is H or a substitutent;
X⁷ is N or CR⁷, wherein R⁷ is H or a substitutent;
X⁸ is N or CR⁸, wherein R⁸ is H or a substitutent;
X⁹ is N or CR⁹, wherein R⁹ is H or a substitutent;
X¹⁰ is N or CR¹⁰, wherein R¹⁰ is H or a substitutent;
X¹¹ is N or CR¹¹, wherein R¹¹ is H or a substitutent;
X¹² is N or CR¹², where R¹² is H or a substitutent;
each of R^{13a} and R^{13b} is independently H or a substitutent; and
R¹⁴ is H or a substituent.

The compound of Formula I, or a pharmaceutically acceptable salt thereof, can be a compound as substantially described by U.S. patent application No's.: 63/319,564, 63/319,582, and 63/319,589 which are incorporated herein in for all purposes. In some embodiments, the compound of Formula I is 5-amino-2-chloro-4-fluoro-N-(4-fluoro-S-(4-(4-methylpiperazine-1-carbonyl)-1H-1,2,3-triazol-1-yl)-2-((3S,5R)-3,4,5-trimethylpiperazin-1-yl)phenyl)-3-methylbenzamide. In some embodiments, the combination may be formulated either as a single composition or as separate compositions for administration to a subject in need thereof (i.e., having a tumor deemed susceptible to such treatment) for the treatment of cancer.

Some embodiments described herein provide a method of treating cancer or of increasing one or more anti-tumor T cell types in a tumor in a subject having a tumor, comprising administering to the subject a combination comprising an MLL1-WDR5 protein-protein interaction inhibitor compound of Formula I, or a pharmaceutically acceptable salt thereof, and a PD-1 inhibitor. In some embodiments, the PD-1 inhibitor used in the methods described herein is an anti-PD-1 antibody. In some embodiments, the one or more T cell types comprise one or more of tumor infiltrating T lymphocytes (TILs) and cytotoxic T lymphocytes (CTLs). In some embodiments, the one or more T cell types comprise one or more CD4+ T cells, CD8+ T cells, etc. In some embodiments, a ratio of TILs to regulatory T cells (Tregs) in the tumor is increased. In some embodiments, one or more of regulatory macrophages (M2), tumor-associated myeloid cells (TAMCs), monocytic myeloid-derived suppressor cells (M-MDSCs) and polymorphonucler myeloid-derived suppressor cells (PMN-MDSCs) are reduced in the tumor. In some embodiments, the compound of Formula I has the structure:

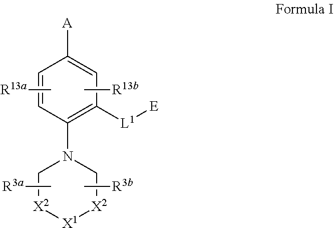

wherein:
A is

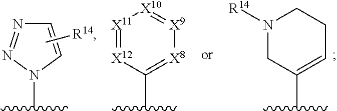

E is

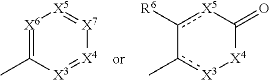

each [symbol] bond is independently a single or double bond;

### L¹ is —NH— or —NH—C(═O)—;

each of R^{3a}, R^{3b}, R^{13a}, R^{13b} is independently hydrogen or a substituent;
X¹ is O, NR¹, or CR^{1a}R^{1b} wherein R¹ is H, C₁-C₆ alkyl, or C₃-C₆ cycloalkyl;
X² is a bond, —CR^{2a}—, —CHR^{2b}CH₂—, or —CH₂CHR^{2c}—, wherein each of R^{2a}, R^{2b}, and R^{2e} is H, C₁-C₆ alkyl, or C₃-C₆ cycloalkyl;
X³ is N or CR³, or, when the [symbol] bond attached to X³ is a single bond, X³ may also be NR³, wherein R³ is H or a substitutent;
X⁴ is N or CR⁴, wherein R⁴ is H or a substitutent;
X⁵ is N or CR⁵ or, when the [symbol] bond attached to X⁵ is a single bond, X⁵ may also be NR, wherein R⁵ is H or a substitutent;
X⁶ is N or CR⁶, wherein R⁶ is H or a substitutent;
X⁷ is N or CR⁷, wherein R⁷ is H or a substitutent;
X⁸ is N or CR⁸, wherein R⁸ is H or a substitutent;
X⁹ is N or CR⁹, wherein R⁹ is H or a substitutent;
X¹⁰ is N or CR¹⁰, wherein R¹⁰ is H or a substitutent;
X¹¹ is N or CR¹¹, wherein R¹¹ is H or a substitutent;
X¹² is N or CR¹², where R¹² is H or a substitutent;
each of R^{13a} and R^{13b} is independently H or a substitutent; and
R¹⁴ is H or a substituent.

In some embodiments, the compound of Formula I, A is:

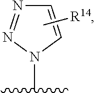

wherein R¹⁴ is a substituent with the structure:

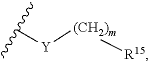

wherein Y is absent, —O—, —S—, —C(O)—, —CH₂O—, —(CO)O—, —O(CO)—, —NR¹⁵—, —C(O)NR^{15g}—or —NR^{15h}C(O)—;
m is 0 to 6;
R¹⁵ is hydrogen, amino, hydroxyl, thiol, carboxyl, cyano, C₁-C₄ alkyl, substituted C₁-C₄ alkyl, C₁-C₆ alkoxy, substituted or unsubstituted phenyl, substituted or unsubstituted C₃-C₆ cycloalkyl, substituted or unsubstituted nitrogen- or oxygen-containing 3 to 7 membered heterocyclic ring, or substituted or unsubstituted 3 to 7 membered heterocyclic ring containing both nitrogen and oxygen, wherein substituents on the heterocyclic ring can optionally be on the hetero atom; unsaturated heterocycloalkyl containing nitrogen or oxygen or both, —NR^{5a}COR^{15b}, —OR^{15c}—, —C(O)O—R^{15c}, —O(CO)O—R^{15c} —C(O)NR^{15d}R^{5e} or —NR^{15d}R^{15e}; wherein R^{15a} is hydrogen, C₁-C₄ alkyl, C₁-C₄ haloalkyl, substituted or unsubstituted phenyl, R^{15b} is hydrogen, amino, hydroxyl, C₁-C₄ alkyl, C₁-C₄ alkoxy, substituted or unsubstituted phenyl, substituted or unsubstituted nitrogen- or oxygen-containing 3 to 7 membered heterocyclic ring, wherein substituents on the heterocyclic ring can be on the hetero atom, R^{15d} and R^{15e} are each independently hydrogen, C₁-C₄ alkyl, substituted or unsubstituted phenyl, substituted or unsubstituted C₃-C₇ cycloalkyl, substituted or unsubstituted nitrogen- or oxygen-or both containing 3 to 7 membered heterocyclic ring, wherein the heterocyclic ring may be aryl, partially unsaturated, or fully saturated, wherein substituents on the heterocyclic ring can be on the hetero atom, or R^{15c} is C₁-C₄ alkyl, C₁-C₄ haloalkyl, substituted or unsubstituted phenyl, substituted or unsubstituted C₃-C₇ cycloalkyl, substituted or unsubstituted nitrogen- or oxygen- or both containing 3 to 7 membered heterocyclic ring; wherein substituents on the heterocyclic ring can be on the hetero atom; R^{15d} and R^{15c} together form: a nitrogen-, oxygen-, or nitrogen and oxygen- or nitrogen and nitrogen or oxygen and oxygen containing 3 to 7 membered heterocyclic ring, wherein the heterocyclic ring is optionally substituted with one, two or three substituents which are independently halogen, C₁-C₄ alkyl, C₁-C₄ alkoxy, amino, hydroxyl, thiol, carboxyl, cyano, trifluoromethyl or imidazolyl; wherein substituents on the heterocyclic ring can be on the hetero atom; R^{15f}, R^{15g}, and R^{15h} each independently represents hydrogen, C₁-C₄ alkyl, C₁-C₄ haloalkyl, or substituted or unsubstituted phenyl, wherein the phenyl is substituted with one, two or three of halogen, amino, cyano, hydroxyl, trifluoro C₁-C₄ alkyl, C₁-C₄ alkoxy, carboxyl, or imidazolyl.

In some embodiments, A of Formula I is:

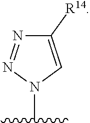

In some embodiments, Y is absent. In some embodiments, Y is —O—, —S—, —C(O)—, CH₂O—, —(CO)O—, —O(CO)—, —NR^{15f}—, —C(O)NR^{15g}—, or NR^{15h}C(O)—. In some embodiments, Y is —O—, —NR^{15f}—, or —C(O)NR^{15g}—, wherein R^{15f} and R^{15g} are independently hydrogen or C₁-C₄ alkyl.

In some embodiments, A is:

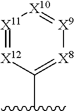

wherein:
each of R¹, R⁹, R¹⁰, R¹¹, and R¹², when present, is independly selected from H, C₁-C₆ alkyl; substituted C₁-C₆ alkyl (wherein each substituent is a 3-7-membered cycloalkyl, a 3-7-membered heterocyclic ring containing nitrogen or oxygen, or a 3-7-membered heterocyclic ring containing both nitrogen and oxygen); unsubstituted, mono-substituted, di-substituted or tri-substituted 3-7-membered heterocyclic ring containing nitrogen, oxygen or both (wherein the heterocyclic ring is aromatic, partially unsaturated or fully saturated and each substituent is independently C₁-C₆ alkyl, or NR^{D}R^{E}); —NHCOR^{E}; —CONR^{D}R^{E}; —COR^{F}; or —OR^{G}; wherein: R^{E} is: hydrogen, C₁-C₆ alkyl, 3-7-membered cycloalkyl, 3-7-membered heterocycloalkyl containing nitrogen, oxygen or both, or substituted C₁-C₆ alkyl (wherein the substituent is 3-7-membered cycloalkyl, 3-7-membered heterocyclic ring containing nitrogen or oxygen, or 3-7-membered heterocyclic ring containing both nitrogen and oxygen); R^{D}, R^{E} each independently is: hydrogen, C₁-C₆ alkyl, phenyl or substituted phenyl, substituted or unsubstituted 3-7-membered heterocyclic ring containing nitrogen, oxygen or both; substituted C₁-C₆ alkyl (wherein each substituent on R^{D} and R^{E} is independently a 3-7-membered cycloalkyl, a 3-7-membered heterocyclic ring containing nitrogen or oxygen, or a 3-7-membered heterocyclic ring containing both nitrogen and oxygen); R^{F} is: C₁-C₆ alkyl, C₁-C₆ substituted alkyl (wherein the substituent is C₃-C₆ cycloalkyl), or NR^{D}R^{E} (wherein R^{D} and R^{E} are defined in the immediate foregoing paragraph); R^{G} is: C₁-C₆ alkyl, C₁-C₆ substituted alkyl (wherein the substituent is C₃-C₆ cycloalkyl).

In some embodiments, A is:

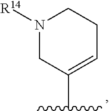

wherein R¹⁴ is a substituted 5-6-membered aromatic heterocyclic ring containing oxygen, nitrogen or both, wherein each heterocyclic ring substituent is independently C₁-C₄ alkyl, substituted C₁-C₄ alkyl (wherein the alkyl substituent is —NR^{14a}R^{14b}, wherein R^{14a} and R^{14b} are independently C₁-C₄ alkyl or 3-7-membered heterocyclic ring containing nitrogen or oxygen or both, or R^{14a} and R^{14b} are linked together to form a 3-7-membered heterocyclic ring containing nitrogen, oxygen or both).

In some embodiments, A is:

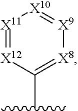

wherein:
X⁹ is N,
X¹¹ is N,
X⁸ is CR⁸, wherein R⁸ is H or a substitutent,
X¹² is CR¹², wherein R¹² is H or a substitutent,
X¹⁰ is CR¹⁰, wherein R¹⁰ is H or a substitutent.

In some embodiments, E is:

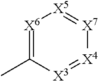

wherein each R³, R⁴, R⁵, R⁶, R⁷, when present, is independently hydrogen, halogen, amino, cyano, hydroxy, thiol, nitro, C₁-C₆ alkyl, C₃-C₇ cycloalkyl, C₁-C₆ alkoxy, C₃-C₇ cycloalkoxy, trifluoromethyl, difluoromethyl, trifluoromethoxy, difluoromethoxy, C₁-C₆ alkylthio, C₁-C₆ alkylsulfinyl, C₁-C₆ alkylsulfonyl, hydroxy-(C₁-C₆)alkyl, amino-(C₁-C₆)alkyl, or —C(O)NR^{H}R¹, wherein R^{H} and R¹ are each independently hydrogen or C₁-C₆ alkyl.

In some embodiments, E is:

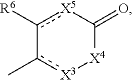

wherein each R³, R⁴, and R⁵ when present, is independently hydrogen, halogen, amino, cyano, hydroxy, thiol, nitro, C₁-C₆ alkyl, C₃-C₇ cycloalkyl, C₁-C₆ alkoxy, C₃-C₇ cycloalkoxy, trifluoromethyl, difluoromethyl, trifluoromethoxy, difluoromethoxy, C₁-C₆ alkylthio, C₁-C₆ alkylsulfinyl, C₁-C₆ alkylsulfonyl, hydroxy-(C₁-C₆)alkyl, amino-(C₁-C₆)alkyl, or —C(O)NR^{H}R¹, wherein R^{H} and R^{I} are each independently hydrogen or C₁-C₆ alkyl.

In some embodiments of the methods described herein, the compound of Formula I has a structure in which L¹ is —NH—C(═O)—. In some embodiments, L¹ is —NH—.

In some embodiments of the methods described herein, the compound of Formula I has a structure in which:
R^{3a} is hydrogen, C₁-C₆ alkyl, or C₃-C₆ cycloalkyl;
R^{3b} is hydrogen, C₁-C₆ alkyl, or C₃-C₆ cycloalkyl;
R^{13a} is hydrogen, halogen, methyl, methoxy, difluoromethoxy, or trifluoromethoxy;
R^{13b} is hydrogen, halogen, methyl, methoxy, difluoromethoxy, or trifluoromethoxy;
X¹ is NR¹, wherein R¹ is hydrogen, C₁-C₆ alkyl, or C₃-C₆ cycloalkyl;
X² is —CHR^{2a}—, —CHR^{2b}CH₂—, or —CH₂CHR^{2c}—, wherein each of R^{2a}, R^{2b}, and R^{2c} is hydrogen, C₁-C₆ alkyl, or C₃-C₆ cycloalkyl;
each of R⁴, R⁵, R⁶, and R⁷ is independently hydrogen, halogen, amino, cyano, hydroxy, thiol, nitro, C₁-C₆ alkyl, C₃-C₇ cycloalkyl, C₁-C₆ alkoxy, C₃-C₇ cycloalkoxy, trifluoromethyl, difluoromethyl, trifluoromethoxy, difluoromethoxy, C₁-C₆ alkylthio, C₁-C₆ alkylsulfinyl, C₁-C₆ alkylsulfonyl, hydroxy-(C₁-C₆)alkyl, amino-(C₁-C₆)alkyl, or —C(O)NR^{A}R^{B}, wherein R^{A} and R^{B} are each independently hydrogen or C₁-C₆ alkyl.

In some embodiments of the methods described herein, the compound of Formula I has a structure in which E is:

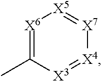

X³ is CR³, wherein R³ is H or a substitutent;
X⁴ is CR⁴, wherein R⁴ is H or a substitutent;
X⁵ is CR⁵, wherein R⁵ is H or a substitutent.

In some embodiments of the methods described herein, the compound of Formula I has a structure in which E is:

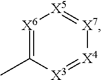

X⁶ is CR⁶, wherein R⁶ is H or a substitutent; and
X⁷ is CR⁷, wherein R⁷ is H or a substitutent.

In some embodiments of the methods described herein, the compound of Formula I has a structure in which E is:

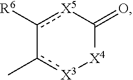

In some embodiments of the methods described herein, the compound of Formula I has a structure in which E is:

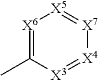

X³ is N;
X⁴ is CR⁴, wherein R⁴ is H or a substituent; and
X⁵ is CR⁵, wherein R⁵ is H or a substitutent.

In some embodiments of the methods described herein, the compound of Formula I has a structure in which E is:

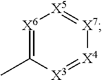

X⁶ is CR⁶, wherein R⁶ is H or a substitutent;
X⁷ is CR⁷, wherein R⁷ is H or a substitutent.

In some embodiments of the methods described herein, the compound of Formula I has a structure in which E is:

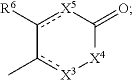

and R⁶ is H or a substituent.

In some embodiments of the methods described herein, the compound of Formula I has a structure in which:
X³ is CR³, wherein R³ is H or a substitutent;
X⁴ is N; and
X⁵ is CR⁵, wherein R⁵ is H or a substitutent.

In some embodiments of the methods described herein, the compound of Formula I has a structure in which E is:

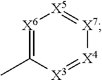

X⁶ is CR⁶, wherein R⁶ is H or a substitutent; and
X⁷ is CR⁷, wherein R⁷ is H or a substitutent.

In some embodiments of the methods described herein, the compound of Formula I has a structure in which E is:

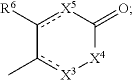

and R⁶ is H or a substituent. In some embodiments of the methods described herein, the compound of Formula I has a structure in which:
X³ is CR³, wherein R³ is H or a substitutent;
X⁴ is CR⁴, wherein R⁴ is H or a substitutent; and
X⁵ is N.

In some embodiments of the methods described herein, the compound of Formula I has a structure in which E is:

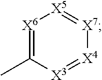

X⁶ is CR⁶, wherein R⁶ is H or a substitutent; and

X⁷ is CR⁷, wherein R⁷ is H or a substitutent.

In some embodiments of the methods described herein, the compound of Formula I has a structure in which E is:

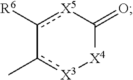

and R⁶ is H or a substituent.

In some embodiments of the methods described herein, the compound of Formula I has a structure in which:
X³ and X⁴ are N; and
X⁵ is CR⁵, wherein R⁵ is H or a substitutent.

In some embodiments of the methods described herein, the compound of Formula I has a structure in which E is:

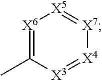

X⁶ is CR⁶, wherein R⁶ is H or a substitutent; and
X⁷ is CR⁷, wherein R⁷ is H or a substitutent.

In some embodiments of the methods described herein, the compound of Formula I has a structure in which E is:

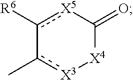

and R⁶ is H or a substituent.

In some embodiments of the methods described herein, the compound of Formula I has a structure in which E is:

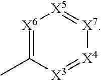

X³ is CR³, wherein R³ is H or a substitutent;
X⁴ is CR⁴, wherein R⁴ is H or a substitutent;
X⁵ is CR⁵, wherein R⁵ is H or a substitutent; and
X⁶ and X⁷ are N.

In some embodiments of the methods described herein, the compound of Formula I has a structure in which X³, X⁴, and X⁵ are N.

In some embodiments of the methods described herein, the compound of Formula I has a structure in which X⁶ is CR⁶ and X⁷ is CR⁷.

In some embodiments of the methods described herein, the compound of Formula I has a structure in which X³ is CR³, X⁴ is N, and X⁵ is CR⁵.

In some embodiments of the methods described herein, the compound of Formula I has a structure in which each of R³, R⁴, R⁵, R⁶, and R⁷ is hydrogen, halogen, amino, cyano, hydroxy, thiol, nitro, C₁-C₆ alkyl, C₃-C₇ cycloalkyl, C₁-C₆ alkoxy, C₃-C₇ cycloalkoxy, trifluoromethyl, difluoromethyl, trifluoromethoxy, difluoromethoxy, C₁-C₆ alkylthio, C₁-C₆ alkylsulfinyl, C₁-C₆ alkylsulfonyl, hydroxy-(C₁-C₆)alkyl, amino-(C₁-C₆)alkyl, or —C(O)NR^{A}R^{B}, wherein R^{A} and R^{B} are each independently hydrogen or C₁-C₆ alkyl.

In some embodiments of the methods described herein, the compound of Formula I has a structure in which each of R³, R⁵, R⁶ and R⁷ is independently hydrogen, halogen, C₁-C₆ alkyl, C₃-C₇ cycloalkyl, C₁-C₆ alkoxy, C₃-C₇ cycloalkoxy, trifluoromethyl, difluoromethyl, trifluoromethoxy, difluoromethoxy, C₁-C₆ alkylthio, C₁-C₆ alkylsulfinyl, C₁-C₆ alkylsulfonyl, nitro or cyano.

In some embodiments of the methods described herein, the compound of Formula I has a structure in which E is:

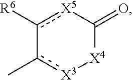

wherein each of R³, R⁴, R⁵, R⁶, and R⁷ is hydrogen, halogen, cyano, nitro, C₁-C₆ alkyl, C₃-C₇ cycloalkyl, C₁-C₆ alkoxy, C₃-C₇ cycloalkoxy, trifluoromethyl, difluoromethyl, trifluoromethoxy, difluoromethoxy, C₁-C₆ alkylthio, C₁-C₆ alkylsulfinyl, or C₁-C₆ alkylsulfonyl.

In some embodiments of the methods described herein, the compound of Formula I has a structure in which X³ is NIT.

In some embodiments of the methods and combinations described herein, the compound of Formula I is selected from Formula (Ia), Formula (Ib), Formula (Ic), or Formula (Id), or a pharmaceutically acceptable salt thereof:

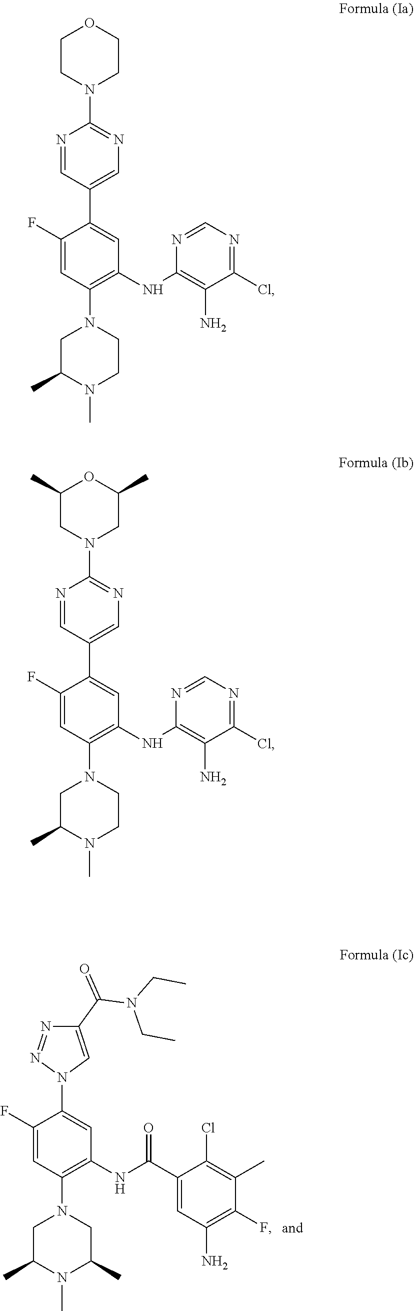 [SMILES: C[C@H]1CN(c2cc(F)c(-c3cnc(N4CCOCC4)nc3)cc2Nc2ncnc(Cl)c2N)CCN1C ; C[C@@H]1CN(c2ncc(-c3cc(Nc4ncnc(Cl)c4N)c(N4CCN(C)[C@@H](C)C4)cc3F)cn2)C[C@H](C)O1 ; CCN(CC)C(=O)c1cn(-c2cc(NC(=O)c3cc(N)c(F)c(C)c3Cl)c(N3C[C@@H](C)N(C)[C@@H](C)C3)cc2F)nn1 ; "and"]

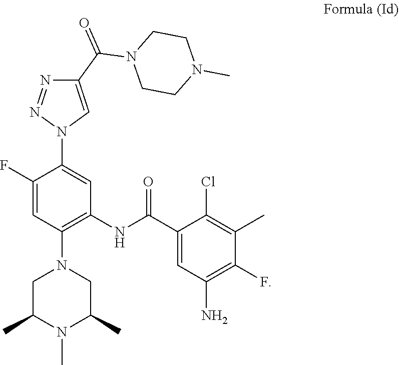 [SMILES: Cc1c(F)c(N)cc(C(=O)Nc2cc(-n3cc(C(=O)N4CCN(C)CC4)nn3)c(F)cc2N2C[C@@H](C)N(C)[C@@H](C)C2)c1Cl]

In some embodiments of the methods described herein, the compound of Formula I is a compound of Formula (Id):

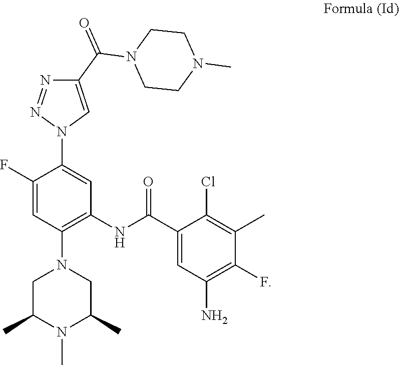 [SMILES: Cc1c(F)c(N)cc(C(=O)Nc2cc(-n3cc(C(=O)N4CCN(C)CC4)nn3)c(F)cc2N2C[C@@H](C)N(C)[C@@H](C)C2)c1Cl]

In some embodiments of the methods described herein:
a. the compound of Formula I, or a pharmaceutically acceptable salt thereof, is formulated in a first pharmaceutical composition comprising the compound of Formula I, or a pharmaceutically acceptable salt, and pharmaceutically acceptable excipient; and
b. the PD-1 inhibitor is formulated in a second pharmaceutical composition comprising the PD-1 inhibitor, or a pharmaceutically acceptable salt thereof, and pharmaceutically acceptable excipient.

In some embodiments the compound of Formula I has the Formula (Ia) as set forth below:

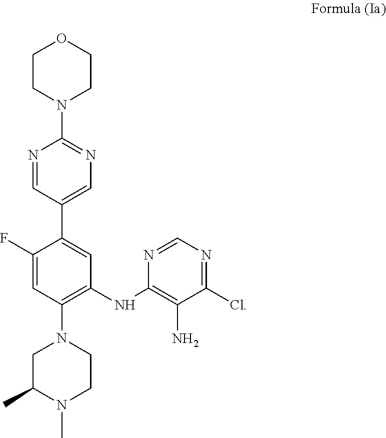 [SMILES: C[C@H]1CN(c2cc(F)c(-c3cnc(N4CCOCC4)nc3)cc2Nc2ncnc(Cl)c2N)CCN1C]

In some embodiments the compound of Formula I has the Formula (Ib) as set forth below:

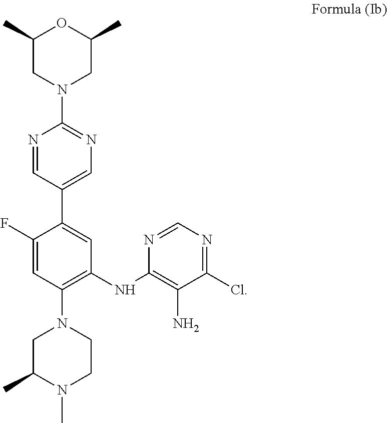 [SMILES: C[C@@H]1CN(c2ncc(-c3cc(Nc4ncnc(Cl)c4N)c(N4CCN(C)[C@@H](C)C4)cc3F)cn2)C[C@H](C)O1]

In some embodiments the compound of Formula I has the Formula (Ic) as set forth below:

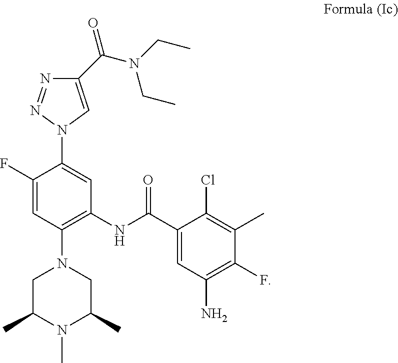 [SMILES: CCN(CC)C(=O)c1cn(-c2cc(NC(=O)c3cc(N)c(F)c(C)c3Cl)c(N3C[C@@H](C)N(C)[C@@H](C)C3)cc2F)nn1]

In some embodiments the compound of Formula I has the Formula (Id) as set forth below.

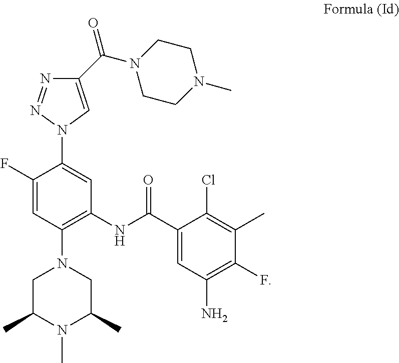 [SMILES: Cc1c(F)c(N)cc(C(=O)Nc2cc(-n3cc(C(=O)N4CCN(C)CC4)nn3)c(F)cc2N2C[C@@H](C)N(C)[C@@H](C)C2)c1Cl]

Compounds of Formula I as described herein include pharmaceutically acceptable salts, pharmaceutically acceptable stereoisomers, prodrugs, enantiomers, diastereomers, hydrates, co-crystals, and polymorphs thereof. Unless otherwise specified herein, the recitation of a compound within the scope of Formula I implies the pharmaceutically acceptable salts, pharmaceutically acceptable stereoisomers, prodrugs, enantiomers, diastereomers, hydrates, co-crystals, and polymorphs thereof. Also, where a compound within the scope of Formula I admits of a tautomeric form, recitation of a particular tautomer implies the other tautomer(s) as well.

In certain instances, the combination includes a compound of Formula I, or a pharmaceutically acceptable salt thereof, (e.g., a compound of Formula I(d), or a pharmaceutically acceptable salt thereof) present in an amount of greater than about: 1 mg, 2 mg, 3 mg, 4 mg, 5 mg, 10 mg, 15 mg, 20 mg, 25 mg, 30 mg, 35 mg, 40 mg, 45 mg, 50 mg, 60 mg, 70 mg, 80 mg, 85 mg, 90 mg, 100 mg, 125 mg, 150 mg, 175 mg, or 200 mg (based on the free base form.) The combination can include a compound of Formula I, or a pharmaceutically acceptable salt thereof, present in an amount greater than about: 1 mg, 2 mg, 3 mg, 4 mg, 5 mg, 6 mg, 7 mg, 8 mg, 9 mg, or 10 mg. In certain instances the compound of Formula I, or a pharmaceutically acceptable salt thereof, is present in an amount greater than about 5 mg or about 10 mg. The combination can include a compound of Formula I, or a pharmaceutically acceptable salt thereof, present in an amount greater than about: 1 mg to about 10 mg, 1 mg to about 25 mg, 1 mg to about 50 mg, 5 mg to about 10 mg, 5 mg to about 25 mg, 5 mg to about 50 mg, 10 mg to about 25 mg, 10 mg to about 50 mg, 50 mg to about 100 mg, or 100 mg to about 200 mg.

The combination can include a compound of Formula I, or a pharmaceutically acceptable salt thereof, present in an amount of at least about: 1 mg, 2 mg, 3 mg, 4 mg, 5 mg, 10 mg, 15 mg, 20 mg, 25 mg, 30 mg, 35 mg, 40 mg, 45 mg, 50 mg, 60 mg, 70 mg, 80 mg, 85 mg, 90 mg, 100 mg, 125 mg, 150 mg, 175 mg, or 200 mg. The combination can include a compound of Formula I, or a pharmaceutically acceptable salt thereof, present in an amount of at least about: 1 mg, 2 mg, 3 mg, 4 mg, 5 mg, 6 mg, 7 mg, 8 mg, 9 mg, or 10 mg. In certain instances the compound of Formula I is present in an amount of at least about 5 mg or about 10 mg. The combination can include a compound of Formula I, or a pharmaceutically acceptable salt thereof, present in an amount of at least about: 1 mg to about 10 mg, 1 mg to about 25 mg, 1 mg to about 50 mg, 5 mg to about 10 mg, 5 mg to about 25 mg, 5 mg to about 50 mg, 10 mg to about 25 mg, 10 mg to about 50 mg, 50 mg to about 100 mg, or 100 mg to about 200 mg.

The combination can include a compound present in an amount of about: 1 mg, 2 mg, 3 mg, 4 mg, 5 mg, 10 mg, 15 mg, 20 mg, 25 mg, 30 mg, 35 mg, 40 mg, 45 mg, 50 mg, 60 mg, 70 mg, 80 mg, 85 mg, 90 mg, 100 mg, 125 mg, 150 mg, 175 mg, or 200 mg. The combination can include a compound of Formula I, or a pharmaceutically acceptable salt thereof, present in an amount of about: 1 mg, 2 mg, 3 mg, 4 mg, 5 mg, 6 mg, 7 mg, 8 mg, 9 mg, or 10 mg. In certain instances the compound of Formula I, or a pharmaceutically acceptable salt thereof, is present in an amount of about 5 mg or about 10 mg. The combination can include a compound of Formula I, or a pharmaceutically acceptable salt thereof, present at an amount of about: 1 mg to about 10 mg, 1 mg to about 25 mg, 1 mg to about 50 mg, 5 mg to about 10 mg, 5 mg to about 25 mg, 5 mg to about 50 mg, 10 mg to about 25 mg, 10 mg to about 50 mg, 50 mg to about 100 mg, or 100 mg to about 200 mg.

A compound of Formula I, or a pharmaceutically acceptable salt thereof, can be present in the combinations described herein in amounts expressed as weight (of free base) of the compound (mg) relative to the body weight of the patient (kg). In some instances, the compound of Formula I, or a pharmaceutically acceptable salt thereof, is present in an amount equivalent to about: 0.0001 mg/kg to about 200 mg/kg, 0.001 mg/kg to about 200 mg/kg, 0.01 mg/kg to about 200 mg/kg, 0.01 mg/kg to about 150 mg/kg, 0.01 mg/kg to about 100 mg/kg, 0.01 mg/kg to about 50 mg/kg, 0.01 mg/kg to about 25 mg/kg, 0.01 mg/kg to about 10 mg/kg, or 0.01 mg/kg to about 5 mg/kg, 0.05 mg/kg to about 200 mg/kg, 0.05 mg/kg to about 150 mg/kg, 0.05 mg/kg to about 100 mg/kg, 0.05 mg/kg to about 50 mg/kg, 0.05 mg/kg to about 25 mg/kg, 0.05 mg/kg to about 10 mg/kg, or 0.05 mg/kg to about 5 mg/kg, 0.5 mg/kg to about 200 mg/kg, 0.5 mg/kg to about 150 mg/kg, 0.5 mg/kg to about 100 mg/kg, 0.5 mg/kg to about 50 mg/kg, 0.5 mg/kg to about 25 mg/kg, 0.5 mg/kg to about 10 mg/kg, or 0.5 mg/kg to about 5 mg/kg. In other instances the compound of Formula I, or a pharmaceutically acceptable salt thereof, is present in an amount equivalent to about: 1 mg/kg to about 200 mg/kg, 1 mg/kg to about 150 mg/kg, 1 mg/kg to about 100 mg/kg, 1 mg/kg to about 80 mg/kg, 40 mg/kg to about 80 mg/kg, 50 mg/kg to about 80 mg/kg, 60 mg/kg to about 80 mg/kg, 1 mg/kg to about 60 mg/kg, 1 mg/kg to about 50 mg/kg, 1 mg/kg to about 40 mg/kg, 1 mg/kg to about 25 mg/kg, 1 mg/kg to about 10 mg/kg, or 1 mg/kg to about 5 mg/kg.

PD-1 inhibitors useful in the combinations described herein include any molecule capable of inhibiting, blocking, abrogating or interfering with the activity or expression of PD-1. In particular, a PD-1 inhibitor can be a small molecule compound, a nucleic acid, a polypeptide, an antibody, a peptibody, a diabody, a minibody, a single-chain variable fragment (ScFv), or a functional fragment or variant thereof. In one instance the PD-1 inhibitor is a small molecule compound (e.g., a compound having a molecule weight of less than about 1000 Da.) In other instances, useful PD-1 inhibitors in the combinations described herein include nucleic acids and polypeptides. The PD-1 inhibitor can be a polypeptide (e.g., macrocyclic polypeptide) such as those exemplified in U.S. patent Application Publication No.: 2014/0294898, which is incorporated herein by reference in its entirety and for all purposes. In one example, the PD-1 inhibitor is an antibody, peptibody, diabody, minibody, ScFv, or a functional fragment thereof. In one example, the PD-1 inhibitor is AMP-224 (GSK).

AMP-224 is a recombinant fusion protein comprising an extracellular domain of the PD-1 ligand programmed cell death ligand 2 (PD-L2) and an Fc region of human IgG. Certain cancers can evade and suppress the immune system, in part, and without being bound by any particular theory, by interactions between PD-1 and B7-H1. AMP-224 appears to block this interaction and therefore appears to overcome immune suppression.

In some embodiments, the PD-1 inhibitor is an anti-PD-1 antibody. The anti-PD-1 antibody can be a monoclonal or polyclonal antibody. In certain embodiments, the anti-PD-1 antibody is a monoclonal antibody.

Anti-PD-1 antibodies may be of a suitable known type, including all types of antibodies and functional fragments thereof, including but not limited to those exemplified herein, such as, for example, human antibodies, mouse antibodies, chimeric antibodies, humanized antibodies, or chimeric humanized antibodies.

In some embodiments, the anti-PD-1 antibody is a human antibody. In other embodiments, the PD-1 antibody is a mouse antibody or a camelid antibody. In some embodiments, the PD-1 antibody is a chimeric antibody. In some embodiments, the PD-1 antibody is a humanized antibody. In other embodiments, the PD-1 antibody is a chimeric humanized antibody. The PD-1 antibody can be a human antibody or humanized antibody. The PD-1 antibody can be nivolumab, pembrolizumab, pidilizumab, REGN2810, PDR 001, or MEDI0680. In some embodiments, two or more PD-1 antibodies are administered in combination with a compound of Formula I, or a pharmaceutically acceptable salt thereof, as described herein.

The PD-1 antibody can be nivolumab. Nivolumab (marketed as OPDIVO) is a fully human monoclonal antibody directed against PD-1 with immunopotentiation activity. Without being bound by any particular theory, nivolumab binds to and blocks the activation of PD-1 by its cognate ligands, resulting in the activation of T-cells and cell-mediated immune responses against tumor cells or pathogens.

The PD-1 antibody can be pembrolizumab. Pembrolizumab (MK-3475, marketed as KEYTRUDA) is a humanized monoclonal IgG4 antibody directed against human cell surface receptor PD-1 with potential immuno-potentiating activity. Without being bound by any particular theory, pembrolizumab binds to PD-1, an inhibitory signaling receptor expressed on the surface of activated T cells, and blocks the binding to and activation of PD-1 by its cognate ligands. The blocking of binding and activity results in the activation of T-cell-mediated immune responses against tumor cells.

The PD-1 antibody can be pidilizumab. Pidilizumab (CT-011) is a humanized monoclonal antibody directed against human PD-1 with immunomodulating and antitumor activities. Without being bound by any particular theory, pidilizumab blocks interaction between the receptor PD-1 with its ligands, resulting in the attenuation of apoptotic processes in lymphocytes, primarily effector/memory T cells, and the augmentation of the anti-tumor activities of NK cells. The PD-I antibody can be REGN2810. REGN2810 is a human monoclonal antibody directed against PD-1, with potential immune checkpoint inhibitory and anti-neoplastic activity. Without being bound by any particular theory, REGN2810 binds to PD-1, inhibits binding to its cognate ligand, and prevents the activation of its downstream signaling pathways. This can restore immune function through the activation of cytotoxic T-cells.

The PD-I antibody can be PDR 001. PDR 001 is a fully humanized monoclonal antibody directed against PD-1, with immune checkpoint inhibitory and anti-neoplastic activities. Without being bound by any particular theory, PDR 001 binds to PD-1 expressed on activated T cells and blocks the interaction with its cognate ligands. The inhibition of ligand binding prevents PD-1-mediated signaling and results in both T-cell activation and the induction of T cell-mediated immune responses against tumor cells.

The PD-1 antibody can be MEDI0680. MEDIO680 (AMP-514) is a monoclonal antibody directed against the PD-1, with potential immunomodulating and anti-neoplastic activity. Without being bound by any particular theory, MEDIO680 appears to inhibit the activation of PD-1 and its downstream signaling pathways. This inhibition can restore immune function through the activation both of T-cells and cell-mediated immune responses against PD-1 overexpressing tumor cells.

An anti-PD-1 antibody can be of any antibody isotype. The term isotype refers to the antibody class that is encoded by heavy chain constant region genes. The heavy chains of a given antibody or functional fragment determine the class of that antibody or functional fragment: IgM, IgG, IgA, IgD or IgE. The term subclass refers to the minor differences in amino acid sequences of the heavy chains that differentiate the subclasses. In humans there are two subclasses of IgA (subclasses IgA1 and IgA2) and there are four subclasses of IgG (subclasses IgG1, IgG2, IgG3 and IgG4). Such classes and subclasses are well known to those skilled in art.

Useful anti-PD-1 antibodies (or “PD-1 antibodies”) bind to PD-1 with sufficient strength to inhibit activity of PD-1. The term “bind,” as used herein, refers to an interaction between molecules to form a complex. Interactions can be, for example, non-covalent interactions including hydrogen bonds, ionic bonds, hydrophobic interactions, and/or van der Waals interactions. A complex can also include the binding of two or more molecules held together by covalent or non-covalent bonds, interactions or forces. Binding of an antibody or functional fragment thereof can be detected using, for example, an enzyme-linked immunosorbent assay (ELISA) or any one of a number of methods that are that are well known to those skilled in the art

The strength of the total non-covalent interactions between a single antigen-binding site on an anti-PD-1 antibody or functional fragment and a single epitope of a target molecule, such as PD-1, is the affinity of the antibody or functional fragment for that epitope. The ratio of association (kl) to dissociation (k.i.) of an antibody or functional fragment thereof to a monovalent antigen (kl/k.i.) is the association constant K, which is a measure of affinity. The value of K varies for different complexes of antibody or functional fragment and antigen and depends on both ki and k.i. The association constant K for an antibody or functional fragment of the invention can be determined using any method provided herein or any other method well known to those skilled in the art.

The affinity at one binding site does not always reflect the true strength of the interaction between an antibody or functional fragment and an antigen. When complex antigens containing multiple, repeating antigenic determinants come in contact with antibodies containing multiple binding sites, the interaction of such an antibody or functional fragment with antigen at one site will increase the probability of a reaction at a second site. The strength of such multiple interactions between a multivalent antibody and antigen is called the avidity. The avidity of an antibody or functional fragment can be a better measure of its binding capacity than is the affinity of its individual binding sites. For example, high avidity can compensate for low affinity as is sometimes found for pentameric IgM antibodies, which can have a lower affinity than IgG, but the high avidity of IgM, resulting from its multivalence, enables it to bind antigen effectively.

The anti-PD-1 antibody can be administered to a patient (present) in an amount proportional to the weight of the patient in need thereof. It is customary in the art to express such proportions in terms of miligrams (mg) of drug administered per kilogram (kg) of the patient's body mass. In some embodiments, the anti-PD-1 antibody can be administered to the patient in an amount of about: 0.1 mg/kg to about 30 mg/kg, 0.1 mg/kg to about 25 mg/kg, 0.1 mg/kg to about 20 mg/kg, 0.1 mg/kg to about 15 mg/kg, 0.1 mg/kg to about 10 mg/kg, 0.1 mg/kg to about 7.5 mg/kg, 0.1 mg/kg to about 5 mg/kg, 0.1 mg/kg to about 2.5 mg/kg, or about 0.1 mg/kg to about 1 mg/kg, wherein the numerator indicates the mass of the administered drug (in mg) and the denominator indicates the patient's body mass in (kg). In some embodiments, the anti-PD-1 antibody can be administered in an amount of about: 0.5 mg/kg to about 30 mg/kg, 0.5 mg/kg to about 25 mg/kg, 0.5 mg/kg to about 20 mg/kg, 0.5 mg/kg to about 15 mg/kg, 0.5 mg/kg to about 10 mg/kg, 0.5 mg/kg to about 7.5 mg/kg, 0.5 mg/kg to about 5 mg/kg, 0.5 mg/kg to about 2.5 mg/kg, or about 0.5 mg/kg to about 1 mg/kg. In some embodiments, the anti-PD-1 antibody can be administered in an amount of about 0.5 mg/kg to about 5 mg/kg or about 0.1 mg/kg to about 10 mg/kg. In some embodiments, the anti-PD-1 antibody can be administered in an amount of about 0.5 mg/kg to about 15 mg/kg or about 0.1 mg/kg to about 20 mg/kg.

In some embodiments, the anti-PD-1 antibody can be administered to the patient at an amount of about: 0.1 mg/kg, 0.5 mg/kg, 1 mg/kg, 2 mg/kg, 3 mg/kg, 4 mg/kg, 5 mg/kg, 10 mg/kg, 15 mg/kg, 20 mg/kg or 30 mg/kg. The anti-PD-1 antibody can be administered at an amount of about: 1 mg/kg, 2 mg/kg, 3 mg/kg, 5 mg/kg or 10 mg/kg.

The dose of the anti-PD-1 antibody may also be expressed in absolute terms, e.g., as the number of miligrams (mg) of antibody per dose. In some embodiments, for example, the anti-PD-1 antibody can be administered in the combination at an amount of about: 1 mg, 5 mg, 10 mg, 15 mg, 20 mg, 25 mg, 30 mg, 40 mg, 50 mg, 60 mg, 70 mg, 75 mg, 80 mg, 90 mg, 100 mg, 150 mg, 200 mg, 250 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, or 2000 mg. The anti-PD-1 antibody can be administered in the combination in an amount of about: 1 mg to about 10 mg, 10 mg to about 20 mg, 25 mg to about 50 mg, 30 mg to about 60 mg, 40 mg to about 50 mg, 50 mg to about 100 mg, 75 mg to about 150 mg, 100 mg to about 200 mg, 200 mg to about 500 mg, 500 mg to about 1000 mg, 1000 mg to about 1200 mg, 1000 mg to about 1500 mg, 1200 mg to about 1500 mg, or 1500 mg to about 2000 mg.

The concentration of anti-PD-1 antibody in the administered combination may be expressed in mass/volume terms, e.g. as miligrams per milliliter (mg/mL). The concentration of the anti-PD-1 antibody may be varied to optimize patient tolerance. In some embodiments, the anti-PD-1 antibody can be present in the combination in an amount of about: 0.1 mg/mL, 0.5 mg/mL, 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, 15 mg/mL, 20 mg/mL, 25 mg/mL, 30 mg/mL, 40 mg/mL, 50 mg/mL, 60 mg/mL, 70 mg/mL, 80 mg/mL, 90 mg/mL, 100 mg/mL, 150 mg/mL, 200 mg/mL, 250 mg/mL, 300 mg/mL, 400 mg/mL, or 500 mg/mL. In some embodiments, the anti-PD-1 antibody is present in the combination in an amount of about: 1 mg/mL to about 10 mg/mL, 5 mg/mL to about 10 mg/mL, 5 mg/mL to about 15 mg/mL, 10 mg/mL to about 25 mg/mL; 20 mg/mL to about 30 mg/mL; 25 mg/mL to about 50 mg/ml, or 5 mg/mL to about 100 mg/mL.

The anti-PD-1 antibody may be an approved therapeutic agent, such as nivolumab or pembrolizumab. Thus, in certain instances the therapeutically effective amount of an anti-PD-1 antibody is determined as an amount provided in a package insert provided with the approved anti-PD-1 antibody. The term package insert refers to instructions customarily included in commercial packages of medicaments approved by the FDA (or a similar regulatory agency of a country or treaty region other than the USA), which contains information about, for example, the usage, dosage, administration, contraindications, and/or warnings concerning the use of such medicaments.

Compounds of Formula I as described herein can be provided in amounts that are synergistic with the amount of the PD-1 inhibitor. The term synergistic refers to a combination described herein (e.g., a compound of Formula I, or a pharmaceutically acceptable salt thereof, and a PD-1 inhibitor—including coadministration with another active agent, such as an anti-cancer agent described herein) or a combination of regimens such as those described herein that is more effective than the additive effects of each individual therapy or regimen. In evaluating effectiveness of the combination relative to its constitutent parts, one may take into account one or more therapeutic benefits as well as one or more toxicities associated with therapy.

A synergistic effect of a combination described herein can permit the use of lower dosages of one or more of the components of the combination (e.g., a compound of Formula I, or a pharmaceutically acceptable salt thereof, or a PD-1 inhibitor). A synergistic effect can permit less frequent administration of at least one of the administered therapies (e.g., a compound of Formula I, or a pharmaceutically acceptable salt thereof, or a PD-1 inhibitor) to a subject with a disease, disorder, or condition described herein. Such lower dosages and/or reduced frequency of administration can reduce the toxicity associated with the administration of at least one of the therapies (e.g., a compound of Formula I, or a pharmaceutically acceptable salt thereof, or a PD-1 inhibitor) to a subject, without reducing the efficacy of the treatment. A synergistic effect as described herein may avoid or reduce adverse or unwanted side effects associated with the use of any therapy.

A synergistic effect of a combination described herein may include efficacy against a cancer, against which one or more of the constitutents of the combination (e.g., a compound of Formula I, or a pharmaceutically acceptable salt thereof, or a PD-1 inhibitor) is (e.g., has become) ineffective by itself. In some embodiments, the synergistic effect may include enhanced efficacy and/or improved toxicity of the combination relative to one or more of its constituents in one or more types of cancer. In some embodiments, synergism may include efficacious treatment of a cancer that is resistant to one or more of the constitutents of the combination. In some embodiments, for example, the cancer may be refractory to anti-PD-1 therapy (the resistance may be inherent or acquired) alone, but respond to combined therapy with a compound of Formula I, or a pharmaceutically acceptable salt thereof, and the PD-1 inhibitor.

### Pharmaceutical Compositions

Combinations described herein can be provided as a pharmaceutical composition suitable for administration via any route to a patient described herein. Combinations described herein also include two separate pharmaceutical compositions, with each active ingredient formulated to optimize its pharmacological properties. Suitable routes of administration for such pharmaceutical compositions include, but are not limited to: oral, mucosal (e.g., nasal, inhalation, pulmonary, sublingual, vaginal, buccal, or rectal), parenteral (e.g., subcutaneous, intravenous, bolus injection, intramuscular, or intra-arterial), topical (e.g., eye drops or other ophthalmic preparations), transdermal or transcutaneous administration to a patient.

Exemplary of dosage forms include: tablets; caplets; capsules (e.g., gelatin capsules); cachets; lozenges; suppositories; powders; gels; liquid dosage forms suitable for parenteral administration to a patient; and sterile solids (e.g., crystalline or amorphous solids) that can be reconstituted to provide liquid dosage forms suitable for parenteral administration to a patient. Pharmaceutical compositions and dosage forms described herein typically include one or more excipients. Suitable excipients are well known to those skilled in the art of pharmacy. Whether a particular excipient is suitable for incorporation into a pharmaceutical composition or dosage form depends on a variety of factors such as, for example, the intended route of administration to the patient. Pharmaceutical compositions described herein can include other agents such as stabilizers, lubricants, buffers, and disintegrants that can reduce the rate by which an active ingredient can decompose in a particular formulation.

Pharmaceutical compositions described herein can in certain instances include additional active agents other than those in the combinations described herein (e.g., an anti cancer agent such as those described herein) in an amount provided herein.

In some embodiments, the compound of Formula I, or a pharmaceutically acceptable salt thereof, is provided in an oral dosage form, such as a tablet or capsule. In some embodiments, the compound of Formula I, or a pharmaceutically acceptable salt thereof, is supplied as a powder (e.g., lyophilized powder) that can be resuspended in a liquid suitable for parenteral administration.

PD-1 inhibitors described herein can be provided in forms convenient to facilitate their administration to a patient. In some embodiments, where the PD-1 inhibitor is an anti-PD-1 antibody as described herein, the PD-1 inhibitor can be formulated as a ready to use solution for parenteral administration. In some embodiments, the PD-1 inhibitor, including, for example, an anti-PD-1 antibody, can be formulated as a powder (e.g., lyophilized powder) that can be dissolved or resuspended in a liquid suitable for parenteral administration. In some embodiments, the combination includes an anti-PD-1 antibody formulated for intravenous administration. In some embodiments, the combination includes a compound of Formula I, or a pharmaceutically acceptable salt thereof, formulated as an oral dosage form (e.g., a tablet or capsule) and a PD-1 inhibitor formulated for intravenous administration.

Combinations described herein can comprise one or more controlled release pharmaceutical products, which have a goal of improving drug therapy over that achieved by their non-controlled counterparts. Controlled release formulations can extend activity of the drug, reduce dosage frequency, and increase subject compliance. In addition, controlled release formulations can be used to affect the time of onset of action or other characteristics, such as blood levels of the drug, and can thus affect the occurrence of side (e.g., adverse) effects. In some embodiments, the compound of Formula I, or a pharmaceutically acceptable salt thereof, may be provided in a controlled release formulation having an extended Tmax and/or reduced Cmax, without reduced exposure, relative to an immediate relese formulation of the same dosage (on a molar basis), in order to reduce toxicity and/or extend its period of efficacy.

### Kits

The combinations and pharmaceutical compositions described herein can be provided as part of a kit. Such kits can, for example, improve patient compliance or improve the accuracy or ease of preparation for administering the combination. The kit includes a compound of Formula I, or a pharmaceutically acceptable salt thereof, wherein the compound is supplied in a formulation as described herein. The kit also includes a PD-1 inhibitor as described herein. The kit can include AMP-224. In some embodiments, the kit includes an anti-PD-1 antibody, as described herein, such as for example, nivolumab, pembrolizumab, pidilizumab, REGN2810 (also known as SAR-439684), PDR 001, SHR-1210 or MEDI0680. Where the PD-1 inhibitor is lyophyilized, the kit can include a sterile, injectable diluent for reconstituting the lyophilized PD-1 inhibitor to form a parenteral (e.g., intravenous) dosage form. The kit can include a package insert or other information (e.g., prescribing information) useful for administration of the combination to a patient in need thereof, such as a cancer patient described herein.

Kits of the invention can include the combinations described herein (e.g., a compound of Formula I, or a pharmaceutically acceptable salt thereof, and an anti-PD-1 antibody) having the same or different formulation. Each component of a combination described herein in a kit can be supplied in a separate, individual container. Alternatively or additionally, components of the combinations described herein can be supplied in a single container. In such instances, the container can be a container that is ready for administration to a patient in need thereof, such as for example, an IV bag, ampoule, or a syringe. In some embodiments, the compound of Formula I in the kit is formulated for oral administration (e.g., a tablet, capsule, or sachet). The PD-1 inhibitor can be supplied as, for example, a powder (e.g., lyophilized powder) or as a solution for parenteral administration. In certain instances the PD-1 inhibitor is an anti-PD-1 antibody as described herein formulated for parenteral administration by, for example, intravenous administration.

One or more of the contents of kits described herein can be provided in sterile form. The kit and its contents can be provided in a form that is ready for administration to the subject in need. In such instances, the components of the combination of the kit are supplied as a formulation and optionally in an administration device such that administration requires little to no further action by the user. Where kits include administration devices, such devices include devices known and understood by those skilled in the art for routes of administration described herein, such as but not limited to, syringes, pumps, bags, cups, inhalers, droppers, patches, creams, or injectors.

### Methods

The combinations, pharmaceutical compositions, and kits described herein are useful for treating diseases, disorders, or alleviating or eliminating the symptoms of diseases and disorders such as, for example, cancer. It is to be understood that the methods described herein pertain to administration of combinations and pharmaceutical compositions described herein, and such combinations and pharmaceutical compositions can be provided in the form of a kit as described herein. Provided herein are methods of treating cancer by administering a therapeutically effective amount of a combination described herein to a patient in need thereof. Also provided herein are methods of managing cancer by administering therapeutically effective amount of a combination described herein to a patient in need thereof.

Combinations useful in the methods described herein include a compound of Formula I, or a pharmaceutically acceptable salt thereof, wherein the compound of Formula I has a structure:

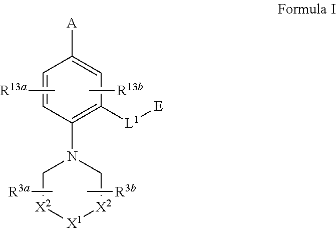

wherein:
A is

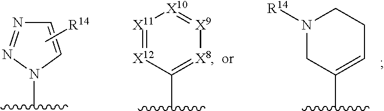

E is

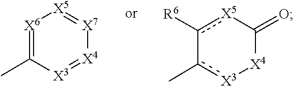

each [symbol] bond is independently a single or double bond;

### L¹ is —NH— or —NH—C(═O)—;

each of R^{3a}, R^{3b}, R^{13a}, R^{13b} is independently hydrogen or a substituent;
X¹ is O, NR¹, or CR^{1a}R^{1b} wherein R¹ is H, C₁-C₆ alkyl, or C₃-C₆ cycloalkyl;
X² is a bond, —CHR^{2a}—, —CHR^{2b}CH₂—, or —CH₂CHR^{2c}—, wherein each of R^{2a}, R^{2b}, and R^{2c} is H, C₁-C₆ alkyl, or C₃-C₆ cycloalkyl;
X³ is N or CR³, or, when the [symbol] bond attached to X³ is a single bond, X³ may also be NR³, wherein R³ is H or a substitutent;
X⁴ is N or CR⁴, wherein R⁴ is H or a substitutent;
X⁵ is N or CR⁵, or, when the [symbol] bond attached to X⁵ is a single bond, X⁵ may also be NR⁵, wherein R⁵ is H or a substitutent;
X⁶ is N or CR⁶, wherein R⁶ is H or a substitutent;
X⁷ is N or CR⁷, wherein R⁷ is H or a substitutent;
X⁸ is N or CR⁸, wherein R⁸ is H or a substitutent;
X⁹ is N or CR⁹, wherein R⁹ is H or a substitutent;
X¹⁰ is N or CR¹⁰, wherein R¹⁰ is H or a substitutent;
X¹¹ is N or CR¹¹, wherein R¹¹ is H or a substitutent;
X¹² is N or CR¹², where R¹² is H or a substitutent;
each of R^{13a} and R^{13b} is independently H or a substitutent; and
R¹⁴ is H or a substituent.

In some embodiments of Formula I, A is:

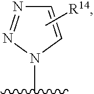

wherein R¹⁴ is a substituent with the structure:

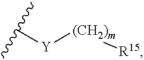

wherein Y is absent, —O—, —S—, —C(O)—, —CH₂O—, —(CO)O—, —O(CO)—, —NR^{15f}—, —C(O)NR^{15g}—, or —NR^{15h}C(O)—;
m is 0 to 6;
R¹⁵ is hydrogen, amino, hydroxyl, thiol, carboxyl, cyano, C₁-C₄ alkyl, substituted C₁-C₄ alkyl, C₁-C₆ alkoxy, substituted or unsubstituted phenyl, substituted or unsubstituted C₃-C₆ cycloalkyl, substituted or unsubstituted nitrogen- or oxygen-containing 3 to 7 membered heterocyclic ring, or substituted or unsubstituted 3 to 7 membered heterocyclic ring containing both nitrogen and oxygen, wherein substituents on the heterocyclic ring can optionally be on the hetero atom; unsaturated heterocycloalkyl containing nitrogen or oxygen or both, —NR^{15a}COR^{15b}, —OR^{15c} —, —C(O)O-R^{15c}, —O(CO)O—R^{15c} —C(O)NR^{15d}R^{15c} or —NR^{15d}R^{15c}; wherein R^{15a} is hydrogen, C₁-C₄ alkyl, C₁-C₄ haloalkyl, substituted or unsubstituted phenyl, R^{15b} is hydrogen, amino, hydroxyl, C₁-C₄ alkyl, C₁-C₄ alkoxy, substituted or unsubstituted phenyl, substituted or unsubstituted nitrogen- or oxygen-containing 3 to 7 membered heterocyclic ring, wherein substituents on the heterocyclic ring can be on the hetero atom, R^{15d} and R^{15′} are each independently hydrogen, C₁-C₄ alkyl, substituted or unsubstituted phenyl, substituted or unsubstituted C₃-C₇ cycloalkyl, substituted or unsubstituted nitrogen- or oxygen-or both containing 3 to 7 membered heterocyclic ring, wherein the heterocyclic ring may be aryl, partially unsaturated, or fully saturated, wherein substituents on the heterocyclic ring can be on the hetero atom, or R^{15c} is C₁-C₄ alkyl, C₁-C₄ haloalkyl, substituted or unsubstituted phenyl, substituted or unsubstituted C₃-C₇ cycloalkyl, substituted or unsubstituted nitrogen- or oxygen- or both containing 3 to 7 membered heterocyclic ring; wherein substituents on the heterocyclic ring can be on the hetero atom; R^{15a} and R^{15c} together form: a nitrogen-, oxygen-, or nitrogen and oxygen- or nitrogen and nitrogen or oxygen and oxygen containing 3 to 7 membered heterocyclic ring, wherein the heterocyclic ring is optionally substituted with one, two or three substituents which are independently halogen, C₁-C₄ alkyl, C₁-C₄ alkoxy, amino, hydroxyl, thiol, carboxyl, cyano, trifluoromethyl or imidazolyl; wherein substituents on the heterocyclic ring can be on the hetero atom; R^{15f}, R¹⁵ and R^{15h} each independently represents hydrogen, C₁-C₄ alkyl, C₁-C₄ haloalkyl, or substituted or unsubstituted phenyl, wherein the phenyl is substituted with one, two or three of halogen, amino, cyano, hydroxyl, trifluoro C₁-C₄ alkyl, C₁-C₄ alkoxy, carboxyl, or imidazolyl.

In some embodiments of Formula I, A is:

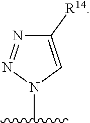

In some embodiments, Y is absent. In some embodiments, Y is —O—, —S—, —C(O)—, —CH₂O—, —(CO)O—, —O(CO)—, —NR^{15f}—, —C(O)NR^{15g}, or NR^{15h}C(O)—. In some embodiments, Y is —O—, —NR^{15f}—, or —C(O)NR^{15g}—, wherein R^{15f} and R^{15g} are independently hydrogen or C₁-C₄ alkyl.

In some embodiments of Formula I, A is:

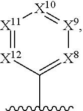

wherein:
each of R¹, R⁹, R¹⁰, R¹¹, and R¹², when present, is independly selected from H, C₁-C₆ alkyl; substituted C₁-C₆ alkyl (wherein each substituent is a 3-7-membered cycloalkyl, a 3-7-membered heterocyclic ring containing nitrogen or oxygen, or a 3-7-membered heterocyclic ring containing both nitrogen and oxygen); unsubstituted, mono-substituted, di-substituted or tri-substituted 3-7-membered heterocyclic ring containing nitrogen, oxygen or both (wherein the heterocyclic ring is aromatic, partially unsaturated or fully saturated and each substituent is independently C₁-C₆ alkyl, or NR^{D}R^{E}); —NHCOR^{E}; —CONR^{D}R^{E}; —COR^{F}; or —OR^{G}; wherein:
R^{E} is: hydrogen, C₁-C₆ alkyl, 3-7-membered cycloalkyl, 3-7-membered heterocycloalkyl containing nitrogen, oxygen or both, or substituted C₁-C₆ alkyl (wherein the substituent is 3-7-membered cycloalkyl, 3-7-membered heterocyclic ring containing nitrogen or oxygen, or 3-7-membered heterocyclic ring containing both nitrogen and oxygen);
R^{D}, R^{E} each independently is: hydrogen, C₁-C₆ alkyl, phenyl or substituted phenyl, substituted or unsubstituted 3-7-membered heterocyclic ring containing nitrogen, oxygen or both; substituted C₁-C₆ alkyl (wherein each substituent on R^{D} and R^{E} is independently a 3-7-membered cycloalkyl, a 3-7-membered heterocyclic ring containing nitrogen or oxygen, or a 3-7-membered heterocyclic ring containing both nitrogen and oxygen);
R^{F} is: C₁-C₆ alkyl, C₁-C₆ substituted alkyl (wherein the substituent is C₃-C₆ cycloalkyl), or NR^{D}R^{E} (wherein R^{D} and R^{E} are defined in the immediate foregoing paragraph); R^{G} is: C₁-C₆ alkyl, C₁-C₆ substituted alkyl (wherein the substituent is C₃-C₆ cycloalkyl).

In some embodiments of Formula I, A is:

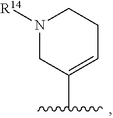

wherein R¹⁴ is a substituted 5-6-membered aromatic heterocyclic ring containing oxygen, nitrogen or both, wherein each heterocyclic ring substituent is independently C₁-C₄ alkyl, substituted C₁-C₄ alkyl (wherein the alkyl substituent is —NR^{14a}R^{14b}, wherein R^{14a} and R^{14b} are independently C₁-C₄ alkyl or 3-7-membered heterocyclic ring containing nitrogen or oxygen or both, or R^{14a} and R^{14b} are linked together to form a 3-7-membered heterocyclic ring containing nitrogen, oxygen or both).

In some embodiments, A is:
wherein:
   X¹¹ is N,
   X⁸ is CR⁸, wherein R⁸ is H or a substitutent,
   X¹² is CR¹², wherein R¹² is H or a substitutent,
   X¹⁰ is CR¹⁰, wherein R¹⁰ is H or a substitutent.

In some embodiments of Formula I, E is:

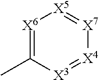

wherein each R³, R⁴, R⁵, R⁶, R⁷, when present, is independently hydrogen, halogen, amino, cyano, hydroxy, thiol, nitro, C₁-C₆ alkyl, C₃-C₇ cycloalkyl, C₁-C₆ alkoxy, C₃-C₇ cycloalkoxy, trifluoromethyl, difluoromethyl, trifluoromethoxy, difluoromethoxy, C₁-C₆ alkylthio, C₁-C₆ alkylsulfinyl, C₁-C₆ alkylsulfonyl, hydroxy-(C₁-C₆)alkyl, amino-(C₁-C₆)alkyl, or —C(O)NR^{H}R^{I}, wherein R^{H} and R^{I} are each independently hydrogen or C₁-C₆ alkyl.

In some embodiments of Formula I, E is:

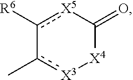

wherein each R³, R⁴, and R⁵ when present, is independently hydrogen, halogen, amino, cyano, hydroxy, thiol, nitro, C₁-C₆ alkyl, C₃-C₇ cycloalkyl, C₁-C₆ alkoxy, C₃-C₇ cycloalkoxy, trifluoromethyl, difluoromethyl, trifluoromethoxy, difluoromethoxy, C₁-C₆ alkylthio, C₁-C₆ alkylsulfinyl, C₁-C₆ alkylsulfonyl, hydroxy-(C₁-C₆)alkyl, amino-(C₁-C₆)alkyl, or —C(O)NR^{H}R^{I}, wherein R^{H} and R^{I} are each independently hydrogen or C₁-C₆ alkyl.

In some embodiments of Formula I, wherein L¹ is —NH—C(═O)—. In some embodiments of Formula I, wherein L¹ is —NH—.

In some embodiments of Formula I, wherein:
R^{3a} is hydrogen, C₁-C₆ alkyl, or C₃-C₆ cycloalkyl;
R^{3b} is hydrogen, C₁-C₆ alkyl, or C₃-C₆ cycloalkyl;
R^{13a} is hydrogen, halogen, methyl, methoxy, difluoromethoxy, or trifluoromethoxy;
R^{13b} is hydrogen, halogen, methyl, methoxy, difluoromethoxy, or trifluoromethoxy;
X¹ is NR¹, wherein R¹ is hydrogen, C₁-C₆ alkyl, or C₃-C₆ cycloalkyl;
X² is —CHR^{2a}—, —CHR^{2b}CH₂—, or —CH₂CHR^{2c}—, wherein each of R^{2a}, R^{2b}, and R^{2c} is hydrogen, C₁-C₆ alkyl, or C₃-C₆ cycloalkyl;
each of R⁴, R⁵, R⁶, and R⁷ is independently hydrogen, halogen, amino, cyano, hydroxy, thiol, nitro, C₁-C₆ alkyl, C₃-C₇ cycloalkyl, C₁-C₆ alkoxy, C₃-C₇ cycloalkoxy, trifluoromethyl, difluoromethyl, trifluoromethoxy, difluoromethoxy, C₁-C₆ alkylthio, C₁-C₆ alkylsulfinyl, C₁-C₆ alkylsulfonyl, hydroxy-(C₁-C₆)alkyl, amino-(C₁-C₆)alkyl, or —C(O)NR^{A}R^{B}, wherein R^{A} and R^{B} are each independently hydrogen or C₁-C₆ alkyl.

In some embodiments of Formula I:
X³ is CR³, wherein R³ is H or a substitutent;
X⁴ is CR⁴, wherein R⁴ is H or a substitutent;
X⁵ is CR⁵, wherein R⁵ is H or a substitutent.

In some embodiments of Formula I, E is:

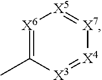

X⁶ is CR⁶, wherein R⁶ is H or a substitutent; and
X⁷ is CR⁷, wherein R⁷ is H or a substitutent.

In some embodiments of Formula I, E is:

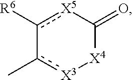

and R⁶ is H or a substituent.

In some embodiments of Formula I:

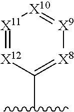

wherein:
X⁹ is N;
X¹¹ is N;
X⁸ is CR⁸, wherein R⁸ is H or a substituent; and
X¹² is CR¹², wherein R¹² is H or a substitutent.

In some embodiments of Formula I, E is:

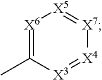

X⁶ is CR⁶, wherein R⁶ is H or a substitutent;
X⁷ is CR⁷, wherein R⁷ is H or a substitutent.

In some embodiments of Formula I, E is:

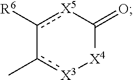

and R⁶ is H or a substituent.

In some embodiments of Formula I, wherein
X³ is CR³, wherein R³ is H or a substitutent;
X⁴ is N; and
X⁵ is CR⁵, wherein R⁵ is H or a substitutent.

In some embodiments of Formula I, E is:

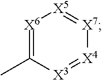

X⁶ is CR⁶, wherein R⁶ is H or a substitutent; and
X⁷ is CR⁷, wherein R⁷ is H or a substitutent.

In some embodiments of Formula I, E is:

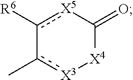

and R⁶ is H or a substituent.
In some embodiments of Formula I,
X³ is CR³, wherein R³ is H or a substitutent;
X⁴ is CR⁴, wherein R⁴ is H or a substitutent; and

### X⁵ is N.

In some embodiments of Formula I, E is:

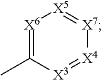

X⁶ is CR⁶, wherein R⁶ is H or a substitutent; and
X⁷ is CR⁷, wherein R⁷ is H or a substitutent.

In some embodiments of Formula I, E is:

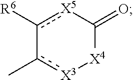

and R⁶ is H or a substituent.

In some embodiments of Formula I, wherein
X³ and X⁴ are N; and
X⁵ is CR⁵, wherein R⁵ is H or a substitutent.

In some embodiments of Formula I, E is:

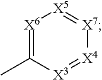

X⁶ is CR⁶, wherein R⁶ is H or a substitutent; and
X⁷ is CR⁷, wherein R⁷ is H or a substitutent.

In some embodiments of Formula I, E is:

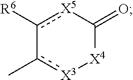

and R⁶ is H or a substituent.

In some embodiments of Formula I, E is:

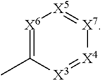

X³ is CR³, wherein R³ is H or a substitutent;
X⁴ is CR⁴, wherein R⁴ is H or a substitutent;
X⁵ is CR⁵, wherein R⁵ is H or a substitutent; and
X⁶ and X⁷ are N.

In some embodiments of Formula I, one, two, or three of X³, X⁴, and X⁵ are N. In some embodiments of Formula I, at least one of X³, X⁴, and X⁵ is N. In some embodiments of Formula I, X³, X⁴, and X⁵ is N. In some embodiments of Formula I, X⁶ is CR⁶ and X⁷ is CR⁷. In some embodiments of Formula I, X³ is CR³, X⁴ is N, and X⁵ is CR⁵. In some embodiments of Formula I, R³, R⁴, R⁵, R⁶, and R⁷ is hydrogen, halogen, amino, cyano, hydroxy, thiol, nitro, C₁-C₆ alkyl, C₃-C₇ cycloalkyl, C₁-C₆ alkoxy, C₃-C₇ cycloalkoxy, trifluoromethyl, difluoromethyl, trifluoromethoxy, difluoromethoxy, C₁-C₆ alkylthio, C₁-C₆ alkylsulfinyl, C₁-C₆ alkylsulfonyl, hydroxy-(C₁-C₆)alkyl, amino-(C₁-C₆)alkyl, or —C(O)NR^{A}R^{B}, wherein R^{A} and R^{B} are each independently hydrogen or C₁-C₆ alkyl.

In some embodiments of Formula I, each of R³, R⁵, R⁶ and R⁷ is independently hydrogen, halogen, C₁-C₆ alkyl, C₃-C₇ cycloalkyl, C₁-C₆ alkoxy, C₃-C₇ cycloalkoxy, trifluoromethyl, difluoromethyl, trifluoromethoxy, difluoromethoxy, C₁-C₆ alkylthio, C₁-C₆ alkylsulfinyl, C₁-C₆ alkylsulfonyl, nitro or cyano.

In some embodiments of Formula I, E is:

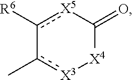

wherein
each of R³, R⁴, R⁵, R⁶, and R⁷ is hydrogen, halogen, cyano, nitro, C₁-C₆ alkyl, C₃-C₇ cycloalkyl, C₁-C₆ alkoxy, C₃-C₇ cycloalkoxy, trifluoromethyl, difluoromethyl, trifluoromethoxy, difluoromethoxy, C₁-C₆ alkylthio, C₁-C₆ alkylsulfinyl, or C₁-C₆ alkylsulfonyl.

In some embodiments of the compound of Formula I, X³ is NH.

In some embodiments, the compound of Formula I is selected from:

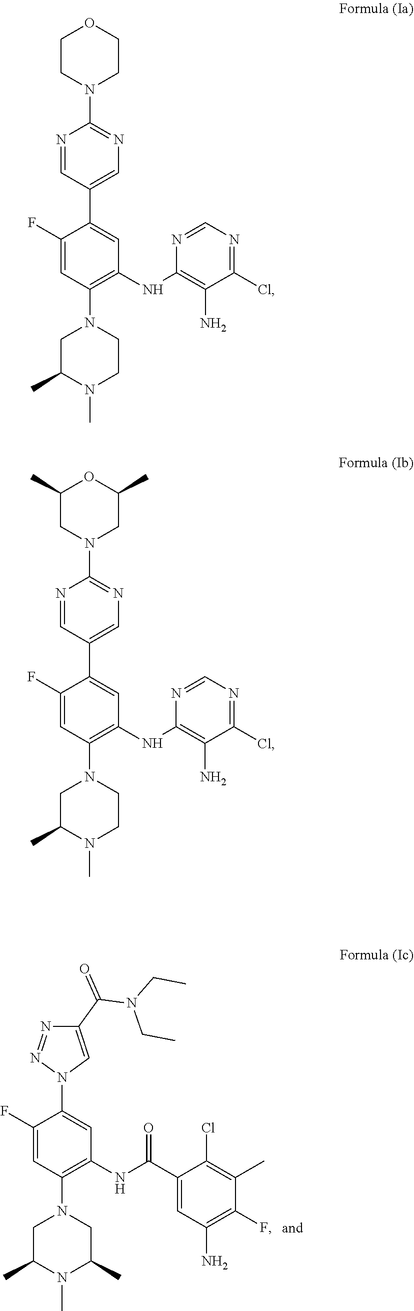 [SMILES: C[C@H]1CN(c2cc(F)c(-c3cnc(N4CCOCC4)nc3)cc2Nc2ncnc(Cl)c2N)CCN1C ; C[C@@H]1CN(c2ncc(-c3cc(Nc4ncnc(Cl)c4N)c(N4CCN(C)[C@@H](C)C4)cc3F)cn2)C[C@H](C)O1 ; CCN(CC)C(=O)c1cn(-c2cc(NC(=O)c3cc(N)c(F)c(C)c3Cl)c(N3C[C@@H](C)N(C)[C@@H](C)C3)cc2F)nn1 ; "and"]

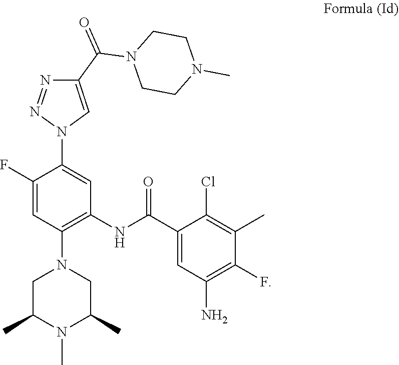 [SMILES: Cc1c(F)c(N)cc(C(=O)Nc2cc(-n3cc(C(=O)N4CCN(C)CC4)nn3)c(F)cc2N2C[C@@H](C)N(C)[C@@H](C)C2)c1Cl]

In some embodiments, the compound of Formula I is:

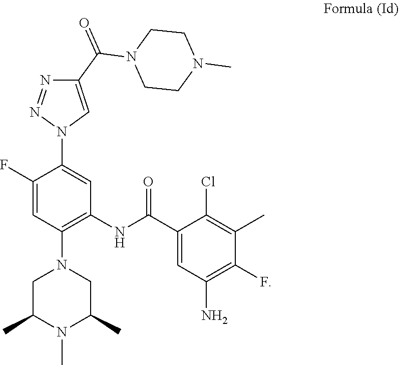 [SMILES: Cc1c(F)c(N)cc(C(=O)Nc2cc(-n3cc(C(=O)N4CCN(C)CC4)nn3)c(F)cc2N2C[C@@H](C)N(C)[C@@H](C)C2)c1Cl]

In some embodiments:
a. the compound of Formula I is formulated in a first pharmaceutical composition comprising the compound of Formula I, or a pharmaceutically acceptable salt, and pharmaceutically acceptable excipient; and
b. the PD-1 inhibitor is formulated in a second pharmaceutical composition comprising the PD-1 inhibitor, or a pharmaceutically acceptable salt thereof, and pharmaceutically acceptable excipient.

The PD-1 inhibitors for use in the methods described herein are those PD-1 inhibitors described herein. In some embodiments, the PD-1 inhibitor can be a small molecule compound, a nucleic acid, a polypeptide, an antibody, a peptibody, a diabody, a minibody, a single-chain variable fragment (ScFv), or functional fragment or variant thereof. In one example, the PD-1 inhibitor is AMP-224. In other examples, the PD-1 inhibitor can be an anti-PD-1 antibody as set forth above. In one instance, the anti-PD-1 antibody for use in the methods described herein is nivolumab, pembrolizumab, pidilizumab, REGN2810 (also known as SAR-439684), PDR 001, SHR-1210 or MEDI0680.

It should be understood that the compound of Formula I and the anti-PD-1 inhibitor constituting the combination for use in such methods includes each therapy in amounts as described herein and are administered as described herein. In some embodiments, the compound of Formula I can be present in a combination administered to patient in need thereof at an amount of about 5 mg to about 50 mg or about 5 mg to about 100 mg. In some embodiments, the PD-1 inhibitor can be an anti-PD-1 antibody present in an amount of about 0.1 mg/kg to about 10 mg/kg or about 0.1 mg/kg to about 20 mg/kg. These amounts are merely exemplary and do not limit in any way the amount of each therapy that can be present in the combination as described herein.

It is also understood that the combination for use in the methods described herein can be provided as a kit as set forth above. Such kits include each component of the combination as described herein and optionally additional kit components including, for example, containers and administration devices such as those described above.

In some embodiments, the disease or condition being treated is a cancer. In some embodiments, the cancer is a blood cancer.

### Leukemia

Leukemia is characterized by an abnormal increase of white blood cells in the blood or bone marrow. Among all types of cancers, the morbidity of leukemia is the highest for patients below 35 years old. Over 70% of infant leukemia patients bear a translocation involving chromosome 11, resulting in the fusion of the MLL1 gene with other genes (Nat. Rev. Cancer., 2007, 7(1 1):823-833). MLL1 translocations are also found in approximately 10% of adult acute myeloid leukemia (AML) patients who were previously treated with topoisomerase II inhibitors for other types of cancers.

MLL1 enzymatic activity is determined by MLL1 and WDR5 protein-protein interaction; MLL1 enzymatic activity affects the methylation level of H3K4 (histone 3 lysine 4). The H3K4 methylation level increases abnormally in MLL fusion type leukemia, and the downstream Hox and Meis-1 gene expression levels are up-regulated abnormally. When MLL1-WDR5 protein-protein interaction is inhibited, MLL1 catalytic activity decreases, H3K4 methylation level decreases, and Hox and Meis-1 gene expression levels are downregulated, inhibiting leukemia cell proliferation.

In some embodiments, the cancer is leukemia. In some embodiments, the leukemia is acute leukemia. In some embodiments, the acute leukemia is acute leukemia with MLL1 gene rearrangement.

### Acute Myeloid Leukemia (AML)

The CEBPA gene is mutated in 9% of patients with acute myeloid leukemia (AML). Selective expression of a short (30-kDa) CCAAT-enhancer binding protein-a (C/EBPa) translational isoform, termed p30, represents the most common type of CEBPA mutation in AML. The molecular mechanisms underlying p30-mediated transformation remain incompletely understood. Studies have shown that C/EBPa p30, but not the normal p42 isoform, preferentially interacts with WDR5, a key component of SET/MLL (SET-domain/mixed-lineage leukemia) histone-methyltransferase complexes. Accordingly, p30-bound genomic regions are enriched for MLL-dependent H3K4me3 marks. The p30-dependent increase in self-renewal and inhibition of myeloid differentiation required WDR5, as downregulation of the latter inhibited proliferation and restored differentiation in p30-dependent AML models. Small-molecule inhibitors of WDR5-MLL binding selectively inhibited proliferation and induced differentiation in p30-expressing human AML cells revealing the mechanism of p30-dependent transformation and establish the p30 cofactor WDR5 as a therapeutic target in CEBPA-mutant AML (Nat. Chem. Biol. 2015; 11(8): 571-8). Thus, in some embodiments, the leukemia treated with the combination of WDR5 inhibitor of Formula I and PD-1 inhibitor is AML leukemia.

### MYCN-amplified Neuroblastoma

MYCN gene amplification in neuroblastoma drives a gene expression program that correlates strongly with aggressive disease. Mechanistically, trimethylation of histone H3 lysine 4 (H3K4) at target gene promoters is a prerequisite for the transcriptional program to be enacted. WDR5 is a histone H3K4 presenter that has been found to have an essential role in H3K4 trimethylation. The relationship between WDR5-mediated H3K4 trimethylation and N-Myc transcriptional programs in neuroblastoma cells was investigated. N-Myc upregulated WDR5 expression in neuroblastoma cells. Gene expression analysis revealed that WDR5 target genes included those with MYC-binding elements at promoters such as MDM2. WDR5 has been shown to form a protein complex at the MDM2 promoter with N-Myc, but not p53, leading to histone H3K4 trimethylation and activation of MDM2 transcription (Cancer Res. 2015; 75(23); 5143-54). RNAi-mediated attenuation of WDR5 upregulated expression of wild-type but not mutant p53, an effect associated with growth inhibition and apoptosis. Similarly, a small-molecule antagonist of WDR5 reduced N-Myc/WDR5 complex formation, N-Myc target gene expression, and cell growth in neuroblastoma cells. In MYCN-transgenic mice, WDR5 was overexpressed in precancerous ganglion and neuroblastoma cells compared with normal ganglion cells. Clinically, elevated levels of WDR5 in neuroblastoma specimens have been an independent predictor of poor overall survival. WDR5 has been identified as a relevant cofactor for N-Myc-regulated transcriptional activation and tumorogenesis and as a novel therapeutic target for MYCN-amplified neuroblastomas (Cancer Res 2015; 75(23); 5143-54, Mol. Cell. 2015; 58(3): 440-52). Thus, in some embodiments, the cancer treated with a combination of a WDR5 inhibitor of Formula I and a PD-1 inhibitor is a solid tumor. In some embodiments, the treated cancer is a neuroblastoma.

In some embodiments, cancer can be a solid tumor. The cancer can be a hematological cancer, in some embodiments. In some instances, the cancer treated with a combination of a WDR5 inhibitor of Formula I and a PD-1 inhibitor is a solid tumor selected from the group consisting of squamous cell carcinoma, nonsquamous cell carcinoma, non-small cell lung cancer (NSCLC), small cell lung cancer, melanoma, hepatocellular carcinoma, renal cell carcinoma, ovarian cancer, head and neck cancer, urothelial cancer, breast cancer, prostate cancer, glioblastoma, colorectal cancer, pancreatic cancer, lymphoma, leiomyosarcoma, liposarcoma, synovial sarcoma, or malignant peripheral sheath tumor (MPNST).

In particular embodiments, the cancer treated with a combination of a WDR5 inhibitor of Formula I and a PD-1 inhibitor is a solid tumor selected from non-small cell lung cancer (NSCLC), hepatocellular carcinoma, melanoma, ovarian cancer, breast cancer, pancreatic cancer, renal cell carcinoma, or colorectal cancer. The treated cancer can be non-small cell lung cancer (NSCLC). The treated cancer can be hepatocellular carcinoma. The treated cancer can be melanoma. The cancer can be ovarian cancer. The treated cancer can be breast cancer. The treated cancer can be pancreatic cancer. The treated cancer can be renal cell carcinoma. The treated cancer can be colorectal cancer.

Provided herein are methods of treating NSCLC by administering a therapeutically effective amount of a combination described herein, wherein the combination includes a compound of Formula I and an anti-PD-1 antibody selected from nivolumab, pembrolizumab, pidilizumab, REGN2810 (also known as SAR-439684), PDR 001, SHR-1210 or MEDI0680. In some embodiments, the method includes treating NSCLC by administering AMP-224 in combination with a compound of Formula I described herein. In some embodiments, the method includes treating NSCLC by administering nivolumab or pembrolizumab in combination (e.g., in separate pharmaceutical compositions) with a compound of Formula I described herein. In some embodiments, the NSCLC is Stage IIA or Stage IIB. The NSCLC can be a Stage IIIA or Stage IIIB cancer. The NSCLC can be a Stage IV cancer. Staging of cancers as described herein is described by the American Joint Committee on Cancer TNM classification of malignant tumors cancer staging notation as is well understood in the art. Those of skill in the art will readily understand other staging classification systems are available and applicable to the methods described herein. In certain instances, the method is a method of treating Stage IIIA or IIIB NSCLC by administering a combination described herein that includes a compound of Formula I and an anti-PD-1 antibody selected from nivolumab, pembrolizumab, pidilizumab, REGN2810 (also known as SAR-439684), PDR 001, SHR-1210 or MEDI0680. In some embodiments, the method includes treating Stage IIIA or IIIB NSCLC by administering AMP-224 in combination with a compound of Formula I described herein. Some embodiments include a method of treating a Stage IV NSCLC by administering a combination described herein that includes a compound of Formula I and an anti-PD-1 antibody selected from nivolumab, pembrolizumab, pidilizumab, REGN2810 (also known as SAR-439684), PDR 001, SHR-1210 or MEDI0680. In some embodiments, the method includes treating Stage IV NSCLC by administering AMP-224 in combination with a compound of Formula I described herein. In some embodiments, the method includes treating Stage IV NSCLC by administering nivolumab or pembrolizumab in combination (e.g., in a separate pharmaceutical composition) with a compound of Formula I described herein.

Further provided herein are methods of treating hepatocellular carcinoma by administering a therapeutically effective amount of a combination described herein, wherein the combination includes a compound of Formula I and an anti-PD-1 antibody selected from nivolumab, pembrolizumab, pidilizumab, REGN2810 (also known as SAR-439684), PDR 001, SHR-1210 or MEDI0680. In some embodiments, the method includes treating hepatocellular carcinoma by administering AMP-224 in combination with a compound of Formula I described herein. In some embodiments, the treated hepatocellular carcinoma is a Stage II cancer. In some embodiments, the hepatocellular carcinoma is a Stage IIIA, Stage IIIB, or Stage IIIC cancer. In some embodiments, the hepatocellular carcinoma is a Stage IVA or Stage IVB cancer. In some embodiments, the method is a method of treating Stage III (e.g., Stage IIIA, IIIB, or IIIC) hepatocellular carcinoma by administering a therapeutically effective amount of a combination described herein where the combination includes a compound of Formula I and an anti-PD-1 antibody selected from nivolumab, pembrolizumab, pidilizumab, REGN2810 (also known as SAR-439684), PDR 001, SHR-1210 or MEDI0680. In some embodiments, the method includes treating Stage III (e.g., Stage IIIA, IIIB, or IIIC) hepatocellular carcinoma by administering AMP-224 in combination with a compound of Formula I described herein. Some embodiments comprise a method of treating Stage IV (e.g., Stage IVA or Stage IVB) hepatocellular carcinoma by administering a therapeutically effective amount of a combination described herein where the combination includes a compound of Formula I and an anti-PD-1 antibody selected from nivolumab, pembrolizumab, pidilizumab, REGN2810 (also known as SAR-439684), PDR 001, SHR-1210 or MEDI0680. In some embodiments, the method includes treating Stage IV (e.g., Stage IVA or Stage IVB) hepatocellular carcinoma by administering AMP-224 in combination with a compound of Formula I described herein.

Further provided herein are methods of treating melanoma by administering a therapeutically effective amount of a combination described herein where the combination includes a compound of Formula I and an anti-PD-1 antibody selected from nivolumab, pembrolizumab, pidilizumab, REGN2810 (also known as SAR-439684), PDR 001, SHR-1210 or MEDI0680. In some embodiments, the method includes treating melanoma by administering AMP-224 in combination with a compound of Formula I described herein. In some embodiments, the melanoma is a Stage IIA, IIB, or IIC cancer. In some embodiments, the melanoma is a Stage IIIA, Stage IIIB, or Stage IIIC cancer. In some embodiments, the melanoma is a Stage IV cancer. In some embodiments, the method is a method of treating Stage II (e.g., Stage IIA, IIB, or IIC) melanoma by administering a therapeutically effective amount of a combination described herein where the combination includes a compound of Formula I and an anti-PD-1 antibody selected from nivolumab, pembrolizumab, pidilizumab, REGN2810 (also known as SAR-439684), PDR 001, SHR-1210 or MEDI0680. In some embodiments, the method includes treating Stage II (e.g., Stage IIA, IIB, or IIC) melanoma by administering AMP-224 in combination with a compound of Formula I described herein. In some embodiments, the method is a method of treating Stage III (e.g., Stage IIIA, IIIB, or IIIC) melanoma by administering a therapeutically effective amount of a combination described herein where the combination includes a compound of Formula I and an anti-PD-1 antibody selected from nivolumab, pembrolizumab, pidilizumab, REGN2810 (also known as SAR-439684), PDR 001, SHR-1210 or MEDI0680. In some embodiments, the method includes treating Stage III (e.g., Stage IIIA, IIIB, or IIIC) melanoma by administering AMP-224 in combination with a compound of Formula I described herein. Some embodiments provide a method of treating Stage IV melanoma by administering a therapeutically effective amount of a combination described herein where the combination includes a compound of Formula I and an anti-PD-1 antibody selected from nivolumab, pembrolizumab, pidilizumab, REGN2810 (also known as SAR-439684), PDR 001, SHR-1210 or MEDI0680. In some embodiments, the method includes treating Stage IV melanoma by administering AMP-224 in combination with a compound of Formula I described herein.

Some embodiments include methods of treating ovarian cancer by administering a therapeutically effective amount of a combination described herein where the combination includes a compound of Formula I, or a pharmaceutically acceptable salt thereof, and an anti-PD-1 antibody selected from nivolumab, pembrolizumab, pidilizumab, REGN2810 (also known as SAR-439684), PDR 001, SHR-1210 or MEDI0680. In some embodiments, the method includes treating ovarian cancer by administering AMP-224 in combination with a compound of Formula I, or a pharmaceutically acceptable salt thereof, described herein. In some embodiments, the ovarian cancer is a Stage I cancer as defined by the FIGO Ovarian Cancer Staging standards. The ovarian cancer can be a Stage IA, IB, or IC (e.g., IC1, IC2, or IC3) cancer. In some embodiments, the ovarian cancer is a Stage II cancer. The ovarian cancer can be a Stage IIA or IIB cancer. In some embodiments, the method is a method of treating Stage I (e.g., Stage IA, IB, IC1, IC2, or IC3) ovarian cancer by administering a therapeutically effective amount of a combination described herein where the combination includes a compound of Formula I, or a pharmaceutically acceptable salt thereof, and an anti-PD-1 antibody selected from nivolumab, pembrolizumab, pidilizumab, REGN2810 (also known as SAR-439684), PDR 001, SHR-1210 or MEDI0680. In some embodiments, the method includes treating Stage I (e.g., Stage IA, IB, IC1, IC2, or IC3) ovarian cancer by administering AMP-224 in combination with a compound of Formula I, or a pharmaceutically acceptable salt thereof, described herein. In some embodiments, the method is a method of treating Stage II (e.g., Stage 11A or IIB) ovarian cancer by administering a therapeutically effective amount of a combination described herein where the combination includes a compound of Formula I, or a pharmaceutically acceptable salt thereof, and an anti-PD-1 antibody selected from nivolumab, pembrolizumab, pidilizumab, REGN2810 (also known as SAR-439684), PDR 001, SHR-1210 or MEDI0680. In some embodiments, the method includes treating Stage II (e.g., Stage IIA or IIB) ovarian cancer by administering AMP-224 in combination with a compound of Formula I, or a pharmaceutically acceptable salt thereof, described herein.

Also provided herein are methods of treating breast cancer by administering a therapeutically effective amount of a combination described herein where the combination includes a compound of Formula I, or a pharmaceutically acceptable salt thereof, and an anti-PD-1 antibody selected from nivolumab, pembrolizumab, pidilizumab, REGN2810 (also known as SAR-439684), PDR 001, SHR-1210 or MEDI0680. In some embodiments, the method includes treating breast cancer by administering AMP-224 in combination with a compound of Formula I, or a pharmaceutically acceptable salt thereof, described herein. The breast cancer can be HER2 negative breast cancer. The breast cancer can be a HER2 positive breast cancer. The breast cancer can be triple-negative breast cancer. In some embodiments, the breast cancer is a Stage IA or Stage IB cancer. In some embodiments, the breast cancer is a Stage IIA or Stage IIB cancer. In some embodiments, the breast cancer is a Stage IIIA, Stage IIIB, or Stage IIIC cancer. In some embodiments, the breast cancer is a Stage IV cancer. In some embodiments, the method is a method of treating Stage I (e.g., Stage IA or IB) breast cancer by administering a therapeutically effective amount of a combination described herein where the combination includes a compound of Formula I, or a pharmaceutically acceptable salt thereof, and an anti-PD-1 antibody selected from nivolumab, pembrolizumab, pidilizumab, REGN2810 (also known as SAR-439684), PDR 001, SHR-1210 or MEDI0680. In some embodiments, the method includes treating Stage I (e.g., Stage IA or IB) breast cancer by administering AMP-224 in combination with a compound of Formula I, or a pharmaceutically acceptable salt thereof, described herein. In some embodiments, the method is a method of treating Stage II (e.g., Stage IIA or IIB) breast cancer by administering a therapeutically effective amount of a combination described herein where the combination includes a compound of Formula I, or a pharmaceutically acceptable salt thereof, and an anti-PD-1 antibody selected from nivolumab, pembrolizumab, pidilizumab, REGN2810 (also known as SAR-439684), PDR 001, SHR-1210 or MEDI0680. In some embodiments, the method includes treating Stage II (e.g., Stage IIA or IIB) breast cancer by administering AMP-224 in combination with a compound of Formula I, or a pharmaceutically acceptable salt thereof, described herein. In some embodiments, the method is a method of treating Stage III (e.g., Stage IIIA, IIIB, or IIIC) breast cancer by administering a therapeutically effective amount of a combination described herein where the combination includes a compound of Formula I, or a pharmaceutically acceptable salt thereof, and an anti-PD-1 antibody selected from nivolumab, pembrolizumab, pidilizumab, REGN2810 (also known as SAR-439684), PDR 001, SHR-1210 or MEDI0680.

In some embodiments, the method includes treating Stage I (e.g., Stage IA or IB) breast cancer by administering AMP-224 in combination with a compound of Formula I, or a pharmaceutically acceptable salt thereof, described herein. In some embodiments, the method is a method of treating Stage II (e.g., Stage IIA or IIB) breast cancer by administering a therapeutically effective amount of a combination described herein where the combination includes a compound of Formula I, or a pharmaceutically acceptable salt thereof, and an anti-PD-1 antibody selected from nivolumab, pembrolizumab, pidilizumab, REGN2810 (also known as SAR-439684), PDR 001, SHR-1210 or MEDI0680. In some embodiments, the method includes treating Stage II (e.g., Stage IIA or IIB) breast cancer by administering AMP-224 in combination with a compound of Formula I, or a pharmaceutically acceptable salt thereof, described herein. In some embodiments, the method is a method of treating Stage III (e.g., Stage IIIA, IIIB, or IIIC) breast cancer by administering a therapeutically effective amount of a combination described herein where the combination includes a compound of Formula I, or a pharmaceutically acceptable salt thereof, and an anti-PD-1 antibody selected from nivolumab, pembrolizumab, pidilizumab, REGN2810 (also known as SAR-439684), PDR 001, SHR-1210 or MEDI0680. In some embodiments, the method includes treating Stage III (e.g., Stage IIIA, IIIB, or IIIC) breast cancer by administering AMP224 in combination with a compound of Formula I, or a pharmaceutically acceptable salt thereof, described herein. In some embodiments, the method is a method of treating Stage IV breast cancer by administering a therapeutically effective amount of a combination described herein where the combination includes a compound of Formula I, or a pharmaceutically acceptable salt thereof, and an anti-PD-1 antibody selected from nivolumab, pembrolizumab, pidilizumab, REGN2810 (also known as SAR-439684), PDR 001, SHR-1210 or MEDI0680. In some embodiments, the method includes treating Stage IV breast cancer by administering AMP-224 in combination with a compound of Formula I, or a pharmaceutically acceptable salt thereof, described herein.

Methods of treating pancreatic cancer are provided herein. In some embodiments, the method includes treating pancreatic cancer by administering a therapeutically effective amount of a combination described herein where the combination includes a compound of Formula I, or a pharmaceutically acceptable salt thereof, and an anti-PD-1 antibody selected from nivolumab, pembrolizumab, pidilizumab, REGN2810 (also known as SAR-439684), PDR 001, SHR-1210 or MEDI0680. In some embodiments, the method includes treating pancreatic cancer by administering AMP-224 in combination with a compound of Formula I, or a pharmaceutically acceptable salt thereof, described herein. In some embodiments, the pancreatic cancer is locally advanced, surgically resected or unresected pancreatic cancer or metastatic pancreatic adenocarcinoma. In some embodiments, the pancreatic cancer is a Stage IA or Stage 1B cancer. In some embodiments, the pancreatic cancer is a Stage IIA or Stage IIB cancer. In some embodiments, the pancreatic cancer is a Stage III cancer. In some embodiments, the pancreatic cancer is a Stage IV cancer. In some embodiments, the method is a method of treating Stage I (e.g., Stage IA or IB) pancreatic cancer by administering a therapeutically effective amount of a combination described herein where the combination includes a compound of Formula I, or a pharmaceutically acceptable salt thereof, and an anti-PD-1 antibody selected from nivolumab, pembrolizumab, pidilizumab, REGN2810 (also known as SAR-439684), PDR 001, SHR-1210 or MEDI0680. In some embodiments, the method includes treating Stage I (e.g., Stage IA or IB) pancreatic cancer by administering AMP-224 in combination with a compound of Formula I, or a pharmaceutically acceptable salt thereof, described herein. In some embodiments, the method is a method of treating Stage II (e.g., Stage IIA or IIB) pancreatic cancer by administering a therapeutically effective amount of a combination described herein where the combination includes a compound of Formula I, or a pharmaceutically acceptable salt thereof, and an anti-PD-1 antibody selected from nivolumab, pembrolizumab, pidilizumab, REGN2810 (also known as SAR-439684), PDR 001, SHR-1210 or MEDI0680. In some embodiments, the method includes treating Stage II (e.g., Stage IIA or IIB) pancreatic cancer by administering AMP-224 in combination with a compound of Formula I, or a pharmaceutically acceptable salt thereof, described herein. In some embodiments, the method is a method of treating Stage III pancreatic cancer by administering a therapeutically effective amount of a combination described herein where the combination includes a compound of Formula I, or a pharmaceutically acceptable salt thereof, and an anti-PD-1 antibody selected from nivolumab, pembrolizumab, pidilizumab, REGN2810 (also known as SAR-439684), PDR 001, SHR-1210 or MEDI0680. In some embodiments, the method includes treating Stage III pancreatic cancer by administering AMP-224 in combination with a compound of Formula I, or a pharmaceutically acceptable salt thereof, described herein. Some embodiments include a method of treating Stage IV pancreatic cancer by administering a therapeutically effective amount of a combination described herein where the combination includes a compound of Formula I, or a pharmaceutically acceptable salt thereof, and an anti-PD-1 antibody selected from nivolumab, pembrolizumab, pidilizumab, REGN2810 (also known as SAR-439684), PDR 001, SHR-1210 or MEDI0680. In another aspect the method includes treating Stage IV pancreatic cancer by administering AMP-224 in combination with a compound of Formula I, or a pharmaceutically acceptable salt thereof, described herein.

Further provided herein are methods of treating renal cell carcinoma by administering a therapeutically effective amount of a combination described herein where the combination includes a compound of Formula I, or a pharmaceutically acceptable salt thereof, and an anti-PD-1 antibody selected from nivolumab, pembrolizumab, pidilizumab, REGN2810 (also known as SAR-439684), PDR 001, SHR-1210 or MEDI0680. In another aspect the method includes treating renal cell carcinoma by administering AMP-224 in combination with a compound of Formula I, or a pharmaceutically acceptable salt thereof, described herein. In some embodiments, the renal cell carcinoma is a Stage I cancer. In some embodiments, the renal cell carcinoma is a Stage II cancer. In some embodiments, the renal cell carcinoma is a Stage III cancer. In some embodiments, the renal cell carcinoma is a Stage IV cancer. In some embodiments, the method is a method of treating Stage I renal cell carcinoma by administering a therapeutically effective amount of a combination described herein where the combination includes a compound of Formula I, or a pharmaceutically acceptable salt thereof, and an anti-PD-1 antibody selected from nivolumab, pembrolizumab, pidilizumab, REGN2810 (also known as SAR-439684), PDR 001, SHR-1210 or MEDI0680. In another aspect the method includes treating Stage I renal cell carcinoma by administering AMP-224 in combination with a compound of Formula I, or a pharmaceutically acceptable salt thereof, described herein. In another aspect the method is a method of treating Stage II renal cell carcinoma by administering a therapeutically effective amount of a combination described herein where the combination includes a compound of Formula I, or a pharmaceutically acceptable salt thereof, and an anti-PD-1 antibody selected from nivolumab, pembrolizumab, pidilizumab, REGN2810 (also known as SAR-439684), PDR 001, SHR-1210 or MEDI0680.

In another aspect the method includes treating Stage II renal cell carcinoma by administering AMP-224 in combination with a compound of Formula I, or a pharmaceutically acceptable salt thereof, described herein. In some embodiments, the method is a method of treating Stage III renal cell carcinoma by administering a therapeutically effective amount of a combination described herein where the combination includes a compound of Formula I, or a pharmaceutically acceptable salt thereof, and an anti-PD-1 antibody selected from nivolumab, pembrolizumab, pidilizumab, REGN2810 (also known as SAR-439684), PDR 001, SHR-1210 or MEDI0680. In another aspect the method includes treating Stage III renal cell carcinoma by administering AMP-224 in combination with a compound of Formula I, or a pharmaceutically acceptable salt thereof, described herein. Some embodiments include a method of treating Stage IV renal cell carcinoma by administering a therapeutically effective amount of a combination described herein where the combination includes a compound of Formula I, or a pharmaceutically acceptable salt thereof, and an anti-PD-1 antibody selected from nivolumab, pembrolizumab, pidilizumab, REGN2810 (also known as SAR-439684), PDR 001, SHR-1210 or MEDI0680. In another aspect the method includes treating Stage IV renal cell carcinoma by administering AMP-224 in combination with a compound of Formula I, or a pharmaceutically acceptable salt thereof, described herein.

Methods of treating colorectal cancer by administering a therapeutically effective amount of a combination described herein where the combination includes a compound of Formula I, or a pharmaceutically acceptable salt thereof, and an anti-PD-1 antibody selected from nivolumab, pembrolizumab, pidilizumab, REGN2810 (also known as SAR-439684), PDR 001, SHR-1210 or MEDIO680 are also provided herein. In another aspect the method includes treating colorectal cancer by administering AMP-224 in combination with a compound of Formula I, or a pharmaceutically acceptable salt thereof, described herein. In some embodiments, the colorectal cancer is a Stage I cancer. In some embodiments, the colorectal cancer is a Stage IIA, Stage JIB, or Stage IIC cancer. In some embodiments, the colorectal cancer is a Stage IIIA, Stage IIIB, or Stage IIIC cancer. In some embodiments, the colorectal cancer is a Stage IVA or Stage IVB cancer. In certain instances the colorectal cancer is further characterized by the grade of the cancer. The colorectal cancer can be a Grade 1, Grade 2, Grade 3, or Grade 4 cancer in any of the stages provided herein. In some embodiments, the method is a method of treating Stage I colorectal cancer by administering a therapeutically effective amount of a combination described herein where the combination includes a compound of Formula I, or a pharmaceutically acceptable salt thereof, and an anti-PD-1 antibody selected from nivolumab, pembrolizumab, pidilizumab, REGN2810 (also known as SAR-439684), PDR 001, SHR-1210 or MEDI0680. In another aspect the method includes treating Stage I colorectal cancer by administering AMP-224 in combination with a compound of Formula I, or a pharmaceutically acceptable salt thereof, described herein. In another aspect the method is a method of treating Stage II (e.g., Stage IIA, IIB, or IIC) colorectal cancer by administering a therapeutically effective amount of a combination described herein where the combination includes a compound of Formula I, or a pharmaceutically acceptable salt thereof, and an anti-PD-1 antibody selected from nivolumab, pembrolizumab, pidilizumab, REGN2810 (also known as SAR-439684), PDR 001, SHR-1210 or MEDI0680. In another aspect the method includes treating Stage II (e.g., Stage IIA, IIB, or IIC) colorectal cancer by administering AMP-224 in combination with a compound of Formula I, or a pharmaceutically acceptable salt thereof, described herein. In some embodiments, the method is a method of treating Stage III (e.g., Stage IIIA, IIIB, or IIIC) colorectal cancer by administering a therapeutically effective amount of a combination described herein where the combination includes a compound of Formula I, or a pharmaceutically acceptable salt thereof, and an anti-PD-1 antibody selected from nivolumab, pembrolizumab, pidilizumab, REGN2810 (also known as SAR-439684), PDR 001, SHR-1210 or MEDI0680. In another aspect the method includes treating Stage III (e.g., Stage IIIA, IIIB, or IIIC) colorectal cancer by administering AMP-224 in combination with a compound of Formula I described herein. Some embodiments include a method of treating Stage N (e.g., Stage NA or NB) colorectal cancer by administering a therapeutically effective amount of a combination described herein where the combination includes a compound of Formula I, or a pharmaceutically acceptable salt thereof, and an anti-PD-1 antibody selected from nivolumab, pembrolizumab, pidilizumab, REGN2810 (also known as SAR-439684), PDR 001, SHR-1210 or MEDI0680. In another aspect the method includes treating Stage IV (e.g., Stage NA or NB) colorectal cancer by administering AMP-224 in combination with a compound of Formula I, or a pharmaceutically acceptable salt thereof, described herein.

In other embodiments, the cancer is a hematological cancer selected from lymphoma, Non-Hodgkin lymphoma (NHL), Hodgkin's Lymphoma, Reed-Sternberg disease, multiple myeloma (MM), acute myelogenous leukemia (AML), chronic myelogenous leukemia (CMIL), acute lymphocytic leukemia, (ALL), or chronic lymphocytic leukemia (CLL). In certain embodiments, the cancer is Hodgkin's Lymphoma or Reed-Sternberg disease.

In certain embodiments, the methods of treating cancer include methods of treating NHL by administering a therapeutically effective amount of a combination described herein where the combination includes a compound of Formula I, or a pharmaceutically acceptable salt thereof, and an anti-PD-1 antibody selected from nivolumab, pembrolizumab, pidilizumab, REGN2810 (also known as SAR-439684), PDR 001, SHR-1210 or MEDI0680. In another aspect the method includes treating NHL by administering AMP-224 in combination with a compound of Formula I, or a pharmaceutically acceptable salt thereof, described herein. The NHL can be characterized by its stage according to, for example, the Ann Arbor staging system. The NHL can be indolent NHL (e.g., follicular lymphoma (FL); lymphoplasmacytic lymphoma (LL); marginal zone lymphoma (MZL) or Primary cutaneous anaplastic large cell lymphoma) or aggressive NHL (e.g., Diffuse large B-cell lymphoma (DLBCL); Follicular large cell lymphoma stage III; anaplastic large cell lymphoma; extranodal NK-tr-cell lymphoma; lymphomatoid granulmatosis; angioimmunoblastic T-cell lymphoma; peripheral T-cell lymphoma; intravascular large B-cell lymphoma; Burkitt lymphoma; lymphoblastic lymphoma; adult T-cell leukemia/lymphoma; or mantle cell lymphoma). In some embodiments, the NHL is a Stage I (e.g., Stage I(I) (thymus) or Stage I(E) (lymph system)) cancer. In some embodiments, the NHL is a Stage II (e.g., Stage II(I) (lymph nodes) or Stage II(E) (nearby organs)) cancer. In some embodiments, the NHL is a Stage III (e.g., Stage III(I) (lymph nodes), Stage III(E) (nearby organs), Stage III(S) (spleen), or Stage 111(ES) (nearby organs and spleen)) cancer. In some embodiments, the NHL is a Stage IV cancer. In some embodiments, the method is a method of treating Stage I (e.g., Stage 1(1) or 1(E)) NHL by administering a therapeutically effective amount of a combination described herein where the combination includes a compound of Formula I, or a pharmaceutically acceptable salt thereof, and an anti-PD-1 antibody selected from nivolumab, pembrolizumab, pidilizumab, REGN2810 (also known as SAR-439684), PDR 001, SHR-1210 or MEDI0680. In another aspect the method includes treating Stage I (e.g., Stage I(I) or I(E)) NHL by administering AMP-224 in combination with a compound of Formula I, or a pharmaceutically acceptable salt thereof, described herein. In another aspect the method is a method of treating Stage II (e.g., Stage 11(1) or II(E)) NHL by administering a therapeutically effective amount of a combination described herein where the combination includes a compound of Formula I, or a pharmaceutically acceptable salt thereof, and an anti-PD-1 antibody selected from nivolumab, pembrolizumab, pidilizumab, REGN2810 (also known as SAR-439684), PDR 001, SHR-1210 or MEDI0680. In another aspect the method includes treating Stage II (e.g., Stage II(I) or II(E)) NHL by administering AMP-224 in combination with a compound of Formula I, or a pharmaceutically acceptable salt thereof, described herein. In some embodiments, the method is a method of treating Stage III (e.g., Stage III(I), III(E), 111(S), or III(ES)) NHL by administering a therapeutically effective amount of a combination described herein where the combination includes a compound of Formula I, or a pharmaceutically acceptable salt thereof, and an anti-PD-1 antibody selected from nivolumab, pembrolizumab, pidilizumab, REGN2810 (also known as SAR-439684), PDR 001, SHR-1210 or MEDI0680. In another aspect the method includes treating Stage III (e.g., Stage III(I), III(E), III(S), or III(ES)) NHL by administering AMP-224 in combination with a compound of Formula I, or a pharmaceutically acceptable salt thereof, described herein. Some embodiments include a method of treating Stage IV NHL by administering a therapeutically effective amount of a combination described herein where the combination includes a compound of Formula I, or a pharmaceutically acceptable salt thereof, and an anti-PD-1 antibody selected from nivolumab, pembrolizumab, pidilizumab, REGN2810 (also known as SAR-439684), PDR 001, SHR-1210 or MEDI0680. In another aspect the method includes treating Stage IV NHL by administering AMP-224 in combination with a compound of Formula I, or a pharmaceutically acceptable salt thereof, described herein.

In another aspect are methods of treating Hodgkin's Lymphoma by administering a therapeutically effective amount of a combination described herein where the combination includes a compound of Formula I, or a pharmaceutically acceptable salt thereof, and an anti-PD-1 antibody selected from nivolumab, pembrolizumab, pidilizumab, REGN2810 (also known as SAR-439684), PDR 001, SHR-1210 or MEDI0680. In another aspect the method includes treating Hodgkin's Lymphoma by administering AMP-224 in combination with a compound of Formula I, or a pharmaceutically acceptable salt thereof, described herein. The Hodgkin's lymphoma can be classical or nodular lymphocyte-predominant. In some embodiments, the Hodgkin's Lymphoma includes Reed-Sternberg cells and can cause Reed-Sternberg disease. In some embodiments, the Hodgkin's Lymphoma is a Stage I cancer. In some embodiments, the Hodgkin's Lymphoma is a Stage II cancer. In some embodiments, the Hodgkin's Lymphoma is a Stage III cancer. In some embodiments, the Hodgkin's Lymphoma is a Stage IV cancer. In some embodiments, the method is a method of treating Stage I Hodgkin's Lymphoma by administering a therapeutically effective amount of a combination described herein where the combination includes a compound of Formula I, or a pharmaceutically acceptable salt thereof, and an anti-PD-1 antibody selected from nivolumab, pembrolizumab, pidilizumab, REGN2810 (also known as SAR-439684), PDR 001, SHR-1210 or MEDI0680. In another aspect the method includes treating Stage I Hodgkin's Lymphoma by administering AMP-224 in combination with a compound of Formula I, or a pharmaceutically acceptable salt thereof, described herein. In another aspect the method is a method of treating Stage II Hodgkin's Lymphoma by administering a therapeutically effective amount of a combination described herein where the combination includes a compound of Formula I, or a pharmaceutically acceptable salt thereof, and an anti-PD-1 antibody selected from nivolumab, pembrolizumab, pidilizumab, REGN2810 (also known as SAR-439684), PDR 001, SHR-1210 or MEDI0680. In another aspect the method includes treating Stage II Hodgkin's Lymphoma by administering AMP-224 in combination with a compound of Formula I, or a pharmaceutically acceptable salt thereof, described herein. In some embodiments, the method is a method of treating Stage III Hodgkin's Lymphoma by administering a therapeutically effective amount of a combination described herein where the combination includes a compound of Formula I, or a pharmaceutically acceptable salt thereof, and a PD-I antibody selected from nivolumab, pembrolizumab, pidilizumab, REGN2810 (also known as SAR-439684), PDR 001, SHR-1210 or MEDI0680. In another aspect the method includes treating Stage III Hodgkin's Lymphoma by administering AMP-224 in combination with a compound of Formula I, or a pharmaceutically acceptable salt thereof, described herein. Some embodiments include a method of treating Stage IV Hodgkin's Lymphoma by administering a therapeutically effective amount of a combination described herein where the combination includes a compound of Formula I, or a pharmaceutically acceptable salt thereof, and an anti-PD-1 antibody selected from nivolumab, pembrolizumab, pidilizumab, REGN2810 (also known as SAR-439684), PDR 001, SHR-1210 or MEDI0680. In another aspect the method includes treating Stage IV Hodgkin's Lymphoma by administering AMP-224 in combination with a compound of Formula I, or a pharmaceutically acceptable salt thereof, described herein.

Some embodiments are methods of treating chronic lymphocytic leukemia (CLL) by administering a therapeutically effective amount of a combination described herein where the combination includes a compound of Formula I, or a pharmaceutically acceptable salt thereof, and an anti-PD-1 antibody selected from nivolumab, pembrolizumab, pidilizumab, REGN2810 (also known as SAR-439684), PDR 001, SHR-1210 or MEDI0680. In some embodiments, the method includes treating CLL by administering AMP-224 in combination with a compound of Formula I, or a pharmaceutically acceptable salt thereof, described herein: CLL can be staged according to the Rai system or Binet System. For example, in some embodiments, the CLL is a Rai Stage I cancer. In some embodiments, the CLL is a Rai Stage II cancer. In some embodiments, the CLL is a Rai Stage III cancer. In some embodiments, the CLL is a Rai Stage IV cancer. In some embodiments, the CLL is a Binet Stage A cancer. The CLL can be a Binet Stage B cancer. The CLL can be a Binet Stage C cancer. In some embodiments, the method is a method of treating Rai Stage I CLL by administering a therapeutically effective amount of a combination described herein where the combination includes a compound of Formula I, or a pharmaceutically acceptable salt thereof, and an anti-PD-1 antibody selected from nivolumab, pembrolizumab, pidilizumab, REGN2810 (also known as SAR-439684), PDR 001, SHR-1210 or MEDI0680. In some embodiments, the method includes treating Rai Stage I CLL by administering AMP-224 in combination with a compound of Formula I, or a pharmaceutically acceptable salt thereof, described herein. In some embodiments, the method is a method of treating Rai Stage II CLL by administering a therapeutically effective amount of a combination described herein where the combination includes a compound of Formula I, or a pharmaceutically acceptable salt thereof, and an anti-PD-1 antibody selected from nivolumab, pembrolizumab, pidilizumab, REGN2810 (also known as SAR-439684), PDR 001, SHR-1210 or MEDI0680. In some embodiments, the method includes treating Rai Stage II CLL by administering AMP-224 in combination with a compound of Formula I, or a pharmaceutically acceptable salt thereof, described herein. In some embodiments, the method is a method of treating Rai Stage III CLL by administering a therapeutically effective amount of a combination described herein where the combination includes a compound of Formula I, or a pharmaceutically acceptable salt thereof, and an anti-PD-1 antibody selected from nivolumab, pembrolizumab, pidilizumab, REGN2810 (also known as SAR-439684), PDR 001, SHR-1210 or MEDI0680. In some embodiments, the method includes treating Rai Stage III CLL by administering AMP-224 in combination with a compound of Formula I, or a pharmaceutically acceptable salt thereof, described herein. In some embodiments, the method is a method of treating Rai Stage IV CLL by administering a therapeutically effective amount of a combination described herein where the combination includes a compound of Formula I, or a pharmaceutically acceptable salt thereof, and an anti-PD-1 antibody selected from nivolumab, pembrolizumab, pidilizumab, REGN2810 (also known as SAR-439684), PDR 001, SHR-1210 or MEDI0680. In some embodiments, the method includes treating Rai Stage IV CLL by administering AMP-224 in combination with a compound of Formula I, or a pharmaceutically acceptable salt thereof, described herein. In some embodiments, the method is a method of treating Binet Stage A CLL by administering a therapeutically effective amount of a combination described herein where the combination includes a compound of Formula I, or a pharmaceutically acceptable salt thereof, and an anti-PD-1 antibody selected from nivolumab, pembrolizumab, pidilizumab, REGN2810 (also known as SAR-439684), PDR 001, SHR-1210 or MEDI0680. In some embodiments, the method includes treating Binet Stage A CLL by administering AMP-224 in combination with a compound of Formula I, or a pharmaceutically acceptable salt thereof, described herein. In some embodiments, the method is a method of treating Binet Stage B CLL by administering a therapeutically effective amount of a combination described herein where the combination includes a compound of Formula I, or a pharmaceutically acceptable salt thereof, and an anti-PD-1 antibody selected from nivolumab, pembrolizumab, pidilizumab, REGN2810 (also known as SAR-439684), PDR 001, SHR-1210 or MEDI0680. In some embodiments, the method includes treating Binet Stage B CLL by administering AMP-224 in combination with a compound of Formula I, or a pharmaceutically acceptable salt thereof, described herein. In some embodiments, the method is a method of treating Binet Stage C CLL by administering a therapeutically effective amount of a combination described herein where the combination includes a compound of Formula I, or a pharmaceutically acceptable salt thereof, and an anti-PD-1 antibody selected from nivolumab, pembrolizumab, pidilizumab, REGN2810 (also known as SAR-439684), PDR 001, SHR-1210 or MEDI0680. In some embodiments, the method includes treating Binet Stage C CLL by administering AMP-224 in combination with a compound of Formula I, or a pharmaceutically acceptable salt thereof, described herein.

In some embodiments, there are provided methods of treating acute lymphoblastic leukemia (ALL) by administering a therapeutically effective amount of a combination described herein where the combination includes a compound of Formula I, or a pharmaceutically acceptable salt thereof, and an anti-PD-1 antibody selected from nivolumab, pembrolizumab, pidilizumab, REGN2810 (also known as SAR-439684), PDR 001, SHR-1210 or MEDI0680. In some embodiments, the method includes treating ALL by administering AMP-224 in combination with a compound of Formula I, or a pharmaceutically acceptable salt thereof, described herein. The ALL can be characterized according to the World Health Organization (WHO) classification. The ALL can be T-cell lymphoblastic leukemia. The ALL can be B-cell lymphoblastic leukemia. The ALL can be B-cell lymphoblastic leukemia having a recurrent genetic abnormality selected from: B lymphoblastic leukemia/lymphoma with t(9;22)(q34;ql1.2), BCR-ABLl; B lymphoblastic leukemia/lymphoma with t(v;llq23); MLL rearranged; B lymphoblastic leukemia/lymphoma with t(12;21)(p13;q22) TEL-AMLI (ETV6-RUNX1); B lymphoblastic leukemia/lymphoma with hyperdiploidy; B lymphoblastic leukemia/lymphoma with hypodiploidy; B lymphoblastic leukemia/lymphoma with t(5;14)(q31;q32) IL3-IGH; or B lymphoblastic leukemia/lymphoma with t(1;19)(q23;p13.3) TCF3-PBX1.

Some embodiments are methods of treating chronic myelogenous leukemia (CML) by administering a therapeutically effective amount of a combination described herein where the combination includes a compound of Formula I, or a pharmaceutically acceptable salt thereof, and an anti-PD-1 antibody selected from nivolumab, pembrolizumab, pidilizumab, REGN2810 (also known as SAR-439684), PDR 001, SHR-1210 or MEDI0680. In some embodiments, the method includes treating CML by administering AMP-224 in combination with a compound of Formula I, or a pharmaceutically acceptable salt thereof, described herein. The CML can be characterized by the phase of the disease. In some embodiments, the CML is in chronic phase (e.g., the patient has about less than 10% blasts in their blood or bone marrow). In some embodiments, the CML is in accelerated phase (e.g., the patient has (1) more than 10% blasts but fewer than 20% blasts in their blood or bone marrow; (2) basophil counts comprising at least about 20% of the white blood cell (WBC) count; (3) high WBC counts; (4) high or low platelet counts; or (5) chromosomal changes in the leukemia cells). In some embodiments, the CML is in blast phase (e.g., the patient has greater than 20% blasts in their blood or bone marrow). In some embodiments, the method is a method of treating CML in the chronic phase by administering a therapeutically effective amount of a combination described herein where the combination includes a compound of Formula I, or a pharmaceutically acceptable salt thereof, and an anti-PD-1 antibody selected from nivolumab, pembrolizumab, pidilizumab, REGN2810 (also known as SAR-439684), PDR 001, SHR-1210 or MEDI0680. In some embodiments, the method includes treating CML in the chronic phase by administering AMP-224 in combination with a compound of Formula I, or a pharmaceutically acceptable salt thereof, described herein. In some embodiments, the method is a method of treating CML in the accelerated phase by administering a therapeutically effective amount of a combination described herein where the combination includes a compound of Formula I, or a pharmaceutically acceptable salt thereof, and an anti-PD-1 antibody selected from nivolumab, pembrolizumab, pidilizumab, REGN2810 (also known as SAR-439684), PDR 001, SHR-1210 or MEDI0680. In some embodiments, the method includes treating CML in the accelerated phase by administering AMP-224 in combination with a compound of Formula I, or a pharmaceutically acceptable salt thereof, described herein. In some embodiments, the method is a method of treating CML in the blast phase by administering a therapeutically effective amount of a combination described herein where the combination includes a compound of Formula I, or a pharmaceutically acceptable salt thereof, and an anti-PD-1 antibody selected from nivolumab, pembrolizumab, pidilizumab, REGN2810 (also known as SAR-439684), PDR 001, SHR-1210 or MEDI0680. In some embodiments, the method includes treating CML in the blast phase by administering AMP-224 in combination with a compound of Formula I, or a pharmaceutically acceptable salt thereof, described herein.

Also provided herein are methods of treating AML by administering a therapeutically effective amount of a combination described herein where the combination includes a compound of Formula I, or a pharmaceutically acceptable salt thereof, and an anti-PD-1 antibody selected from nivolumab, pembrolizumab, pidilizumab, REGN2810 (also known as SAR-439684), PDR 001, SHR-1210 or MEDI0680. In some embodiments, the method includes treating AML by administering AMP-224 in combination with a compound of Formula I, or a pharmaceutically acceptable salt thereof, described herein. The AML can be characterized by, for example, the WHO classification system. In some embodiments, the AML is characterized by having certain genetic abnormalities including those provided below: AML with a translocation between chromosomes 8 and 21; AML with a translocation or inversion in chromosome 16; AML with a translocation between chromosomes 9 and 11; APL (M3) with a translocation between chromosomes 15 and 17; AML with a translocation between chromosomes 6 and 9; AML with a translocation or inversion in chromosome 3; or AML (megakaryoblastic) with a translocation between chromosomes 1 and 22.

The AML can be characterized as having myelodysplasia-related changes. The AML can be characterized as being related to previous anti-cancer therapy (e.g., chemotherapy or radiotherapy). The AML can be characterized as AML that is considered to not fall in the WHO groups above and includes, for example: AML with minimal differentiation (MO); AML without maturation (M1); AML with maturation (M2); Acute myelomonocytic leukemia (M4); Acute monocytic leukemia (MS); Acute erythroid leukemia (M6); Acute megakaryoblastic leukemia (M7); Acute basophilic leukemia; or Acute panmyelosis with fibrosis.

The combinations described herein can be administered to a cancer patient at any time following diagnosis. In some embodiments, the cancer patient can be treatment naive (i.e., has not received a cancer therapy for the diagnosed cancer). The cancer patient can be treatment naive for one cancer but can be diagnosed with one or more other cancers resulting from, for example, metastasis or malignancy. The cancer patient can be immune checkpoint naive for one or more cancers. The cancer patient can have a cancer that is refractory. In certain instances, the combinations described herein are administered as a first line therapy (e.g., the first therapy administered to a treatment naive cancer patient) to a patient in need thereof.

However, cancer morbidity and mortality is often associated with ineffective therapy or a cancer gaining resistant to or becoming refractory to one or more cancer therapies. The combinations described herein can, therefore, be administered to patients in need thereof as a second, third, fourth, fifth, sixth, or more line of treatment. The combinations described herein can be administered to a cancer patient who has been treated with at least one anti-cancer therapy or anti-cancer agent. In certain instances the patient has received at least one anti-cancer therapy including, for example, chemotherapy, radiotherapy, surgery, targeted therapy, immunotherapy, or a combination thereof. The patient can have a cancer that is resistant/refractory to treatment with at least one anti-cancer agent.

The methods of treating cancers herein include treating subjects who have been treated with a PD-L1 checkpoint inhibitor and have experienced no response to treatment, or a partial response, or stable disease, but then develop resistance to treatment with progression of disease or who have experienced a complete response to treatment, but then develop resistance to treatment with progression of disease (as defined by RECIST or other criteria). Resistance is defined as disease progression during treatment or a lack of response to treatment. Such PD-L1 inhibitor antibody treatment failures can be treated with PD-1 in combination with a WDR5 inhibitor.

### Response Criteria

RECIST: RECIST is a set of established criteria or standards, internationally recognized for evaluating patient response, stability and progression in clinical trials and in the clinical practice. Originally published in 2000, and revised in 2009 (Eisenhauer EA, et al.; New response criteria in solid tumors: revised RECIST guideline (version 1.1); Eur J Cancer 2009; 45:228-47), as a joint effort of the European Organization for Research and Treatment of Cancer, the National Cancer Institute of the United States and the National Cancer Institute of Canada Clinical Trials Group, RECIST has traditionally been utilized in the evaluation of response to chemotherapy.

Evaluation of target lesions: Complete Response (CR): Disappearance of all target lesions; Partial Response (PR): At least a 30% decrease in the sum of the LD (longest diameter) of target lesions, taking as reference the baseline sum LD; Stable Disease (SD): Neither sufficient shrinkage to qualify for PR nor sufficient increase to qualify for PD, taking as reference the smallest sum LD since the treatment started; Progressive Disease (PD): At least a 20% increase in the sum of the LD of target lesions, taking as reference the smallest sum LD recorded since the treatment started or the appearance of one or more new lesions.

Evaluation of non-target lesions

Complete Response (CR): Disappearance of all non-target lesions and normalization of tumor marker level; Incomplete Response/Stable Disease (SD): Persistence of one or more non-target lesion(s) or/and maintenance of tumor marker level above the normal limits; Progressive Disease (PD): Appearance of one or more new lesions and/or unequivocal progression of existing non-target lesions.

### Other Response Criteria

Other response criteria include the Immune-Related Response Criteria or iRECIST, as defined by Wolchok et al., in 2009 (Wolchok JD, et al.; Guidelines for the Evaluation of Immune Therapy Activity in Solid Tumors: Immune-Related Response Criteria. Clin. Cancer Res 2009; 15(23):7412-20) and the revised International Working Group Response Criteria (Cheson BD et al. Revised response criteria for malignant lymphoma. J. Clin. Oncol. 2007; 25:579-586).

The methods of treating cancer include methods for inhibiting cell growth by administering a therapeutically effective amount of a combination described herein where the combination includes a compound of Formula I, or a pharmaceutically acceptable salt thereof, and a PD-1 inhibitor described herein. In one example, the PD-1 inhibitor is AMP-224. Some embodiments include a method for inhibiting cell growth by administering a therapeutically effective amount of a combination described herein where the combination includes a compound of Formula I, or a pharmaceutically acceptable salt thereof, and an anti-PD-1 antibody selected from nivolumab, pembrolizumab, pidilizumab, REGN2810 (also known as SAR-439684), PDR 001, SHR-1210 or MEDI0680.

Also provided herein are methods of inhibiting metastasis of a cancer in a patient in need thereby by administering a therapeutically effective amount of a combination described herein where the combination includes a compound of Formula I, or a pharmaceutically acceptable salt thereof, and a PD-1 inhibitor described herein. In one example the PD-1 inhibitor is AMP-224. In another example is a method of inhibiting metastasis of a cancer in a patient in need thereby by administering a therapeutically effective amount of a combination described herein where the combination includes a compound of Formula I, or a pharmaceutically acceptable salt thereof, and an anti-PD-1 antibody selected from nivolumab, pembrolizumab, pidilizumab, REGN2810 (also known as SAR-439684), PDR 001, SHR-1210 or MEDI0680. In some embodiments, metastasis is inhibited by at least about 5%, 10%, 20%, 30%, 40%, 50%, 60%, 70%, 80%, 90%, or 100%.

In some embodiments, is a method of reducing pre-existing tumor metastasis in a cancer patient in need thereof by administering a therapeutically effective amount of a combination described herein where the combination includes a compound of Formula I, or a pharmaceutically acceptable salt thereof, and a PD-1 inhibitor described herein. In one example the PD-1 inhibitor is AMP-224. In another example is a method of reducing pre-existing tumor metastasis in a cancer patient in need thereof by administering a therapeutically effective amount of a combination described herein where the combination includes a compound of Formula I, or a pharmaceutically acceptable salt thereof, and an anti-PD-1 antibody selected from nivolumab, pembrolizumab, pidilizumab, REGN2810 (also known as SAR-439684), PDR 001, SHR-1210 or MEDI0680. In some embodiments, pre-existing tumor metastasis is reduced by at least about 5%, 10%, 20%, 30%, 40%, 50%, 60%, 70%, 80%, 90%, or 100%.

In some embodiments, the methods of treating cancer also provide for methods for reducing tumor burden in an individual by administering a therapeutically effective amount of a combination described herein where the combination includes a compound of Formula I, or a pharmaceutically acceptable salt thereof, and a PD-1 inhibitor described herein. In one example the PD-1 inhibitor is AMP-224. In another example is a method for reducing tumor burden in an individual by administering a therapeutically effective amount of a combination described herein where the combination includes a compound of Formula I, or a pharmaceutically acceptable salt thereof, and an anti-PD-1 antibody selected from n nivolumab, pembrolizumab, pidilizumab, REGN2810 (also known as SAR-439684), PDR 001, SHR-1210 or MEDI0680. In some embodiments, tumor burden is reduced by at least about 5%, 10%, 20%, 30%, 40%, 50%, 60%, 70%, 80%, 90%, or 100%.

In some embodiments, the methods of treating cancer also provide for methods for reducing tumor burden in a subject by administering a therapeutically effective amount of a combination described herein where the combination includes a compound of Formula I, or a pharmaceutically acceptable salt thereof, and a PD-1 inhibitor described herein. In one example the PD-1 inhibitor is AMP-224. In another example is a method for reducing tumor burden in an individual by administering a therapeutically effective amount of a combination described herein where the combination includes a compound of Formula I, or a pharmaceutically acceptable salt thereof, and an anti-PD-1 antibody selected from nivolumab, pembrolizumab, pidilizumab, REGN2810 (also known as SAR-439684), PDR 001, SHR-1210 or MEDI0680. In some embodiments, tumor burden is reduced by at least about 5%, 10%, 20%, 30%, 40%, 50%, 60%, 70%, 80%, 90%, or 100%.

The methods of treating cancer described herein also provide for methods for increasing or otherwise prolonging time to disease progression of certain stages (including advanced stages of cancer such as Stage III and IV cancer described herein). Time to disease progression can be prolonged in a patient by administering a therapeutically effective amount of a combination described herein where the combination includes a compound of Formula I, or a pharmaceutically acceptable salt thereof, and a PD-1 inhibitor described herein. In one example the PD-1 inhibitor is AMP-224. In another example is a method for increasing time to disease progression in a patient by administering a therapeutically effective amount of a combination described herein where the combination includes a compound of Formula I and an anti-PD-1 antibody selected from nivolumab, pembrolizumab, pidilizumab, REGN2810 (also known as SAR-439684), PDR 001, 5HR-1210 or MEDI0680. In some embodiments, the increase is a comparison between the time to disease progression without treatment and with treatment with a combination described herein. In some embodiments, the methods described herein prolong the time to disease progression by at least 1 week, 2 weeks, 3 weeks, 4 weeks, 1 month, 2 months, 3 months, 4 months, 5 months, 6 months, 7 months, 8 months, 9 months, 10 months, 11 months, 1 year, or more, including values therein.

The methods of treating cancer described herein also provide for methods for increasing or otherwise prolonging survival (including overall survival) of patients diagnosed with cancer as described herein. Patient survival can be prolonged by administering a therapeutically effective amount of a combination described herein where the combination includes a compound of Formula I, or a pharmaceutically acceptable salt thereof, and a PD-1 inhibitor described herein. In one example the PD-1 inhibitor is AMP-224. In another example is a method of prolonging patient survival by administering a therapeutically effective amount of a combination described herein where the combination includes a compound of Formula I, or a pharmaceutically acceptable salt thereof, and an anti-PD-1 antibody selected from nivolumab, pembrolizumab, pidilizumab, REGN2810 (also known as SAR-439684), PDR 001, SHR-1210 or MEDI0680. In some embodiments, the increase is a comparison between the survival without treatment and with treatment with a combination as described herein. In some embodiments, the methods described herein prolong survival by at least 1 week, 2 weeks, 3 weeks, 4 weeks, 1 month, 2 months, 3 months, 4 months, 5 months, 6 months, 7 months, 8 months, 9 months, 10 months, 11 months, 1 year, 2 years, or more, including values therein.

The methods of treating cancer described herein also provide for methods for increasing progression-free survival of patients diagnosed with cancer as described herein. Patient progression-free survival can be prolonged in a patient by administering a therapeutically effective amount of a combination described herein where the combination includes a compound of Formula I, or a pharmaceutically acceptable salt thereof, and a PD-1 inhibitor described herein. In one example the PD-1 inhibitor is AMP-224. In another example is a method for increasing progression-free survival of patients diagnosed with cancer by administering a therapeutically effective amount of a combination described herein where the combination includes a compound of Formula I, or a pharmaceutically acceptable salt thereof, and an anti-PD-1 antibody selected from nivolumab, pembrolizumab, pidilizumab, REGN2810 (also known as SAR-439684), PDR 001, SHR-1210 or MEDI0680. In some embodiments, the increase is a comparison between the progression-free survival without treatment and with treatment with a combination as described herein. In some embodiments, the methods described herein increase progression-free survival by at least 1 week, 2 weeks, 3 weeks, 4 weeks, 1 month, 2 months, 3 months, 4 months, 5 months, 6 months, 7 months, 8 months, 9 months, 10 months, 11 months, 1 year, 2 years, or more, including values therein.

Myeloid-derived suppressor cells (MDSC) have been shown to be tumorigenic, presumably due to their suppression of NK and killer T cell activity. Without being bound by theory, it is believed that inhibition of the interaction between WDR5 and MLL1 with a WDR5 inhibitor as described herein can reduce MDSC and thereby enhance the tumorigenicity of anti-tumor therapy with a PD-1 inhibitor. Thus, provided herein are methods of reducing a level of MDSC in a patient in need thereof by administering an effective amount of a combination described herein where the combination includes a compound of Formula I, or a pharmaceutically acceptable salt thereof, and a PD-1 inhibitor described herein. In some embodiments there is provided a method for reducing a level of MDSC in a patient in need thereof by administering an effective amount of a combination described herein where the combination includes a compound of Formula I, or a pharmaceutically acceptable salt thereof, and an anti-PD-1 antibody selected from nivolumab, pembrolizumab, pidilizumab, REGN2810 (also known as SAR-439684), PDR 001, SHR-1210 or MEDIO680 compared to administration of a compound of Formula I, or a pharmaceutically acceptable salt thereof, or a PD-1 inhibitor alone. The reduction of MDSC can benefit the treatment of a cancer described herein. The level of MDSC in a human patient can be measured before, during, and after administration of a combination described herein. In some embodiments, it can be useful to compare pre- and post-administration amounts of MDSC in the patient. A reduction in the amount, level, or number of MDSC following administration can indicate effectiveness of the combination in, for example, treating a cancer described herein. MDSC levels can be monitored over the course of a treatment or regimen described herein with a combination described herein. In such instances, the determination of MDSC levels at various points during the course of administration can indicate the effectiveness of the regimen.

Regulatory T cells (Treg cells) have been shown to be tumorigenic, presumably due to their suppression of NK and kller T cell activity. Without being bound by theory, it is believed that inhibition of the interaction between WDR5 and MLL1 with a WDR5 inhibitor as described herein can reduce Treg cells and thereby enhance the tumorigenicity of anti-tumor therapy with a PD-1 inhibitor. Thus, methods of reducing the percentage or level of Treg cells in a patient in need thereof are also provided herein. Such methods include administering an effective amount of a combination described herein where the combination includes a compound of Formula I, or a pharmaceutically acceptable salt thereof, and a PD-1 inhibitor described herein. In one example the PD-1 inhibitor is AMP-224. In some embodiments, there is provided a method of reducing the percentage or level of Treg cells in a patient in need thereof by administering an effective amount of a combination described herein where the combination includes a compound of Formula I, or a pharmaceutically acceptable salt thereof, and an anti-PD-1 antibody selected from nivolumab, pembrolizumab, pidilizumab, REGN2810 (also known as SAR-439684), PDR 001, SHR-1210 or MEDIO680 to the patient, wherein the administration decreases the percentage or level of Treg cells in the patient compared to the level prior to the administration. The reduction of Treg cells can benefit the treatment of a cancer described herein. The level of Treg cells in a human patient can be measured before, during, and after administration of a combination described herein. In some embodiments, it can be useful to compare pre- and post-administration amounts of Treg cells in the patient. A reduction in the amount, level, or number of Treg cells following administration can indicate effectiveness of the combination in, for example, treating a cancer described herein. Treg cell levels can be monitored over the course of a treatment or regimen described herein with a combination described herein. In such instances, the determination of Treg cells levels at various points during the course of administration can indicate the effectiveness of the regimen.

The combinations described herein can be useful in methods of enhancing activity of natural killer (NK) cells, directly and/or indirectly (e.g., through reduction of MDSC and/or Treg cells). Similarly, the combinations described herein can also be useful in methods of enhancing activity of cytotoxic T-cells. The methods of enhancing include contacting a NK cell or cytotoxic T-cell with a combination described herein where the combination enhances the activity of the NK cell or cytotoxic T-cell relative to its activity prior to the contact. In some embodiments, the enhanced activity of the NK cell or cytotoxic T-cell is in a cancer patient who has been administered a combination as described herein. Such combinations useful for enhancing activity of NK cells or cytotoxic T-cells can include AMP-224. In other examples, combinations described herein useful in methods for enhancing activity of NK cells or cytotoxic T-cells include a PD-1 selected from nivolumab, pembrolizumab, pidilizumab, REGN2810 (also known as SAR-439684), PDR 001, SHR-1210 or MEDI0680.

The combinations described herein can also enhance antibody-dependent cell-mediated cytotoxicity in a cancer patient upon administration of a combination as described herein.

The combinations described herein can include administration of each therapy (e.g., a compound of Formula I and a PD-1 inhibitor), where the administration is performed simultaneously or sequentially (in either order). In some embodiments, the compound of Formula I and the PD-1 inhibitor are administered simultaneously (e.g., within at least 1 to 5 min of each other). In some embodiments, the compound of Formula I and the PD-1 inhibitor are administered sequentially (e.g., within at least 10 min, 15 min, 30 min, 1 h, 2 h, 5 h, 10 h, 12 h, 1 day, 2 days, 5 days, 7 days, 14 days, or 21 days of each other).

In one example a compound of Formula I, or a pharmaceutically acceptable salt thereof, is administered concurrently with an anti-PD-1 antibody selected from nivolumab, pembrolizumab, pidilizumab, REGN2810 (also known as SAR-439684), PDR 001, SHR-1210, or MEDI0680. In another example, a compound of Formula I, or a pharmaceutically acceptable salt thereof, can be administered prior to the administration of nivolumab. In another example, a compound of Formula I, or a pharmaceutically acceptable salt thereof, can be administered prior to the administration of pembrolizumab. In another example, a compound of Formula I, or a pharmaceutically acceptable salt thereof, can be administered prior to the administration of pidilizumab. In another example, a compound of Formula I, or a pharmaceutically acceptable salt thereof, can be administered prior to the administration of REGN2810 (also known as SAR-439684). In some embodiments, a compound of Formula I, or a pharmaceutically acceptable salt thereof, can be administered prior to the administration of PDR 001. In some embodiments, a compound of Formula I, or a pharmaceutically acceptable salt thereof, can be administered prior to the administration of MEDI0680. In some embodiments, a compound of Formula I, or a pharmaceutically acceptable salt thereof, can be administered after the administration of nivolumab. In some embodiments, a compound of Formula I, or a pharmaceutically acceptable salt thereof, can be administered after the administration of pembrolizumab, atezolizumab or SHR-1210. In some embodiments, a compound of Formula I, or a pharmaceutically acceptable salt thereof, can be administered after the administration of pidilizumab, atezolizumab or SHR-1210. In some embodiments, a compound of Formula I, or a pharmaceutically acceptable salt thereof, can be administered after the administration of REGN2810 (also known as SAR-439684). In some embodiments, a compound of Formula I, or a pharmaceutically acceptable salt thereof, can be administered prior after administration of PDR 001. In some embodiments, a compound of Formula I, or a pharmaceutically acceptable salt thereof, can be administered after the administration of MEDI0680.

In some embodiments, a compound of Formula I, or a pharmaceutically acceptable salt thereof, is administered concurrently with AMP-224. In some embodiments, a compound of Formula I, or a pharmaceutically acceptable salt thereof, is administered prior to administration of AMP-224. In some embodiments, a compound of Formula I, or a pharmaceutically acceptable salt thereof, is administered after administration of AMP-224.

The compound of Formula I, or a pharmaceutically acceptable salt thereof, can be administered, for example, once a day (QD), twice daily (BID), once a week (QW), twice weekly (BIW), three times a week (TIW), or monthly (QM). In some embodiments, the compound of Formula I, or a pharmaceutically acceptable salt thereof, can be administered BID. The compound of Formula I, or a pharmaceutically acceptable salt thereof, can be administered TIW. In certain instances, the compound of Formula I, or a pharmaceutically acceptable salt thereof, is administered 2 to 3 times a week. In some embodiments, the compound of Formula I, or a pharmaceutically acceptable salt thereof, is administered QD. The compound can be administered QD for about: 1 day to about 7 days, 1 day to about 14 days, 1 day to about 21 days, 1 day to about 28 days, or daily until disease progression or unacceptable toxicity. The administration of a compound of Formula I, or a pharmaceutically acceptable salt thereof, can, in part, depend upon the tolerance of the patient where greater tolerance can allow greater or more frequent administration. Alternatively, where a patient shows poor tolerance to a compound of Formula I, a lesser amount of the compound or a less frequent dosing can be performed. The administration of compound can also cease when maximum treatment effect is achieved and then resume when further administration is warranted, albeit with an alternative schedule and dose. Compounds of Formula I can be administered in any regimen as described herein.

In some embodiments, a compound of Formula I, or a pharmaceutically acceptable salt thereof, can be administered to a patient at an amount (expressed in mg of the free base) of about: 1 mg, 2 mg, 3 mg, 4 mg, 5 mg, 10 mg, 15 mg, 20 mg, 25 mg, 30 mg, 35 mg, 40 mg, 45 mg, 50 mg, 60 mg, 70 mg, 80 mg, 85 mg, 90 mg, 100 mg, 125 mg, 150 mg, 175 mg, or 200 mg, QD. In some embodiments, a compound of Formula I, or a pharmaceutically acceptable salt thereof, can be administered at an amount of about: 1 mg, 2 mg, 3 mg, 4 mg, 5 mg, 10 mg, 15 mg, 20 mg, 25 mg, 30 mg, 35 mg, 40 mg, 45 mg, 50 mg, 60 mg, 70 mg, 80 mg, 85 mg, 90 mg, 100 mg, 125 mg, 150 mg, 175 mg, or 200 mg, BIW. In some embodiments, a compound of Formula I can be administered at an amount of about: 1 mg, 2 mg, 3 mg, 4 mg, 5 mg, 10 mg, 15 mg, 20 mg, 25 mg, 30 mg, 35 mg, 40 mg, 45 mg, 50 mg, 60 mg, 70 mg, 80 mg, 85 mg, 90 mg, 100 mg, 125 mg, 150 mg, 175 mg, or 200 mg, TIW. In some embodiments, a compound of Formula I can be administered at an amount of about: 1 mg, 2 mg, 3 mg, 4 mg, 5 mg, 10 mg, 15 mg, 20 mg, 25 mg, 30 mg, 35 mg, 40 mg, 45 mg, 50 mg, 60 mg, 70 mg, 80 mg, 85 mg, 90 mg, 100 mg, 125 mg, 150 mg, 175 mg, or 200 mg, QW. In some embodiments, a compound of Formula I can be administered at an amount of about: 1 mg, 2 mg, 3 mg, 4 mg, 5 mg, 10 mg, 15 mg, 20 mg, 25 mg, 30 mg, 35 mg, 40 mg, 45 mg, 50 mg, 60 mg, 70 mg, 80 mg, 85 mg, 90 mg, 100 mg, 125 mg, 150 mg, 175 mg, or 200 mg, Q2W. In some embodiments, a compound of Formula I can be administered at an amount of about 5 mg or about 10 mg, QD. In some embodiments, a compound of Formula I can be administered at an amount of about 5 mg or about 10 mg, BIW. In some embodiments, a compound of Formula I can be administered at an amount of about 5 mg or about 10 mg, TIW. In some embodiments, a compound of Formula I can be administered at an amount of about 5 mg or about 10 mg, QW. In some embodiments, a compound of Formula I can be administered at an amount of about 5 mg or about 10 mg, Q2W. Administration of a compound of Formula I, or a pharmaceutically acceptable salt thereof, can be continuous. Administration of a compound of Formula I, or a pharmaceutically acceptable salt thereof, can be intermittent.

In some embodiments, a compound of Formula I, or a pharmaceutically acceptable salt thereof, can be administered at an amount of about: 1 mg to about 10 mg, 1 mg to about 25 mg, 1 mg to about 50 mg, 5 mg to about 10 mg, 5 mg to about 25 mg, 5 mg to about 50 mg, 10 mg to about 25 mg, 10 mg to about 50 mg, 50 mg to about 100 mg, or 100 mg to about 200 mg, QD. In some embodiments, a compound of Formula I can be administered at an amount of about: 1 mg to about 10 mg, 1 mg to about 25 mg, 1 mg to about 50 mg, 5 mg to about 10 mg, 5 mg to about 25 mg, 5 mg to about 50 mg, 10 mg to about 25 mg, 10 mg to about 50 mg, 50 mg to about 100 mg, or 100 mg to about 200 mg, BIW. In some embodiments, a compound of Formula I, or a pharmaceutically acceptable salt thereof, can be administered at an amount of about: 1 mg to about 10 mg, 1 mg to about 25 mg, 1 mg to about 50 mg, 5 mg to about 10 mg, 5 mg to about 25 mg, 5 mg to about 50 mg, 10 mg to about 25 mg, 10 mg to about 50 mg, 50 mg to about 100 mg, or 100 mg to about 200 mg, TIW. In some embodiments, a compound of Formula I can be administered at an amount of about: 1 mg to about 10 mg, 1 mg to about 25 mg, 1 mg to about 50 mg, 5 mg to about 10 mg, 5 mg to about 25 mg, 5 mg to about 50 mg, 10 mg to about 25 mg, 10 mg to about 50 mg, 50 mg to about 100 mg, or 100 mg to about 200 mg, QW. In some embodiments, a compound of Formula I can be administered at an amount of about: 1 mg to about 10 mg, 1 mg to about 25 mg, 1 mg to about 50 mg, 5 mg to about 10 mg, 5 mg to about 25 mg, 5 mg to about 50 mg, 10 mg to about 25 mg, 10 mg to about 50 mg, 50 mg to about 100 mg, or 100 mg to about 200 mg, Q2W. Administration of a compound of Formula I, or a pharmaceutically acceptable salt thereof, can be continuous. Administration of a compound of Formula I, or a pharmaceutically acceptable salt thereof, can be intermittent.

In some embodiments, a compound of Formula I, or a pharmaceutically acceptable salt thereof, can be administered at an amount of about: 0.0001 mg/kg to about 200 mg/kg, 0.001 mg/kg to about 200 mg/kg, 0.01 mg/kg to about 200 mg/kg, 0.01 mg/kg to about 150 mg/kg, 0.01 mg/kg to about 100 mg/kg, 0.01 mg/kg to about 50 mg/kg, 0.01 mg/kg to about 25 mg/kg, 0.01 mg/kg to about 10 mg/kg, or 0.01 mg/kg to about 5 mg/kg, 0.05 mg/kg to about 200 mg/kg, 0.05 mg/kg to about 150 mg/kg, 0.05 mg/kg to about 100 mg/kg, 0.05 mg/kg to about 50 mg/kg, 0.05 mg/kg to about 25 mg/kg, 0.05 mg/kg to about 10 mg/kg, or 0.05 mg/kg to about 5 mg/kg, 0.5 mg/kg to about 200 mg/kg, 0.5 mg/kg to about 150 mg/kg, 0.5 mg/kg to about 100 mg/kg, 0.5 mg/kg to about 50 mg/kg, 0.5 mg/kg to about 25 mg/kg, 0.5 mg/kg to about 10 mg/kg, or 0.5 mg/kg to about 5 mg/kg, QD. In some embodiments, a compound of Formula I can be administered at an amount of about: 0.0001 mg/kg to about 200 mg/kg, 0.001 mg/kg to about 200 mg/kg, 0.5 mg/kg to about 200 mg/kg, 0.5 mg/kg to about 150 mg/kg, 0.5 mg/kg to about 100 mg/kg, 0.5 mg/kg to about 50 mg/kg, 0.5 mg/kg to about 25 mg/kg, 0.5 mg/kg to about 10 mg/kg, or 0.5 mg/kg to about 5 mg/kg, BIW. In some embodiments, a compound of Formula I can be administered at an amount of about: 0.0001 mg/kg to about 200 mg/kg, 0.001 mg/kg to about 200 mg/kg, 0.5 mg/kg to about 200 mg/kg, 0.5 mg/kg to about 150 mg/kg, 0.5 mg/kg to about 100 mg/kg, 0.5 mg/kg to about 50 mg/kg, 0.5 mg/kg to about 25 mg/kg, 0.5 mg/kg to about 10 mg/kg, or 0.5 mg/kg to about 5 mg/kg, TIW. In some embodiments, a compound of Formula I can be administered at an amount of about: 0.0001 mg/kg to about 200 mg/kg, 0.001 mg/kg to about 200 mg/kg, 0.5 mg/kg to about 200 mg/kg, 0.5 mg/kg to about 150 mg/kg, 0.5 mg/kg to about 100 mg/kg, 0.5 mg/kg to about 50 mg/kg, 0.5 mg/kg to about 25 mg/kg, 0.5 mg/kg to about 10 mg/kg, or 0.5 mg/kg to about 5 mg/kg, QW. In some embodiments, a compound of Formula I can be administered at an amount of about: 0.0001 mg/kg to about 200 mg/kg, 0.001 mg/kg to about 200 mg/kg, 0.5 mg/kg to about 200 mg/kg, 0.5 mg/kg to about 150 mg/kg, 0.5 mg/kg to about 100 mg/kg, 0.5 mg/kg to about 50 mg/kg, 0.5 mg/kg to about 25 mg/kg, 0.5 mg/kg to about 10 mg/kg, or 0.5 mg/kg to about 5 mg/kg, Q2W. Administration of a compound of Formula I can be continuous. Administration of a compound of Formula I can be intermittent.

In some embodiments, a compound of Formula I, or a pharmaceutically acceptable salt thereof, can be administered at an amount of about: 1 mg/kg to about 200 mg/kg, 1 mg/kg to about 150 mg/kg, 1 mg/kg to about 100 mg/kg, 1 mg/kg to about 50 mg/kg, 1 mg/kg to about 25 mg/kg, 1 mg/kg to about 10 mg/kg, or 1 mg/kg to about 5 mg/kg, QD. In some embodiments, a compound of Formula I can be administered at an amount of about: 1 mg/kg to about 200 mg/kg, 1 mg/kg to about 150 mg/kg, 1 mg/kg to about 100 mg/kg, 1 mg/kg to about 80 mg/kg, 1 mg/kg to about 50 mg/kg, 1 mg/kg to about 25 mg/kg, 1 mg/kg to about 10 mg/kg, or 1 mg/kg to about 5 mg/kg, BIW. In some embodiments, a compound of Formula I can be administered at an amount of about: 1 mg/kg to about 200 mg/kg, 1 mg/kg to about 150 mg/kg, 1 mg/kg to about 100 mg/kg, 1 mg/kg to about 50 mg/kg, 1 mg/kg to about 25 mg/kg, 1 mg/kg to about 10 mg/kg, or 1 mg/kg to about 5 mg/kg, TIW. In some embodiments, a compound of Formula I can be administered at an amount of about: 1 mg/kg to about 200 mg/kg, 1 mg/kg to about 150 mg/kg, 1 mg/kg to about 100 mg/kg, 1 mg/kg to about 50 mg/kg, 1 mg/kg to about 25 mg/kg, 1 mg/kg to about 10 mg/kg, or 1 mg/kg to about 5 mg/kg, QW. In some embodiments, a compound of Formula I can be administered at an amount of about: 1 mg/kg to about 200 mg/kg, 1 mg/kg to about 150 mg/kg, 1 mg/kg to about 100 mg/kg, 1 mg/kg to about 50 mg/kg, 1 mg/kg to about 25 mg/kg, 1 mg/kg to about 10 mg/kg, or 1 mg/kg to about 5 mg/kg, Q2W. In one example, a compound of Formula I can be administered at an amount of about 15 mg/kg to about 75 mg/kg, QD. In some embodiments, a compound of Formula I can be administered at an amount of about 20 mg/kg to about 50 mg/kg. In some embodiments, a compound of Formula I can be administered at an amount of about 0.001 mg/kg, 0.01 mg/kg, 0.05 mg/kg, 0.1 mg/kg, 0.5 mg/kg, 1 mg/kg, 2 mg/kg, 3 mg/kg, 4 mg/kg, 5 mg/kg, 10 mg/kg, 15 mg/kg, 20 mg/kg, 25 mg/kg, 30 mg/kg, 40 mg/kg, 50 mg/kg, 60 mg/kg, 70 mg/kg, 80 mg/kg, 90 mg/kg, 100 mg/kg, 125 mg/kg, 150 mg/kg, 175 mg/kg, or 200 mg/kg. Administration of a compound of Formula I can be continuous. In one example, a compound of Formula I can be administered at an amount of about 40 mg/kg to about 80 mg/kg, QD. Administration of a compound of Formula I can be intermittent.

As used herein, the term ‘daily’ is intended to mean that a therapeutic compound of a combination described herein, such as a compound of Formula I, is administered once or more than once each day for a period of time. The term ‘continuous’ is intended to mean that a therapeutic compound of a combination described herein, such as a compound of Formula I, is administered daily for an uninterrupted period of at least 10 days to 52 weeks. The term ‘intermittent’ or ‘intermittently’ as used herein is intended to mean stopping and starting at either regular or irregular intervals. In some embodiments, intermittent administration of a therapeutic compound of a combination described herein, such as a compound of Formula I, includes administration for one to six days per week (e.g., 2 to 3 times per week or QD), administration in cycles (e.g., daily administration for two to eight consecutive weeks, then a rest period with no administration at least one day), or, for example, administration on alternate days.

Where the PD-1 inhibitor is an anti-PD-1 antibody, it can be administered according to established regimens such as those provided in a package insert. The PD-I antibody can be administered in an amount described herein and can be administered QW, once every 2 weeks (Q2W), or once every 3 weeks (Q3W). In some embodiments, the PD-1 antibody is administered once every two or three weeks. In some embodiments, the PD-1 antibody is administered Q2W. In some embodiments, the PD-1 antibody is administered Q3W. In some embodiments, the PD-1 antibody is administered BIW for at least 3 weeks.

In some embodiments, nivolumab can be administered to a patient at an amount of about 0.1 to about 10 mg/kg (mg drug/kg body mass) (including, for example, 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg), QW. In some embodiments, nivolumab can be administered to a patient at an amount of about 0.1 to about 10 mg/kg (including, for example, 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg), Q2W. In some embodiments, nivolumab can be administered to a patient at an amount of about 0.1 to about 10 mg/kg (including, for example, 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg), Q4W. In some embodiments, nivolumab can be administered at an amount of about 0.1 to about 10 mg/kg (including, for example, 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg), B4W (twice every 4 weeks). In some embodiments, nivolumab can be administered to a patient at an amount of about 0.1 to about 10 mg/kg (including, for example, 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg), Q3W. In some embodiments, nivolumab can be administered at an amount of about 10 mg/kg, BIW. Administration of nivolumab can be continuous. Administration of nivolumab can be intermittent.

Nivolumab can be administered as an intravenous infusion over about 10, 20, 30, 40, 50, or 60 or more minutes. Nivolumab can be administered as an intravenous infusion over about 60 minutes once every 1, 2, 3, 4, 5 or more weeks. Nivolumab can be administered as an intravenous infusion over about 60 minutes once every two weeks. Nivolumab can be administered as an intravenous infusion over about 60 minutes once every three weeks. Nivolumab can be administered as an intravenous infusion over about 60 minutes once every four weeks. Nivolumab can be administered as an intravenous infusion according to a package insert. Administration of nivolumab can be continuous. Administration of nivolumab can be intermittent.

In some embodiments, pembrolizumab can be administered at an amount of about 0.5 to about 20 mg/kg (including, for example, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg). In some embodiments, pembrolizumab can be administered at an amount of about 0.5 to about 20 mg/kg (including, for example, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg) QW. In some embodiments, pembrolizumab can be administered at an amount of about 0.5 to about 20 mg/kg (including, for example, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg) Q2W. In some embodiments, pembrolizumab can be administered at an amount of about 0.5 to about 20 mg/kg (including, for example, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg) Q3W. In some embodiments, pembrolizumab can be administered at an amount of about 0.5 to about 20 mg/kg (including, for example, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg) Q4W. Administration of pembrolizumab can be continuous. Administration of pembrolizumab can be intermittent.

Pembrolizumab can be administered as an intravenous infusion over about 10, 20, 30, 40, 50, or 60 or more minutes. Pembrolizumab can be administered as an intravenous infusion over about 60 minutes once every 1, 2, 3, 4, 5 or more weeks. Pembrolizumab can be administered as an intravenous infusion over about 60 minutes once every two weeks. Pembrolizumab can be administered as an intravenous infusion over about 60 minutes once every three weeks. Pembrolizumab can be administered as an intravenous infusion over about 60 minutes once every four weeks. Pembrolizumab can be administered according to a provided package insert. Administration of pembrolizumab can be continuous. Administration of pembrolizumab can be intermittent.

In some embodiments, pidilizumab can be administered at an amount of about 0.1 to about 30 mg/kg (including, for example, 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg, 20 mg/kg, 25 mg/kg, 30 mg/kg), QW. In some embodiments, pidilizumab can be administered at an amount of about 0.1 to about 30 mg/kg (including, for example, 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg, 20 mg/kg, 25 mg/kg, 30 mg/kg), Q2W. In some embodiments, pidilizumab can be administered at an amount of about 0.1 to about 30 mg/kg (including, for example, 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg, 20 mg/kg, 25 mg/kg, 30 mg/kg), Q3W. In some embodiments, pidilizumab can be administered at an amount of about 0.1 to about 30 mg/kg (including, for example, 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg, 20 mg/kg, 25 mg/kg, 30 mg/kg), Q4W. Administration of pidilizumab can be continuous. Administration of pidilizumab can be intermittent.

Pidilizumab can be administered as an intravenous infusion over about 10, 20, 30, 40, 50, or 60 or more minutes. Pidilizumab can be administered as an intravenous infusion over about 60 minutes once every 1, 2, 3, 4, 5 or more weeks. Pidilizumab can be administered as an intravenous infusion over about 60 minutes once every two weeks. Pidilizumab can be administered as an intravenous infusion over about 60 minutes once every three weeks. Pidilizumab can be administered as an intravenous infusion over about 60 minutes once every four weeks. Administration of pidilizumab can be continuous. Administration of pidilizumab can be intermittent.

In some embodiments, AMP-224 can be administered at an amount of about 1 to about 50 mg/kg (including, for example, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg), QW. In some embodiments, AMP-224 can be administered at an amount of about 1 to about 50 mg/kg (including, for example, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg), Q2W. In some embodiments, AMP-224 can be administered (for example by subcutaneous administration) at an amount of about 1 to about 50 mg/kg (including, for example, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg), Q3W. In some embodiments, AMP-224 can be administered (for example by subcutaneous administration) at an amount of about 1 to about 50 mg/kg (including, for example, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg), Q4W. Administration of AMP-224 can be continuous. Administration of AMP-224 can be intermittent.

AMP-224 can be administered as an intravenous infusion over about 10, 20, 30, 40, 50, or 60 or more minutes. AMP-224 can be administered as an intravenous infusion over about 60 minutes once every 1, 2, 3, 4, 5 or more weeks. AMP-224 can be administered as an intravenous infusion over about 60 minutes once every two weeks. AMP-224 can be administered as an intravenous infusion over about 60 minutes twice every three weeks. AMP-224 can be administered as an intravenous infusion over about 60 minutes three times every six weeks. Administration of AMP-224 can be continuous. Administration of AMP-224 can be intermittent.

In some embodiments, REGN2810 (also known as SAR-439684) can be administered at an amount of about 0.1 to about 30 mg/kg (including, for example, 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg, 20 mg/kg, 25 mg/kg, 30 mg/kg), Q2W. In some embodiments, REGN2810 (also known as SAR-439684) can be administered at an amount of about 0.1 to about 30 mg/kg (including, for example, 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg, 20 mg/kg, 25 mg/kg, 30 mg/kg), Q4W. In some embodiments, REGN2810 (also known as SAR-439684) can be administered at an amount of about 0.1 to about 30 mg/kg (including, for example, 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg, 20 mg/kg, 25 mg/kg, 30 mg/kg), B4W. In some embodiments, REGN2810 (also known as SAR-439684) can be administered at an amount of about 0.1 to about 30 mg/kg (including, for example, 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg, 20 mg/kg, 25 mg/kg, 30 mg/kg), QW. Administration of REGN2810 (also known as SAR-439684) can be continuous. Administration of REGN2810 can be intermittent.

REGN2810 (also known as SAR-439684) can be administered as an intravenous infusion over about 10, 20, 30, 40, 50, or 60 or more minutes. REGN2810 (also known as SAR-439684) can be administered as an intravenous infusion over about 60 minutes once every 1, 2, 3, 4, 5 or more weeks. REGN2810 (also known as SAR-439684) can be administered as an intravenous infusion over about 60 minutes once every two weeks. REGN2810 (also known as SAR-439684) can be administered as an intravenous infusion over about 60 minutes twice every three weeks. REGN2810 (also known as SAR-439684) can be administered as an intravenous infusion over about 60 minutes three times every six weeks. Administration of REGN2810 (also known as SAR-439684) can be continuous. Administration of REGN2810 (also known as SAR-439684) can be intermittent.

In some embodiments, PDR 001 can be administered at an amount of about 0.5 to about 30 mg/kg (including, for example, 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg, 20 mg/kg, 25 mg/kg, 30 mg/kg), QW. In some embodiments, PDR 001 can be administered at an amount of about 0.5 to about 30 mg/kg (including, for example, 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg, 20 mg/kg, 25 mg/kg, 30 mg/kg), Q2W. In some embodiments, PDR 001 can be administered at an amount of about 0.5 to about 30 mg/kg (including, for example, 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg, 20 mg/kg, 25 mg/kg, 30 mg/kg), Q3W. In some embodiments, PDR 001 can be administered at an amount of about 0.5 to about 30 mg/kg (including, for example, 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg, 20 mg/kg, 25 mg/kg, 30 mg/kg), Q4W. Administration of PDR 001 can be continuous. Administration of PDR 001 can be intermittent.

PDR 001 can be administered as an intravenous infusion over about 10, 20, 30, 40, 50, or 60 or more minutes. PDR 001 can be administered as an intravenous infusion over about 60 minutes once every 1, 2, 3, 4, 5 or more weeks. PDR 001 can be administered as an intravenous infusion over about 60 minutes once every two weeks. PDR 001 can be administered as an intravenous infusion over about 60 minutes twice every three weeks. PDR 001 can be administered as an intravenous infusion over about 60 minutes once every three weeks. Administration of PDR 001 can be continuous. Administration of PDR 001 can be intermittent.

In some embodiments, MEDIO680 can be administered at an amount of about 0.5 to about 30 mg/kg (including, for example, 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg, 20 mg/kg, 25 mg/kg, 30 mg/kg), QW. In some embodiments, MEDI0680 can be administered at an amount of about 0.5 to about 30 mg/kg (including, for example, 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg, 20 mg/kg, 25 mg/kg, 30 mg/kg), Q2W. In some embodiments, MEDIO680 can be administered at an amount of about 0.5 to about 30 mg/kg (including, for example, 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg, 20 mg/kg, 25 mg/kg, 30 mg/kg), Q3W. In some embodiments, MEDIO680 can be administered at an amount of about 0.5 to about 30 mg/kg (including, for example, 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg, 20 mg/kg, 25 mg/kg, 30 mg/kg), Q4W. Administration of MEDIO680 can be continuous. Administration of MEDIO680 can be intermittent.

MEDIO680 can be administered as an intravenous infusion over about 10, 20, 30, 40, 50, or 60 or more minutes. MEDIO680 can be administered as an intravenous infusion over about 60 minutes once every 1, 2, 3, 4, 5 or more weeks. MEDIO680 can be administered as an intravenous infusion over about 60 minutes once every two weeks. MEDIO680 can be administered as an intravenous infusion over about 60 minutes twice every three weeks. MEDIO680 can be administered as an intravenous infusion over about 60 minutes once every three weeks. Administration of MEDIO680 can be continuous. Administration of MEDIO680 can be intermittent.

In some embodiments, SHR-1210 can be administered at an amount of about 0.5 to about 20 mg/kg (including, for example, 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg, 20 mg/kg), QW. In some embodiments, SHR-1210 can be administered at an amount of about 0.5 to about 20 mg/kg (including, for example, 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg, 20 mg/kg), Q2W. In some embodiments, SHR-1210 can be administered at an amount of about 0.5 to about 20 mg/kg (including, for example, 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg, 20 mg/kg), Q3W. In some embodiments, SHR-1210 can be administered at an amount of about 0.5 to about 20 mg/kg (including, for example, 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg, 20 mg/kg), Q4W. Administration of SHR-1210 can be continuous. Administration of SHR-1210 can be intermittent.

SHR-1210 can be administered as an intravenous infusion over about 10, 20, 30, 40, 50, or 60 or more minutes. SHR-1210 can be administered as an intravenous infusion over about 60 minutes once every 1, 2, 3, 4, 5 or more weeks. SHR-1210 can be administered as an intravenous infusion over about 60 minutes once every two weeks. SHR-1210 can be administered as an intravenous infusion over about 60 minutes twice every three weeks. SHR-1210 can be administered as an intravenous infusion over about 60 minutes once every three weeks. Administration of SHR-1210 can be continuous. Administration of SHR-1210 can be intermittent.

The combinations described herein can be administered in a regimen. The regimen can be structured to provide therapeutically effective amounts of a compound of Formula I, or a pharmaceutically acceptable salt thereof, and a PD-1 inhibitor (e.g., an anti-PD-1 antibody) over a predetermined period of time (e.g., an administration time). The regimen can be structured to limit or prevent side-effects or undesired complications of each of the components of the combination described herein. The regimen can be structured in a manner that results in increased effect for both therapies of the combination (e.g., synergy). Regimens useful for treating cancer can include any number of days of administration which can be repeated as necessary. Administration periods can be broken by a rest period that includes no administration of at least one therapy. In some embodiments, a regimen can include administration periods that include 2, 3, 5, 7, 10, 15, 21, 28, or more days. These periods can be repeated. In some embodiments, a regimen can include a set number of days as previously described where the regimen is repeated 1, 2, 3, 4, 5, 6, 7, 8, 9, 10, 11, 12, 13, or more times.

Regimens can include a rest period of at least 1, 2, 3, 5, 7, 10, or more days, where at least one therapy is no longer administered to a patient. The rest period can be determined by, for example, monitoring the reaction of the patient to the drug or by measuring the efficacy of the treatment. A rest period can be applicable to a single therapy, such that only one therapy of a combination described herein is discontinued in the rest period but the other therapy(ies) are still administered. Rest periods can be applied to all of the therapies administered to the subject such that the subject receives no therapy for a set period of time during the rest period.

Regimens described herein for the treatment of cancer using the combinations described herein can be continued until disease progression or unacceptable toxicity.

Regimens for administration of combinations described herein include, for example, administration of a compound of Formula I, or a pharmaceutically acceptable salt thereof, BIW or TIW and administration of a PD-1 inhibitor. For example, a compound of Formula I, or a pharmaceutically acceptable salt thereof, can be administered QD for about 21 days and an anti-PD-1 antibody described herein can be administered Q2W or Q4W). For example, a compound of Formula I, or a pharmaceutically acceptable salt thereof, can be administered BIW or TIW and an anti-PD-1 antibody described herein can be administered Q2W. In another exemplary regimen, a compound of Formula I, or a pharmaceutically acceptable salt thereof, can be administered BIW or TIW and an anti-PD-1 antibody can be administered BIW for 2 or 3 weeks. In some exemplary regimens, a compound of Formula I, or a pharmaceutically acceptable salt thereof, can be administered BIW or TIW and an anti-PD-1 antibody can be administered Q3W. In some exemplary regimens, a compound of Formula I, or a pharmaceutically acceptable salt thereof, can be administered BIW and a PD-1 inhibitor described herein can be administered QW, Q2W, or Q3W. In some instances, such regimens include administration of PD-1 antibody administered QW, Q2W, or Q3W. In some exemplary regimens, a compound of Formula I, or a pharmaceutically acceptable salt thereof, can be administered TIW and a PD-1 inhibitor described herein can be administered QW, Q2W, or Q3W. In some instances, such regimens include administration of PD-1 antibody administered QW, Q2W, or Q3W. In some instances, such regimens include administration of a compound of Formula I, or a pharmaceutically acceptable salt thereof, administered QD. In some instances, such regimens include administration of a compound of Formula I, or a pharmaceutically acceptable salt thereof, administered QD for at least 21 days. In some exemplary regimens, a compound of Formula I, or a pharmaceutically acceptable salt thereof, can be administered QD or QW and a PD-1 inhibitor (e.g., an anti-PD-1 antibody) is administered QW, Q2W, or Q3W.

The regimen can be a regimen for administration of pembrolizumab with a compound of Formula I, or a pharmaceutically acceptable salt thereof, as described herein. In some exemplary regimens including pembrolizumab, a compound of Formula I, or a pharmaceutically acceptable salt thereof, can be administered BIW or TIW and pembrolizumab is administered in accordance with the prescribing information provided in, for example, a package insert. In another exemplary regimen, pembrolizumab is administered at an amount of about 1 mg/kg to about 10 mg/kg on day 1 of the regimen, and BIW for at least three weeks thereafter until disease progression or unacceptable toxicity and a compound of Formula I, or a pharmaceutically acceptable salt thereof, is administered BIW or TIW over the same period of time. In another exemplary regimen, pembrolizumab is administered at an amount of about 1 mg/kg to about 10 mg/kg on day 1 of a regimen, and once Q3W thereafter until disease progression or unacceptable toxicity and a compound of Formula I, or a pharmaceutically acceptable salt thereof, is administered BIW or TIW over the same period of time. Pembrolizumab can be administered BIW for 3 weeks with a compound of Formula I, or a pharmaceutically acceptable salt thereof, where the compound of Formula I, or a pharmaceutically acceptable salt thereof, is administered, for example, BIW or TIW during the course of such a regimen. Pembrolizumab can be administered QW for 3 weeks with a compound of Formula I, or a pharmaceutically acceptable salt thereof, where the compound of Formula I, or a pharmaceutically acceptable salt thereof, is administered, for example, BIW or TIW during the course of such a regimen. In some exemplary regimens, pembrolizumab can be administered QW for 3 weeks with a compound of Formula I, or a pharmaceutically acceptable salt thereof, where the compound of Formula I, or a pharmaceutically acceptable salt thereof, is administered, for example, QD or QW during the course of such a regimen. Such regimens can be repeated as described above (e.g., 1, 2, 3, 4, 5, 6, 7, 8, 9, 10, 11, 12, or more times).

In another exemplary regimen including pembrolizumab, a compound of Formula I, or a pharmaceutically acceptable salt thereof, can be administered QD and pembrolizumab is administered in accordance with the prescribing information provided in, for example, a package insert. In another exemplary regimen, pembrolizumab is administered at an amount of about 1 mg/kg to about 10 mg/kg on day 1 of the regimen, and BIW for at least three weeks thereafter until disease progression or unacceptable toxicity and a compound of Formula I, or a pharmaceutically acceptable salt thereof, is administered QD over the same period of time. In another exemplary regimen, pembrolizumab is administered at an amount of about 1 mg/kg to about 10 mg/kg on day 1 of a regimen, and once Q3W thereafter until disease progression or unacceptable toxicity and a compound of Formula I is administered QD over the same period of time. Pembrolizumab can be administered BIW for 3 weeks with a compound of Formula I, or a pharmaceutically acceptable salt thereof, where the compound of Formula I, or a pharmaceutically acceptable salt thereof, is administered, for example, QD during the course of such a regimen. Pembrolizumab can be administered QW for 3 weeks with a compound of Formula I, where the compound of Formula I is administered, or a pharmaceutically acceptable salt thereof, for example, QD during the course of such a regimen. Such regimens can be repeated as described above (e.g., 1, 2, 3, 4, 5, 6, 7, 8, 9, 10, 11, 12, or more times).

The regimen can be a regimen for administration of nivolumab with a compound of Formula I, or a pharmaceutically acceptable salt thereof, as described herein. In some exemplary regimens including nivolumab, a compound of Formula I, or a pharmaceutically acceptable salt thereof, can be administered BIW or TIW and nivolumab is administered in accordance with the prescribing information provided in, for example, a package insert. In another exemplary regimen, nivolumab is administered at an amount of about 1 mg/kg to about 5 mg/kg on day 1 and BIW for 3 weeks thereafter until disease progression or unacceptable toxicity and a compound of Formula I, or a pharmaceutically acceptable salt thereof, is administered BIW or TIW over the same period of time. In some exemplary regimens, nivolumab is administered at an amount of about 1 mg/kg to about 5 mg/kg on day 1 and Q2W thereafter until disease progression or unacceptable toxicity and a compound of Formula I is administered BIW or TIW over the same period of time. In some exemplary regimens, nivolumab can be administered Q2W, where the compound of Formula I, or a pharmaceutically acceptable salt thereof, is administered, for example, BIW or TIW during the course of such a regimen. In some exemplary regimens, nivolumab can be administered Q2W, where the compound of Formula I, or a pharmaceutically acceptable salt thereof, is administered, for example, QD or QW during the course of such a regimen. Such regimens can be repeated as described above (e.g., 1, 2, 3, 4, 5, 6, 7, 8, 9, 10, 11, 12, or more times).

In another exemplary regimen including nivolumab, a compound of Formula I, or a pharmaceutically acceptable salt thereof, can be administered QD and nivolumab is administered in accordance with the prescribing information provided in, for example, a package insert. In another exemplary regimen, nivolumab is administered at an amount of about 1 mg/kg to about 5 mg/kg on day 1 and BIW for 3 weeks thereafter until disease progression or unacceptable toxicity and a compound of Formula I, or a pharmaceutically acceptable salt thereof, is administered QD over the same period of time. In some exemplary regimens, nivolumab is administered at an amount of about 1 mg/kg to about 5 mg/kg on day 1 and Q2W thereafter until disease progression or unacceptable toxicity and a compound of Formula I, or a pharmaceutically acceptable salt thereof, is administered QD over the same period of time. In some exemplary regimens, nivolumab can be administered Q2W, where the compound of Formula I, or a pharmaceutically acceptable salt thereof, is administered, for example, QD during the course of such a regimen. Such regimens can be repeated as described above (e.g., 1, 2, 3, 4, 5, 6, 7, 8, 9, 10, 11, 12, or more times).

It should also be appreciated that the combinations described herein for treating cancer can be coadministered with other active agents other than those present in the combinations described herein (e.g., anti-cancer agents). Regimens for administration of a combination described herein, including the exemplary regimens set forth above, can be modified as necessary to include administration of such active agents. Administration of such active agents, e.g., anti-cancer agents, can be performed QD, QW, QM, BID, BIW, TIW, Q2W, Q3W, or Q4W, or in accordance with prescribing information for such anti-cancer agents as set forth, for example, in a package insert. Exemplary anti-cancer agents include but are not limited to: ABRAXANE; abiraterone; ace-11; aclarubicin; acivicin; acodazole hydrochloride; acronine; actinomycin; acylfulvene; adecypenol; adozelesin; adriamycin; aldesleukin; all trans-retinoic acid (ATRA); altretamine; ambamustine; ambomycin; ametantrone acetate; amidox; amifostine; aminoglutethimide; aminolevulinic acid; amrubicin; amsacrine; anagrelide; anastrozole; andrographolide; antarelix; anthramycin; aphidicolin glycinate; apurinic acid; ara-CDP-DL PTBA; arginine deaminase; ARRY-162; ARRY-300; ARRY-142266; AS703026; asparaginase; asperlin; asulacrine; atamestane; atrimustine; axinastatin 1; axinastatin 2; axinastatin 3; azasetron; azatoxin; azatyrosine; azacitidine; AZD8330; azetepa; azotomycin; balanol; batimastat; BAY 11-7082; BAY 43-9006; BAY 869766; bendamustine; benzochlorins; benzodepa; benzoylstaurosporine; beta-alethine; betaclamycin B; betulinic acid; b-FGF inhibitor; bicalutamide; bisantrene; bisaziridinylspermine; bisnafide; bisnafide dimesylate; bistratene A; bisantrene hydrochloride; bleomycin; bleomycin sulfate; busulfan; bizelesin; breflate; bortezomib; brequinar sodium; bropirimine; budotitane; buthionine sulfoximine; bryostatin; cactinomycin; calusterone; calcipotriol; calphostin C; camptothecin derivatives; capecitabine; carboxamide-amino-triazole; carboxyamidotriazole; CaRest M3; CARN 700; caracemide; carbetimer; carboplatin; carmustine; carubicin hydrochloride; carzelesin; castanospermine; cecropin B; cedefingol; celecoxib; cetrorelix; chlorins; chloroquinoxaline sulfonamide; cicaprost; chlorambucil; Chlorofusin; cirolemycin; cisplatin; CI-1040; cis-porphyrin; cladribine; clomifene analogues; clotrimazole; collismycin A; collismycin B; combretastatin A4; combretastatin analogue; conagenin; crambescidin 816; crisnatol; crisnatol mesylate; cryptophycin 8; cryptophycin A derivatives; curacin A; cyclopentanthraquinones; cycloplatam; cypemycin; cyclophosphamide; cytarabine; cytarabine ocfosfate; cytolytic factor; cytostatin; dacarbazine; dactinomycin; daunorubicin; daunorubicin hydrochloride; decarbazine; dacliximab; dasatinib; decitabine; dehydrodidemnin B; deslorelin; dexamethasone; dexifosfamide; dexrazoxane; dexverapamil; dexormaplatin; dezaguanine; dezaguanine mesylate; diaziquone; didemnin B; didox; diethylnorspermine; dihydro 5 azacytidine; dihydrotaxol; 9-dioxamycin; diphenyl spiromustine; docosanol; dolasetron; docetaxel; doxorubicin; doxorubicin hydrochloride; doxifluridine; droloxifene; droloxifene citrate; dromostanolone propionate; dronabinol; duazomycin; duocarmycin SA; ebselen; ecomustine; edelfosine; edrecolomab; edatrexate; eflornithine hydrochloride; eflornithine; elemene; emitefur; elsamitrucin; enloplatin; enpromate; epipropidine; epirubicin; epirubicin hydrochloride; epristeride; erbulozole; eribulin; esorubicin hydrochloride; estramustine; estramustine phosphate sodium; etanidazole; etoposide; etoposide phosphate; etoprine; exemestane; fadrozole; fadrozole hydrochloride; fazarabine; fenretinide; filgrastim; finasteride; flavopiridol; flezelastine; fluasterone; floxuridine; fludarabine phosphate; fludarabine; fluorodaunorubicin hydrochloride; forfenimex; formestane; fluorouracil; floxouridine; flurocitabine; fosquidone; fostriecin sodium; fostriecin; fotemustine; gadolinium texaphyrin; gallium nitrate; galocitabine; ganirelix; gelatinase inhibitors; gemcitabine; geldanamycin; gossyphol; GDC-0973; GSKI 120212/trametinib; herceptin; hydroxyurea; hepsulfam; heregulin; hexamethylene bisacetamide; hypericin; ibandronic acid; ibrutinib; idarubicin; idarubicin hydrochloride; ifosfarnide; canfosfarnide; ilmofosine; iproplatin; idoxifene; idramantone; ilmofosine; ilomastat; imidazoacridones; imatinib (e.g., GLEEVEC); imiquimod; iobenguane; iododoxorubicin; ipomeanol; irinotecan; irinotecan hydrochloride; irsogladine; isobengazole; isohomohalicondrin B; itasetron; iimofosine; interleukin IL-2 (including recombinant interleukin II; or rlL.sub.2); interferon alfa-2a; interferon alfa-2b; interferon alfa-nl; interferon alfa-n3; interferon beta-1a; interferon gamma-1b; jasplakinolide; kahalalide F; lamellarin N triacetate; lanreotide; leinamycin; lenograstim; lentinan sulfate; leptolstatin; letrozole; leuprorelin; levarnisole; lenalidomide; lenvatinib; liarozole; lissoclinamide 7; lobaplatin; lombricine; lometrexol; lonidarnine; losoxantrone; lovastatin; loxoribine; lurtotecan; lutetium texaphyrin; lysofylline; lanreotide acetate; lapatinib; letrozole; leucovorin; leuprolide acetate; liarozole hydrochloride; lometrexol sodium; lomustine; losoxantrone hydrochloride; pomalidomide; LY294002; maitansine; mannostatin A; marimastat; masoprocol; maspin; matrilysin inhibitors; menogaril; merbarone; meterelin; methioninase; metocloprarnide; MIF inhibitor; mifepristone; miltefosine; mirimostim; mitoguazone; mitolactol; mitonafide; mitoxantrone; mofarotene; molgramostim; mopidamol; mycaperoxide B; myriaporone; maytansine; mechlorethamine hydrochloride; megestrol acetate; melengestrol acetate; melphalan; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nafarelin; nagrestip; napavin; naphterpin; nartograstim; nedaplatin; nemorubicin; neridronic acid; nilutamide; nisamycin; nitric oxide modulators; nitroxide antioxidant; nitrullyn; nocodazole; nogalamycin; oblimersen (GENASENSE); octreotide; okicenone; oligonucleotides; onapristone; ondansetron; ondansetron; oracin; oral cytokine inducer; ormaplatin; oxisuran; oxaloplatin; osaterone; oxaliplatin; oxaunomycin; palauarnine; palmitoylrhizoxin; parnidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; porfiromycin; prednisone; prostaglandin J2; pyrazoloacridine; paclitaxel; PD035901; PD184352; PD318026; PD98059; peliomycin; pentamustine; peplomycin sulfate; PKC412; pipobroman; piposulfan; piroxantrone hydrochloride; plicamycin; plomestane; podophyllotoxin; polyphenol E; porfi.mer sodium; porfiromycin; prednimustine; procarbazine; procarbazine hydrochloride; puromycin; puromycin hydrochloride; pyrazofurin; raltitrexed; ramosetron; retelliptine demethylated; rhizoxin; rituximab; RII retinamide; rogletimide; rohitukine; romurtide; roquinimex; rubiginone B1; ruboxyl; riboprine; romidepsin; safingol; safingol hydrochloride; saintopin; sarcophytol A; sargramostim; semustine; sizofiran; sobuzoxane; sodium borocaptate; sodium phenylacetate; solverol; sonermin; sorafenib; sunitinib; sparfosic acid; spicamycin D; spiromustine; splenopentin; spongistatin 1; Spongistatin 2; Spongistatin 3; Spongistatin 4; Spongistatin 5; Spongistatin 6; Spongistatin 7; Spongistatin 8; and Spongistatin 9; squalamine; stipiamide; stromelysin inhibitors; sulfinosine; suradista; suramin; swainsonine; SB239063; selumetinib/AZD6244; simtrazene; SP600125; sparfosate sodium; sparsomycin; spirogermanium hydrochloride; spiroplatin; streptonigrin; streptozocin; sulofenur; tallimustine; tamoxifen methiodide; tauromustine; tazarotene; tecogalan sodium; tegafur; tellurapyrylium; temoporfin; temozolomide; teniposide; tetrachlorodecaoxide; tetrazomine; thaliblastine; thiocoraline; thrombopoietin; thymalfasin; thymopoietin receptor agonist; thymotrinan; tirapazamine; titanocene bichloride; topsentin; toremifene; tretinoin; triacetyluridine; triciribine; trimetrexate; triptorelin; tropisetron; turosteride; tyrphostins; talisomycin; TAK-733; taxotere; tegafur; teloxantrone hydrochloride; teroxirone; testolactone; thiamiprine; thioguanine; thiotepa; tiazofurin; tirapazamine; toremifene citrate; trastuzumab; trestolone acetate; triciribine phosphate; trimetrexate; trimetrexate glucuronate; triptorelin; tubulozole hydrochloride; tumor necrosis factor-related apoptosis-inducing ligand (TRAIL); UBC inhibitors; ubenimex; U0126; uracil mustard; uredepa; vapreotide; variolin B; velaresol; veramine; verteporfin; vinorelbine; vinxaltine; vitaxin; vinblastine; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; wortmannin; XL518; zanoterone; zeniplatin; zilascorb; zinostatin stimalamer; zinostatin; and zorubicin hydrochloride.

Other exemplary anti-cancer agents include Erbulozole (e.g., R-55104); Dolastatin 10 (e.g., DLS-10 and NSC-376128); Mivobulin isethionate (e.g., CI-980); NSC-639829; Discodermolide (e.g., NVP-XX-A-296); ABT-751 (Abbott; e.g., E-7010); Altorhyrtin A; Altorhyrtin C; Cemadotin hydrochloride (e.g., LU-103793 and NSC-D-669356); Epothilone A; Epothilone B; Epothilone C; Epothilone D; Epothilone E; Epothilone F; Epothilone B N-oxide; Epothilone AN-oxide; 16-aza-epothilone B; 21-aminoepothilone B; 21-hydroxyepothilone D; 26-fluoroepothilone; Auristatin PE (e.g., NSC-654663); Soblidotin (e.g., TZT-1027); LS-4559-P (Pharmacia; e.g., LS-4577); LS-4578 (Pharmacia; e.g., LS-477-P); LS-4477 (Pharmacia); LS-4559 (Pharmacia); RPR-112378 (Aventis); DZ-3358 (Daiichi); FR-182877 (Fujisawa; e.g., WS-9265B); GS-164 (Takeda); GS-198 (Takeda); KAR-2 (Hungarian Academy of Sciences); BSF-223651 (BASF; e.g., ILX-651 and LU-223651); SAH-49960 (Lilly/Novartis); SDZ-268970 (Lilly/Novartis); AM-97 (Armad/Kyowa Hakko); AM-132 (Armad); AM-138 (Armad/Kyowa Hakko); IDN-5005 (Indena); Cryptophycin 52 (e.g., LY-355703); AC-7739 (Ajinomoto; e.g., AVE-8063A and C⁵-39.HCl); AC-7700 (Ajinomoto; e.g., AVE-8062; AVE-8062A; CS-39-L-Ser.HCl; and RPR-258062A); Vitilevuamide; Tubulysin A; Canadensol; CA-170 (Curis, Inc.); Centaureidin (e.g., NSC-106969); T-138067 (Tularik; e.g., T-67; TL-138067 and TI-138067); COBRA-1 (Parker Hughes Institute; e.g., DDE-261 and WHI-261); H10 (Kansas State University); H16 (Kansas State University); Oncocidin Al (e.g., BTO-956 and DIME); DDE-313 (Parker Hughes Institute); Fijianolide B; Laulimalide; SPA-2 (Parker Hughes Institute); SPA-1 (Parker Hughes Institute; e.g., SPIKET-P); 3-IAABU (Cytoskeleton/Mt. Sinai School of Medicine; e.g., MF-569); Narcosine (e.g., NSC-5366); Nascapine; D-24851 (Asta Medica); A-105972 (Abbott); Hemiasterlin; 3-BAABU (Cytoskeleton/Mt. Sinai School of Medicine; e.g., MF-191); TMPN (Arizona State University); Vanadocene acetylacetonate; T-138026 (Tularik); Monsatrol; lnanocine (e.g., NSC-698666); 3-IAABE (Cytoskeleton/Mt. Sinai School of Medicine); A-204197 (Abbott); T-607 (Tuiarik; e.g., T-900607); RPR-115781 (Aventis); Eleutherobins (e.g., Desmethyleleutherobin; Desaetyleleutherobin; 1soeleutherobin A; and Z Eleutherobin); Caribaeoside; Caribaeolin; Halichondrin B; D-64131 (Asta Medica); D-68144 (Asta Medica); Diazonamide A; A-293620 (Abbott); NPI-2350 (Nereus); Taccalonolide A;TUB-245 (Aventis); A-259754 (Abbott); Diozostatin; (-)-Phenylahistin (e.g., NSCL-96F037); D-62638 (Asta Medica); D-62636 (Asta Medica); Myoseverin B; D-43411 (Zentaris; e.g., D-81862); A-289099 (Abbott); A-318315 (Abbott); HTI-286 (e.g., SPA-110; trifluoroacetate salt) (Wyeth); D-82317 (Zentaris); D-82318 (Zentaris); SC-12983 (NCI); Resverastatin phosphate sodium; BPR-OY-007 (National Health Research Institutes); and SSR-250411 (Sanofi)); goserelin; leuprolide; triptolide; homoharringtonine; topotecan; itraconazole; deoxyadenosine; sertraline; pitavastatin; clofazimine; 5-nonyloxytryptamine; vemurafenib; dabrafenib; gefitinib (IRESSA); erlotinib (TARCEVA); cetuximab (ERBITUX); lapatinib (TYKERB); panitumumab (VECTIBIX); vandetanib (CAPRELSA); afatinib/BIBW2992; CI-1033/canertinib; neratinib/HKI-272; CP-724714; TAK-285; AST-1306; ARRY334543; ARRY-380; AG-1478; dacomitinib/PF299804; OSI-420/desmethyl erlotinib; AZD8931; AEE726; pelitinib/EKB-569; CUDC-101; WZ8040; WZ4002; WZ3146; AG-490; XL647; PD153035; 5-azathioprine; 5-aza-2′-deoxycytidine; 17-N-Allylamino-17-Demethoxygeldanamycin (17-AAG); 20-epi-1,25 dihydroxyvitamin D3; 5 ethynyluracil; and BMS-599626.

In certain embodiments, the combinations described herein are coadministered with an anti-cancer agent described above, where the anti-cancer agent has known activity against a particular cancer (e.g., gemcitibine coadministered with a combination described herein for treating pancreatic cancer). The anti-cancer agents above can be approved for use in treating certain indications (e.g., certain cancers) at concentrations, amounts, and using treatment regimens known in the art.

It is understood that modifications which do not substantially affect the activity of the various embodiments of the invention described herein are also included within the scope of the invention provided herein. Accordingly, the following examples are intended to illustrate but not limit the scope of the invention.

### EXAMPLES

### Example 1: In Vivo Efficacy of Combination Treatment with HBI-2375 and Anti-PD-1 Antibody in Syngeneic Xenograft Model Tumors

Reagents: The WDR5 inhibitor (HBI-2375, Formula (Id)) was supplied by Wuxi AppTec. Anti-PD-1 (RMP1-14) antibodies were purchased from BioXCell (China).

Cell lines and maintenance: Mouse colon cancer cell line, MC38, Mouse lung cancer cell line, 3LL, or mouse breast cancer cell line, E0771, was supplied as part of Wuxi AppTec cell bank and were cultured in media according to source at 37° C. and 5% C₀₂.

Animal models and in vivo treatments: Procedures involving the care and use of animals in this study were reviewed and approved by the Institutional Animal Care and Use Committee (IACUC) of Wuxi AppTec prior to execution. During the study, the care and use of animals were conducted in accordance with the regulations of the Association for Assessment and Accreditation of Laboratory Animal Care (AAALAC). Animals (6-8 weeks) were obtained from Beijing VR Laboratory Animal Co., LTD and were allowed to acclimate prior to tumor cell inoculation.

All cell lines were maintained in culture and cells in exponential growth phase were harvested and quantitated by cell counter before tumor inoculation. MC38 tumor cells (1×10⁶) in 0.1 ml of PBS, 3LL tumor cells (2×10⁶) in 0.1 ml of PBS or E0771 tumor cells (5×10⁶), were inoculated onto the right front flank of female C₅₇/B1 animals, mean tumor size approximately 115 mm³, 63 mm³ and 67 mm³ respectively at the start of treatment.

The date of randomization and treatment initiation was denoted as day 0. Tumor volumes were measured twice per week in two dimensions using a caliper, and the volume will be expressed in mm³ using the formula: V=(L×W×W)/2, where V is tumor volume, L is tumor length (the longest tumor dimension) and W is tumor width (the longest tumor dimension perpendicular to L).

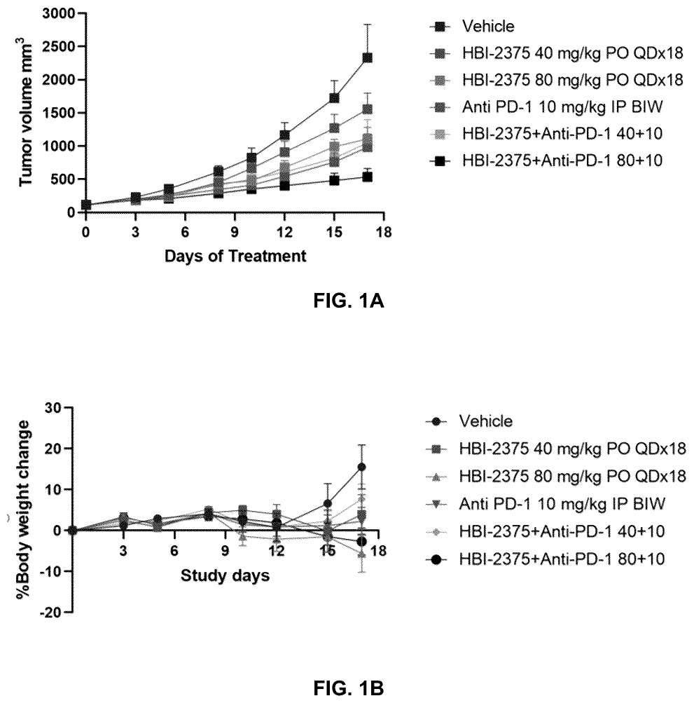

Dosing volume was 10 mL/kg/day. HBI-2375 was dissolved 10% DMSO/10% solutol HS/80% Water and dosed at either 40 mg/kg QD or 80 mg/kg QD orally. Anti-PD-1 was diluted in PBS and dosed at 10 mg/kg by intraperitoneal injection twice a week. The results of this study are depicted in FIGS. 1A-B (MC38), 2A-B (E0771), and 3A-B (3LL).

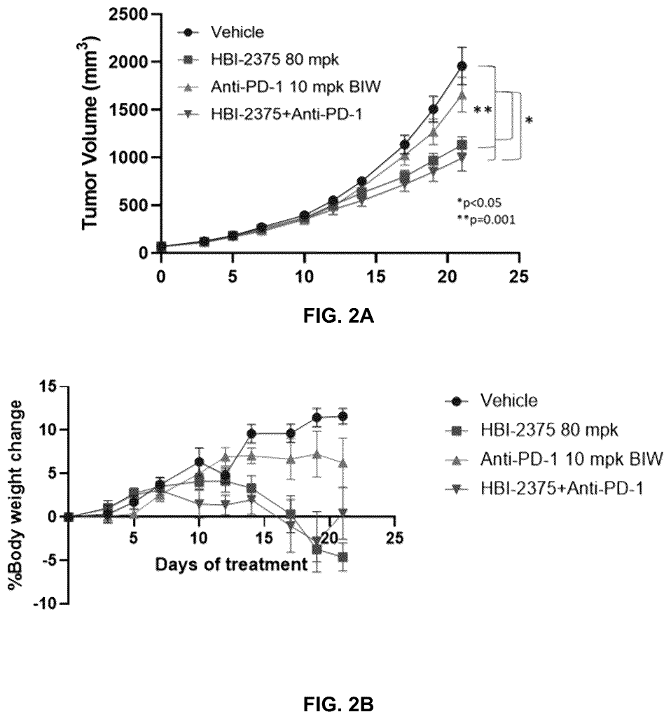

The results show that treatment with a combination of the compound of HBI-2375 and anti PD-1 resulted in significant and sustained reduction in tumor volume (see FIGS. 1A, 2A and 3A) compared to the tumor reduction observed with varying dosages of anti-PD-1 and HBI-2375 alone. Similar effect was observed on percent body weight change for all study groups. See FIGS. 1B, 2B and 2C. Collectively, these results evidence a synergistic effect between the anti-PD-1 and HBI-2375.

### FACS Analysis

Upon termination, tumors and blood were collected at 12 hours post last dose on dosing day 17 for MC38, day 14 for 3LL and on day 21 for E0771. Necrotic or ulcerated tumor tissue was removed, and the tumor was rinsed in PBS. A portion of the tumor was collected in RNAlater for RNA analysis. Some portion was sperately minced and snap frozen immediately for protein isolation. Another portion of the tumor was fixed in 10% neutral buffered formalin prior to processing into paraffin blocks and the rest of the tumor dissociated for FACs analysis.

Tumor and blood immunotyping: Animals bearing MC38, 3LL and E0771 tumors were terminated 12 hours post last dose. These animals were further harvested for tumors and blood accordingly. Tumors were enzymatically and mechanically dissociated using The Tumor Dissociation Kit (130-096-730) Miltenyi Biotec MACS Technology and processed for FACs analysis according to manufacturer's instructions. To obtain single cells suspension from whole blood, anticoagulant blood samples were lysed by 1× Red Blood Cell Lysis Solution with 1:19 dilution factor twice. After lysis at room temperature, cells were washed and counted.

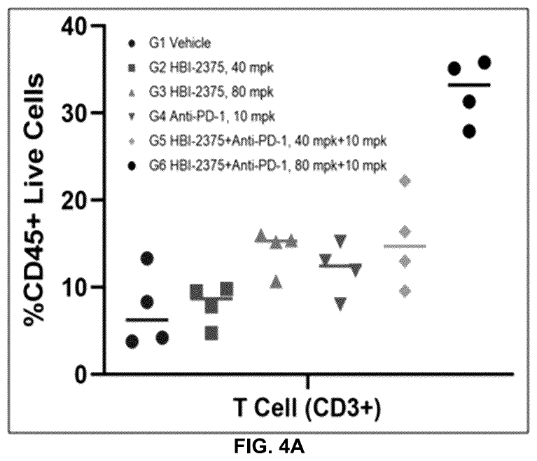

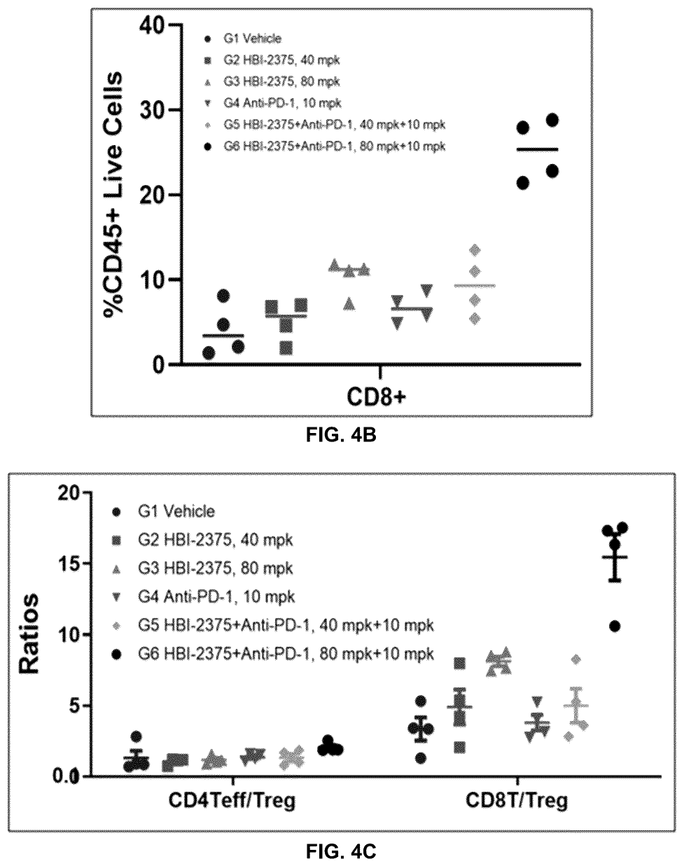

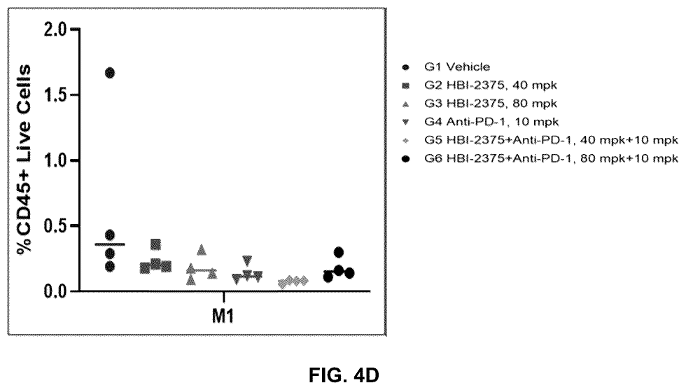

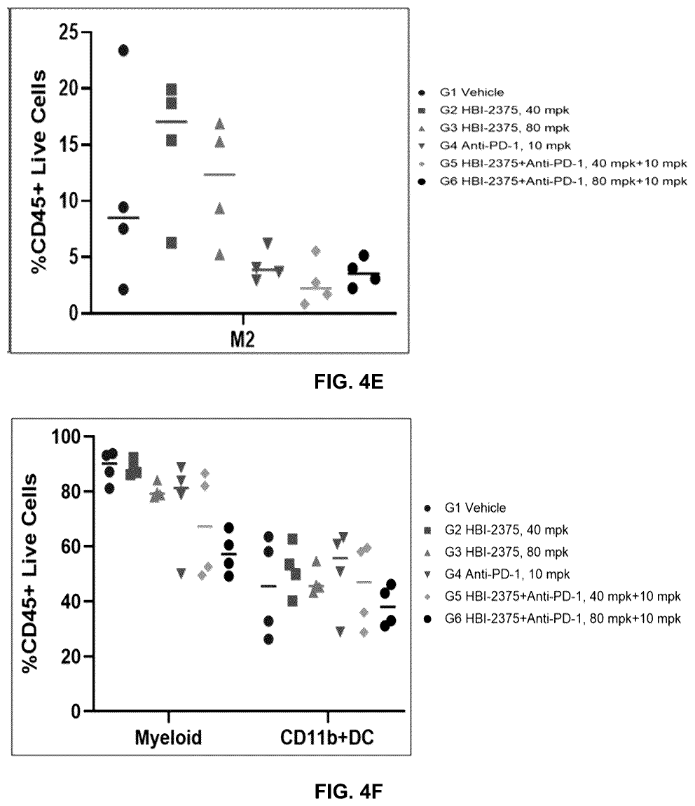

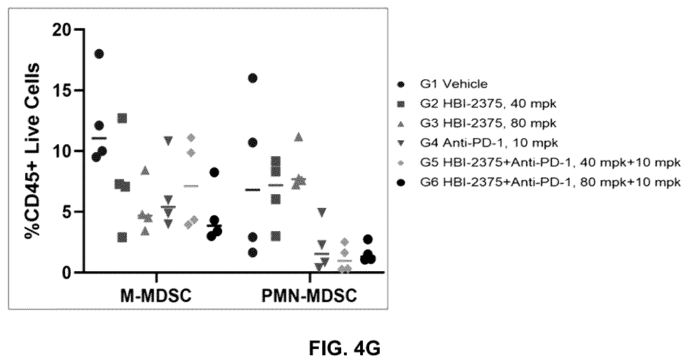

Tumor cell suspension and whole lysed blood were resuspended and blocked in staining buffer with 1 μg/ml Fc-Block (Mouse BD Fc Block™ CAT #553142). All antibodies were diluted in 1x DPBS buffer except CD206 and FOXP3 which were diluted in 1x Permeabilization Buffer. Primary and Secondary Antibodies dilutions and incubation times were according to Wuxi AppTec optimizations. Live/Dead cell dye, and antibodies for CD45, CD3, CD25, CD335, CD19, Ly6C, CD206, CD11c were purchased from Biolegend. Ly6G, F4/80 and MHCII was purchased from BD Biosciences. CD4, CD8, FoxP3, and CD11b were purchased from eBiosciences. Data was collected on a cytometer and data was analyzed using FlowJo software. Graphpad Prism, SPSS or Excel were used for statistical analysis. The results of this study are depicted in FIGS. 4A-4G for MC38 model.

It is known in the art that utilization of a syngenic mouse model like MC38, 3LL or E0771 allows for a complete immune landscape of the tumor microenvironment, which is key for adaptive immunotherapy studies. Such mouse models must satisfy the following criteria: (1) responsiveness to known immune-modulating therapies (e.g., anti-PD-1); (2) extent and composition of tumor-infiltrating leukocytes (TIL); and (3) immunogenicity. MC38, 3LL and E0771 satisfy these criteria. Thus, such models have been routinely used by those skilled in the art to test compounds that affect an immune response within the tumor microenvironment. The main characteristics of a MC38 mouse model, for example, is its ability to recruit immune infiltrating cells. Therefore, the in vivo MC38 model allows studying the role of immune cells in tumor growth and response to therapies that affect tumor growth via activity of tumor immunity. However, this does not necessarily mean that the main clinical indication should be the same as the preclinical indication (in case of MC38 model-colon cancer). For example, Nivolumab (an approved anti-PD1 mAb) was initially tested in the MC38 model and was later approved by FDA in melanoma, NSCLC, RCC, Hodgkin lymphoma, squamous cell carcinoma, hepatocellular carcinoma, and colorectal cancer. Like MC38, 3LL and E0771 are art-recognized syngeneic tumor models, which permit one skilled in the art to interrogate the effect of putative active agents on the tumor microenvironment. Thus, in vivo testing with the MC38, 3LL and/or E0771 allows for evaluation of likely efficacy in humans for treatment of a broad range of immunoresponsive tumor types, including but not limited to melanoma, NSCLC, RCC and other immunoresponsive tumor types.

### Example 2: CD8+ T Cell Infiltration and Differential Gene Expression in MC38 and 3LL Xenografts

Immunohistochemistry and Immunofluorescence: Freshly collected tumor tissues from Example 1 (MC38 and 3LL, N=3 each) were placed in 10% NBF and fixed for 24 hours at RT. Tumor tissue was trimmed and rinsed in running water. The specimens were transferred to the Vacuum Tissue Processor (VP1-JC, SAKURA) for dehydration, then embedded into FFPE blocks using Tissue embedding center (TEC 5-EM JC-2, SAKURA). FFPE blocks were sectioned with a manual rotary microtome (HistoCore MULTICUT, Leica), 4 μm thickness/section. Reagents for IHC and antigen retrieval including Bond™ Epitope Retrieval Solution 1 (Bond ER1), Bond™ Epitope Retrieval Solution 2 (Bond ER2), Bond™ Dewax Solution, Bond™ Wash Solution 10× concentrate, Bond™ Polymer Refine Detection, and Primary Antibody Diluent (ready-to-use) were purchased from Leica. CD antibodies (CST #98941) were purchased from Cell Signaling.

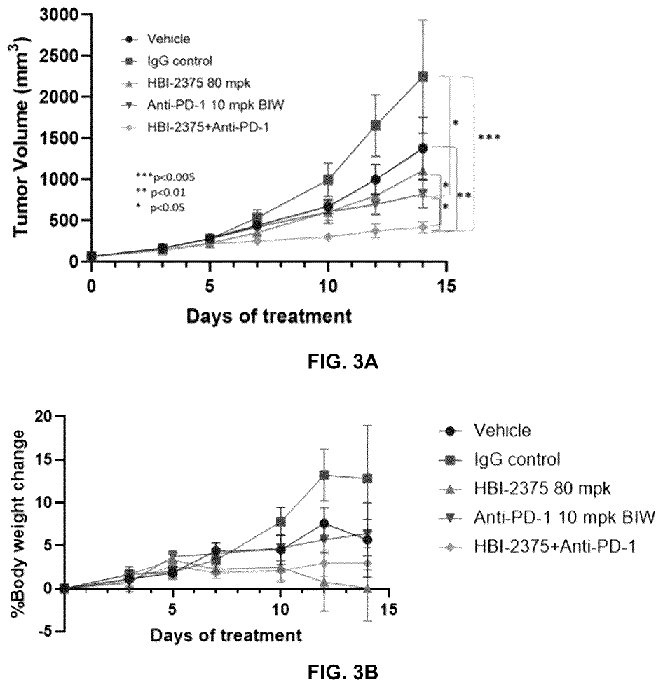

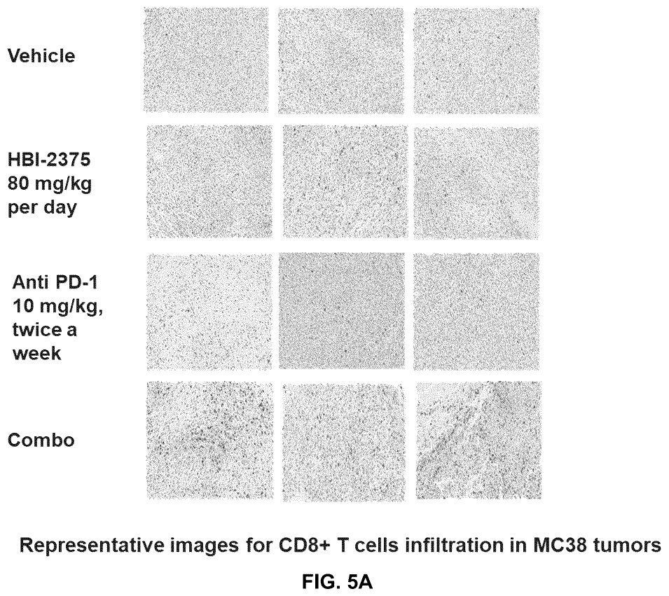

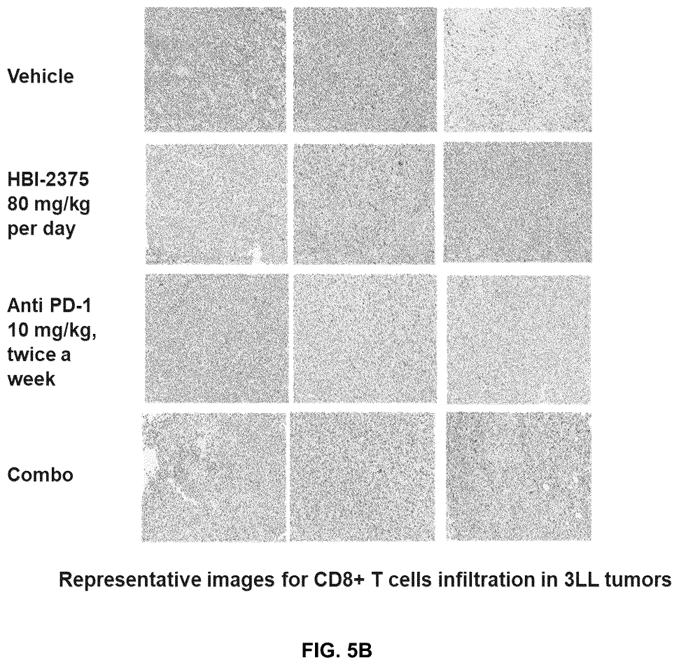

Formalin-fixed, paraffin-embedded tissue from harvested tumors was immunohistochemically stained with a single antibody for CD8 and counterstained with H&E. All stained sections were scanned with Pannoramic Digital Slide Scanners (3DHISTECH, Pannoram ic SCAN). High resolution picture for whole section was generated and further analyzed. All the images were analyzed with HALO™ platform. The whole slide image was analyzed and regions of necrosis were excluded. For CD8, the ratio of the positive cell counts against the area of whole tumor was scored. Positive cell density=positive cell counts/whole tumor area ×100%. The results of the MC38 tumor study is depicted in FIG. 5A and the results of the 3LL tumor study is depicted FIG. 5B. As can be seen in the digital slides, the combination of HBI-2375 (80 mg/kg/day) and anti-PD-1 antibody (10 mg/kg, BIW), greatly increased CD8+ T cell infiltration into the MC38 (FIG. 5A) and 3LL (FIG. 5B) xenografts.

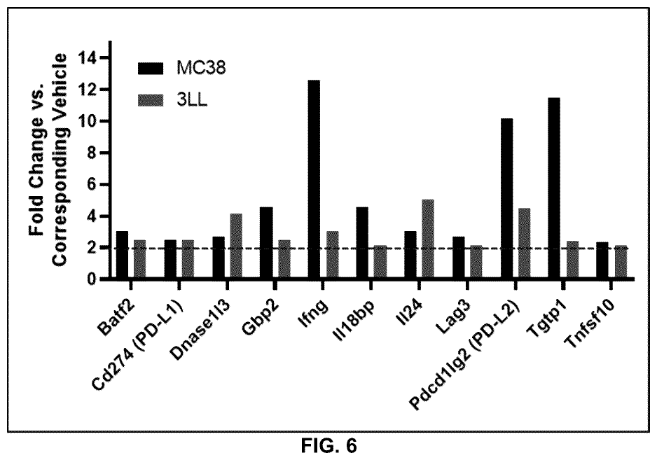

RNA bioinformatics: RNA was extracted from the tumor sections from Example 1 (MC38 and 3LL) and NanoDrop was used to determine the concentration of RNA. The mRNAs containing oligoDT were enriched from the total RNA by magnetic beads, and the captured mRNAs were fragmented, then reverse transcriptase was used to synthesize the strands of cDNA and repair the end of the transcripts product. An “A” base was added to the 3′ end of the product and the sequencing adapters were connected to the ends. The incomplete ligation products and empty linker self-ligation products were removed by ligation products purification, and the primers complementary to the linker sequence were used for PCR amplification. Finally, the sequencing library was purified by magnetic beads. Sequencing was performed using an Illumina NovaSeq 6000 system following Illumina-provided protocols for 2×150 paired-end sequencing. Bioinformatic analysis on raw sequencing data was analyzed for quality, sequence alignment and functional analysis of differentially expressed genes. Read quality control was performed for all samples using FastQC (version 0.11.9) (Andrews.2010) software. FIG. 6 depicts the fold change relative to vehicle (control) for the differentially expressed genes in both the MC38 and 3LL tumors. The following table summarizes the genes examined in this experiment demonstrating at least a two-fold increase in expression over the vehicle control.

TABLE 2
Genes Evaluated in Example 2

| Gene | Description |
|---|---|
| Batf2 | Batf2 is an activation marker gene for M1 involved in gene regulation of IFN-γ- |
| | activated classical macrophages, belongs to the leucine zipper transcription factor |
| | family. |
| CD274 | Programmed dealth-ligand 1 (PD-L1) is a 40 kDa type 1 transmembrane protein |
| (PD-L1) | that has been speculated to play a major role in suppressing the adaptive arm of |
| | immune systems. |
| Dnase1L3 | DNASE1L3 (DNASE1l3) is positively related to immune cell infiltration in many |
| | cancers. Prognostic Biomarker Associated with Immune Cell Infiltration in cancer. |
| Gbp2 | Interferon-induced guanylate-binding protein 2 is a gene related to the superfamily |
| | of large GTPases which can be induced mainly by interferon gamma. |
| IFNG | IFNG (INF-γ) has historically been considred a central player in antitumor |
| | immunity. It performs its antitumor functions through influencing both the tumor |
| | cells and the immune effector cells. |
| IL-18bp | IL-18BP is a secreted immune checkpoint and barrier to IL-18 immunotherapy. |
| IL24 | IL-24 is a potential anti-tumor agent and affects a broad array of cancers, which |
| | selectively inhibits tumor cell growth, invatin, metastasis, and angiogenesis, |
| | induces cancer-selective apoptosis, stimulates anti-cancer immune response, and |
| | sensitizes cancer cells to therapies. |
| Lag3 | Lymphocytes Activation Gene 3 (LAG3) or CD223 is expressed on multiple cell |
| | types including CD4+ and CD8+ T cells, and Tregs, and is required for optimal T |
| | cell regulation and homeostasis. Persistent antigen-stimulation in cancer or chronic |
| | infection leads to chronic LAG3 expression, promoting T cell exhaustion. |
| Pdcd1Lg2 | Programmed cell death 1 legand 2 (PDCD1LG2) is an immune checkpoint receptor |
| (PD-L2) | lgand which plays a roe in negative regulation fo the adaptive immune response |
| | PD-L2 is one of two known ligands for Programmed cell death protein 1 (PD-1). |
| Tgtp1 | T cell specific GTPase 1 (TCGTP1) was upregulated in T cells infiltrating human |
| | lung adenocarcinoma; and TCGTP1-expressing T cells exhibited greater cytokine |
| | production and cytotoxicity against lung cancer cells in vitro. |
| Tnfsf10 | Interaction of TNFSF10 with its receptors recruits the adaptor molecule FADD, |
| | activates caspases, and degrades cellular components, ultimately leading to |
| | apoposis of cancer cells. Also, evidence suggests a significant role for |
| | TNFSF10/TRAIL in regulating antitumor immunity in cancer cells and the tumor |
| | microenvironment (TME). |

It is to be understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method of treating cancer in a subject having a tumor,
comprising administering to the subject a combination comprising an MLL1-WDR5 protein-protein interaction inhibitor compound of Formula I, or a pharmaceutically acceptable salt thereof, and a PD-1 inhibitor, or a pharmaceutically acceptable salt thereof, wherein the compound of Formula I has a structure: 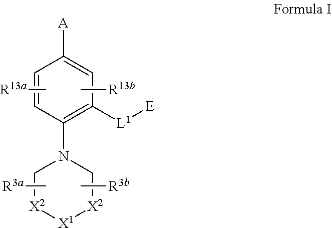
wherein:
A is 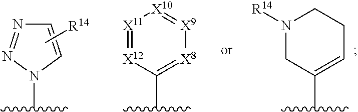
E is 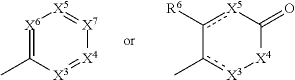
each [symbol] bond is independently a single or double bond;
L¹ is —NH— or —NH—C(═O)—;
each of R^{3a}, R^{3b}, R^{13a}, R^{13b} is independently hydrogen or a substituent;
X¹ is O, NR¹, or CR^{1a}R^{1b} wherein R¹ is H, C₁-C₆ alkyl, or C₃-C₆ cycloalkyl;
X² is a bond, —CHR^{2a}—, —CHR^{2b}CH₂—, or —CH₂CHR^{2c}—, wherein each of R^{2a}, R^{2b}, and R^{2c} is H, C₁-C₆ alkyl, or C₃-C₆ cycloalkyl;
X³ is N or CR³, or, when the [symbol] bond attached to X³ is a single bond, X³ may also be NR³, wherein R³ is H or a substituent;
X⁴ is N or CR⁴, wherein R⁴ is H or a substituent;
X⁵ is N or CR⁵, or, when the [symbol] bond attached to X⁵ is a single bond, X⁵ may also be NR⁵, wherein R⁵ is H or a substituent;
X⁶ is N or CR⁶, wherein R⁶ is H or a substituent;
X⁷ is N or CR⁷, wherein R⁷ is H or a substituent;
X⁸ is N or CR⁸, wherein R⁸ is H or a substituent;
X⁹ is N or CR⁹, wherein R⁹ is H or a substituent;
X¹⁰ is N or CR¹⁰, wherein R¹⁰ is H or a substituent;
X¹¹ is N or CR¹¹, wherein R¹¹ is H or a substituent;
X¹² is N or CR¹², where R¹² is H or a substituent;
each of R^{13a} and R^{13b} is independently H or a substituent; and
R¹⁴ is H or a substituent.

2. A method of increasing one or more anti-tumor T cell types in a subject having a tumor, comprising administering to the subject a combination comprising a compound of Formula I, or a pharmaceutically acceptable salt thereof, and a PD-1 inhibitor, or a pharmaceutically acceptable salt thereof, wherein the compound of Formula I has the structure: 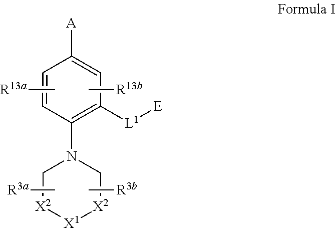
wherein:
A is 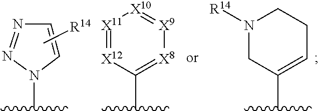
E is 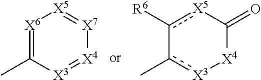
each [symbol] bond is independently a single or double bond;
L¹ is —NH— or —NH—C(═O)—;
each of R^{3a}, R^{3b}, R^{13a}, R^{13b} is independently hydrogen or a substituent;
X¹ is O, NR¹, or CR^{1a}R^{1b} wherein R¹ is H, C₁-C₆ alkyl, or C₃-C₆ cycloalkyl;
X² is a bond, —CHR^{2a}—, —CHR^{2b}CH₂—, or —CH₂CHR^{2c}—, wherein each of R^{2a}, R^{2b}, and R^{2c} is H, C₁-C₆ alkyl, or C₃-C₆ cycloalkyl;
X³ is N or CR³, or, when the [symbol] bond attached to X³ is a single bond, X³ may also be NR³, wherein R³ is H or a substituent;
X⁴ is N or CR⁴, wherein R⁴ is H or a substituent;
X⁵ is N or CR⁵, or, when the [symbol] bond attached to X⁵ is a single bond, X⁵ may also be NR⁵, wherein R⁵ is H or a substituent;
X⁶ is N or CR⁶, wherein R⁶ is H or a substituent;
X⁷ is N or CR⁷, wherein R⁷ is H or a substituent;
X⁸ is N or CR⁸, wherein R⁸ is H or a substituent;
X⁹ is N or CR⁹, wherein R⁹ is H or a substituent;
X¹⁰ is N or CR¹⁰, wherein R¹⁰ is H or a substituent;
X¹¹ is N or CR¹¹, wherein R¹¹ is H or a substituent;
X¹² is N or CR¹², where R¹² is H or a substituent;
each of R^{13a} and R^{13b} is independently H or a substituent; and
R¹⁴ is H or a substituent.

3. The method of claim 2, whereby at least one anti-tumor T cell type is increased in a tumor microenvironment of the tumor in the subject.

4. A method of inducing at least a two-fold increase in expression of at least one gene associated with anti-tumor T cell infiltration in a tumor in a subject having a tumor, comprising administering to the subject a combination comprising a compound of Formula I, or a pharmaceutically acceptable salt thereof, and a PD-1 inhibitor, or a pharmaceutically acceptable salt thereof, wherein the compound of Formula I has the structure: 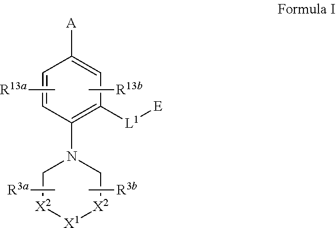
wherein:
A is 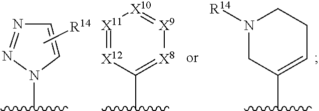
E is 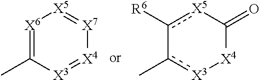
each [symbol] bond is independently a single or double bond;
L¹ is —NH— or —NH—C(═O)—;
each of R^{3a}, R^{3b}, R^{13a}, R^{13b} is independently hydrogen or a substituent;
X¹ is O, NR¹, or CR^{1a}R^{1b} wherein R¹ is H, C₁-C₆ alkyl, or C₃-C₆ cycloalkyl;
X² is a bond, —CHR^{2a}—, —CHR^{2b}CH₂—, or —CH₂CHR²—, wherein each of R^{2a}, R^{2b}, and R^{2c} is H, C₁-C₆ alkyl, or C₃-C₆ cycloalkyl; X³ is N or CR³, or, when the [symbol] bond attached to X³ is a single bond, X³ may also be NR³, wherein R³ is H or a substituent;
X⁴ is N or CR⁴, wherein R⁴ is H or a substituent;
X⁵ is N or CR⁵, or, when the [symbol] bond attached to X⁵ is a single bond, X⁵ may also be NR, wherein R⁵ is H or a substituent;
X⁶ is N or CR⁶, wherein R⁶ is H or a substituent;
X⁷ is N or CR⁷, wherein R⁷ is H or a substituent;
X⁸ is N or CR⁸, wherein R⁸ is H or a substituent;
X⁹ is N or CR⁹, wherein R⁹ is H or a substituent;
X¹⁰ is N or CR¹⁰, wherein R¹⁰ is H or a substituent;
X¹¹ is N or CR¹¹, wherein R¹¹ is H or a substituent;
X¹² is N or CR¹², where R¹² is H or a substituent;
each of R^{13a} and R^{13b} is independently H or a substituent; and
R¹⁴ is H or a substituent.

5. The method of claim 4, wherein the gene associated with an anti-tumor T cell is Batf2, Cd274 (PD-L1), Dnase1L3, Gbp2, Infg (Inf-γ), IL18 bp, IL24, Lag3, Pdcdl1g2 (PD-L2), Tgtp1, Tnfsf10, or a combination of any two or more thereof.

6. The method of claim 1, wherein the PD-1 inhibitor is an anti-PD-1 antibody.

7. The method of claim 2, wherein the one or more T cell types comprise one or more of tumor-infiltrating T lymphocytes (TILs) and cytotoxic T lymphocytes (CTLs).

8. The method of claim 2, wherein a ratio of tumor infiltrating leukocytes (TIL) to regulatory T cells (Tregs) in the tumor is increased.

9. The method of claim 1, wherein one or more of regulatory macrophages (M2), tumor-associated myeloid cells (TAMCs), myeloid-derived suppressor cells (M-MDSCs) and polymorphonuclear myeloid-derived suppressor cells (PMN-MDSCs) are reduced in the tuner--subject.

10. The method of claim 9, wherein the decrease in one or more of M2, TAMCs, M-MDSCs, and PMN-MDSCs is measured relative to a baseline obtained from a biopsy taken at or prior to commencement of treatment with the combination.

11. The method of claim 1, wherein A is: 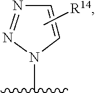
wherein R¹⁴ is a substituent with the structure: 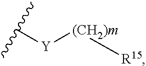
wherein Y is absent, —O—, —S—, —C(O)—, —CH₂O—, —(CO)O—, —O(CO)—, —NR^{15h}—, —C(O)NR^{15g}, or —NR^{15h}C(0);
m is 0 to 6;
R¹⁵ is hydrogen, amino, hydroxyl, thiol, carboxyl, cyano, C₁-C₄ alkyl, substituted C₁-C₄ alkyl, C₁-C₆ alkoxy, substituted or unsubstituted phenyl, substituted or unsubstituted C₃-C₆ cycloalkyl, substituted or unsubstituted nitrogen- or oxygen-containing 3 to 7 membered heterocyclic ring, or substituted or unsubstituted 3 to 7 membered heterocyclic ring containing both nitrogen and oxygen, wherein substituents on the heterocyclic ring can optionally be on the hetero atom; unsaturated heterocycloalkyl containing nitrogen or oxygen or both, —NR^{15a}COR^{15b} —OR¹⁵c°, —C(O)O—R^{15c} —O(CO)O—R^{15c} —C(O)NR^{15d}R^{15e} or —NR^{15d}R^{15e}; wherein
R^{15a} is hydrogen, C₁-C₄ alkyl, C₁-C₄ haloalkyl, substituted or unsubstituted phenyl,
R^{15b} is hydrogen, amino, hydroxyl, C₁-C₄ alkyl, C₁-C₄ alkoxy, substituted or unsubstituted phenyl, substituted or unsubstituted nitrogen- or oxygen-containing 3 to 7 membered heterocyclic ring, wherein substituents on the heterocyclic ring can be on the hetero atom,
R^{15d} and R^{15e} are each independently hydrogen, C₁-C₄ alkyl, substituted or unsubstituted phenyl, substituted or unsubstituted C₃-C₇ cycloalkyl, substituted or unsubstituted nitrogen- or oxygen-or both containing 3 to 7 membered heterocyclic ring, wherein the heterocyclic ring may be aryl, partially unsaturated, or fully saturated, wherein substituents on the heterocyclic ring can be on the hetero atom, or
R^{15c} is C₁-C₄ alkyl, C₁-C₄ haloalkyl, substituted or unsubstituted phenyl, substituted or unsubstituted C₃-C₇ cycloalkyl, substituted or unsubstituted nitrogen- or oxygen- or both containing 3 to 7 membered heterocyclic ring; wherein substituents on the heterocyclic ring can be on the hetero atom;
R^{15d} and R^{15c} together form: a nitrogen-, oxygen-, or nitrogen and oxygen, or nitrogen and nitrogen or oxygen and oxygen containing 3 to 7 membered heterocyclic ring, wherein the heterocyclic ring is optionally substituted with one, two or three substituents which are independently halogen, C₁-C₄ alkyl, C₁-C₄ alkoxy, amino, hydroxyl, thiol, carboxyl, cyano, trifluoromethyl or imidazolyl; wherein substituents on the heterocyclic ring can be on the hetero atom;
R^{15f}, R¹ and R^{15h} each independently represents hydrogen, C₁-C₄ alkyl, C₁-C₄ haloalkyl, or substituted or unsubstituted phenyl, wherein the phenyl is substituted with one, two or three of halogen, amino, cyano, hydroxyl, trifluoro C₁-C₄ alkyl, C₁-C₄ alkoxy, carboxyl, or imidazolyl.

12. The method of claim 1, wherein A is:

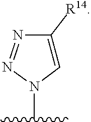

13. The method of claim 11, wherein Y is absent.

14. The method of claim 13, wherein Y is —O—, —S—, —C(O)—, —CH₂O—, —(CO)O—, —O(CO)—, —NR¹⁵1, —C(O)NR^{15g}—, or NR^{15h}C(O).

15. The method of claim 13, wherein Y is —O—, —NR¹⁵—, or —C(O)NR^{15g}—, wherein R^{15f} and R^{15g} are independently hydrogen or C₁-C₄ alkyl.

16. The method of claim 1, wherein A is: 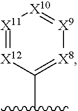
wherein: each of R⁸, R⁹, R¹⁰, R¹¹, and R¹², when present, is independently selected from H, C₁-C₆ alkyl; substituted C₁-C₆ alkyl (wherein each substituent is a 3-7-membered cycloalkyl, a 3-7-membered heterocyclic ring containing nitrogen or oxygen, or a 3-7-membered heterocyclic ring containing both nitrogen and oxygen); unsubstituted, mono-substituted, di-substituted or tri-substituted 3-7-membered heterocyclic ring containing nitrogen, oxygen or both (wherein the heterocyclic ring is aromatic, partially unsaturated or fully saturated and each substituent is independently C₁-C₆ alkyl, or NR^{D}R^{E}); —NHCOR^{E}; —CONR^{D}R^{E}; —COR^{F}; or —OR^{G}; wherein:
R^{E} is: hydrogen, C₁-C₆ alkyl, 3-7-membered cycloalkyl, 3-7-membered heterocycloalkyl containing nitrogen, oxygen or both, or substituted C₁-C₆ alkyl (wherein the substituent is 3-7-membered cycloalkyl, 3-7-membered heterocyclic ring containing nitrogen or oxygen, or 3-7-membered heterocyclic ring containing both nitrogen and oxygen);
R^{D}, R^{E} each independently is: hydrogen, C₁-C₆ alkyl, phenyl or substituted phenyl, substituted or unsubstituted 3-7-membered heterocyclic ring containing nitrogen, oxygen or both; substituted C₁-C₆ alkyl (wherein each substituent on R^{D} and R^{E} is independently a 3-7-membered cycloalkyl, a 3-7-membered heterocyclic ring containing nitrogen or oxygen, or a 3-7-membered heterocyclic ring containing both nitrogen and oxygen);
R^{F} is: C₁-C₆ alkyl, C₁-C₆ substituted alkyl (wherein the substituent is C₃-C₆ cycloalkyl), or NR^{D}R^{E} (wherein R^{D} and R^{E} are defined in the immediate foregoing paragraph);
R^{G} is: C₁-C₆ alkyl, C₁-C₆ substituted alkyl (wherein the substituent is C₃-C₆ cycloalkyl).

17. The method of claim 1, wherein A is: 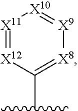
wherein:
X⁹ is N,
X¹¹ is N,
X⁸ is CR⁸, wherein R⁸ is H or a substituent,
X¹² is CR¹², wherein R¹² is H or a substituent,
X¹⁰ is CR¹⁰, wherein R¹⁰ is H or a substituent.

18. The method of claim 1, wherein A is: 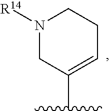
wherein R¹⁴ is a substituted 5-6-membered aromatic heterocyclic ring containing oxygen, nitrogen or both, wherein each heterocyclic ring substituent is independently C₁-C₄ alkyl, substituted C₁-C₄ alkyl (wherein the alkyl substituent is —NR^{14a}R^{14b}, wherein R^{14a} and R^{14b} are independently C₁-C₄ alkyl or 3-7-membered heterocyclic ring containing nitrogen or oxygen or both, or R^{14a} and R^{14b} are linked together to form a 3-7-membered heterocyclic ring containing nitrogen, oxygen or both).

19. The method of claim 1, wherein E is: 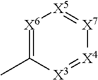
wherein each R³, R⁴, R⁵, R⁶, R⁷, when present, is independently hydrogen, halogen, amino, cyano, hydroxy, thiol, nitro, C₁-C₆ alkyl, C₃-C₇ cycloalkyl, C₁-C₆ alkoxy, C₃-C₇ cycloalkoxy, trifluoromethyl, difluoromethyl, trifluoromethoxy, difluoromethoxy, C₁-C₆ alkylthio, C₁-C₆ alkylsulfinyl, C₁-C₆ alkylsulfonyl, hydroxy-(C₁-C₆)alkyl, amino-(C₁-C₆)alkyl, or —C(O)NR^{H}R, wherein R^{H} and R¹ are each independently hydrogen or C₁-C₆ alkyl.

20. The method of claim 1, wherein E is: 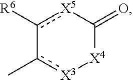
wherein each R³, R⁴, and R⁵ when present, is independently hydrogen, halogen, amino, cyano, hydroxy, thiol, nitro, C₁-C₆ alkyl, C₃-C₇ cycloalkyl, C₁-C₆ alkoxy, C₃-C₇ cycloalkoxy, trifluoromethyl, difluoromethyl, trifluoromethoxy, difluoromethoxy, C₁-C₆ alkylthio, C₁-C₆ alkylsulfinyl, C₁-C₆ alkylsulfonyl, hydroxy-(C₁-C₆)alkyl, amino-(C₁-C₆)alkyl, or —C(O)NR^{H}R, wherein R^{H} and R¹ are each independently hydrogen or C₁-C₆ alkyl.

21. The method of claim 1, wherein L¹ is —NH—C(═O)—.

22. The method of claim 1, wherein L¹ is —NH—.

23. The method of claim 1, wherein:
R^{3a} is hydrogen, C₁-C₆ alkyl, or C₃-C₆ cycloalkyl;
R^{3b} is hydrogen, C₁-C₆ alkyl, or C₃-C₆ cycloalkyl;
R^{13a} is hydrogen, halogen, methyl, methoxy, difluoromethoxy, or trifluoromethoxy;
R^{13b} is hydrogen, halogen, methyl, methoxy, difluoromethoxy, or trifluoromethoxy;
X¹ is NR¹, wherein R¹ is hydrogen, C₁-C₆ alkyl, or C₃-C₆ cycloalkyl;
X² is —CHR^{2a}—, —CHR^{2b}CH₂—, or —CH₂CHR^{2c}—, wherein each of R^{2a}, R^{2b}, and R², is hydrogen, C₁-C₆ alkyl, or C₃-C₆ cycloalkyl;
each of R⁴, R⁵, R⁶, and R⁷ is independently hydrogen, halogen, amino, cyano, hydroxy, thiol, nitro, C₁-C₆ alkyl, C₃-C₇ cycloalkyl, C₁-C₆ alkoxy, C₃-C₇ cycloalkoxy, trifluoromethyl, difluoromethyl, trifluoromethoxy, difluoromethoxy, C₁-C₆ alkylthio, C₁-C₆ alkylsulfinyl, C₁-C₆ alkylsulfonyl, hydroxy-(C₁-C₆)alkyl, amino-(C₁-C₆)alkyl, or —C(O)NR^{A}R^{B}, wherein R^{A} and R^{B} are each independently hydrogen or C₁-C₆ alkyl.

24. The method claim 1, wherein:
X³ is CR³, wherein R³ is H or a substituent;
X⁴ is CR⁴, wherein R⁴ is H or a substituent;
X⁵ is CR⁵, wherein R⁵ is H or a substituent.

25. The method of claim 24, wherein E is: 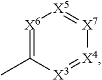
X⁶ is CR⁶, wherein R⁶ is H or a substituent; and
X⁷ is CR⁷, wherein R⁷ is H or a substituent.

26. The method of claim 24, wherein E is: 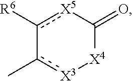
and R⁶ is H or a substituent.

27. The method of claim 1, wherein:
X³ is N;
X⁴ is CR⁴, wherein R⁴ is H or a substituent; and
X⁵ is CR⁵, wherein R⁵ is H or a substituent.

28. The method of claim 27, wherein E is: 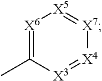
X⁶ is CR⁶, wherein R⁶ is H or a substituent;
X⁷ is CR⁷, wherein R⁷ is H or a substituent.

29. The method of claim 27, wherein E is: 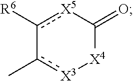
and R⁶ is H or a substituent.

30. The method of claim 1, wherein X³ is CR³, wherein R³ is H or a substituent;
X⁴ is N; and
X⁵ is CR⁵, wherein R⁵ is H or a substituent.

31. The method of claim 30, wherein E is: 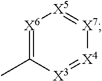
X⁶ is CR⁶, wherein R⁶ is H or a substituent; and
X⁷ is CR⁷, wherein R⁷ is H or a substituent.

32. The method of claim 30, wherein E is: 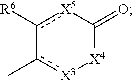
and R⁶ is H or a substituent.

33. The method of claim 1, wherein
X³ is CR³, wherein R³ is H or a substituent;
X⁴ is CR⁴, wherein R⁴ is H or a substituent; and
X⁵ is N.

34. The method of claim 33, wherein E is: 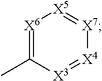
X⁶ is CR⁶, wherein R⁶ is H or a substituent; and
X⁷ is CR⁷, wherein R⁷ is H or a substituent.

35. The method of claim 34, wherein E is: 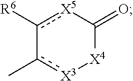
and R⁶ is H or a substituent.

36. The method of claim 1, wherein X³ and X⁴ are N; and
X⁵ is CR⁵, wherein R⁵ is H or a substituent.

37. The method of claim 36, wherein E is: 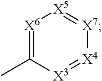
X⁶ is CR, wherein R⁶ is H or a substituent; and
X⁷ is CR⁷, wherein R⁷ is H or a substituent.

38. The method of claim 36, wherein E is: 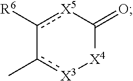
and R⁶ is H or a substituent.

39. The method of claim 1, wherein E is: 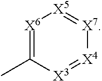
X³ is CR³, wherein R³ is H or a substituent;
X⁴ is CR⁴, wherein R⁴ is H or a substituent;
X⁵ is CR⁵, wherein R⁵ is H or a substituent; and
X⁶ and X⁷ are N.

40. The method of claim 1, wherein one, two, or
three of X³, X⁴, and X⁵ are N.

41. The method of claim 1, wherein at least one of X³, X⁴, and X⁵ is N.

42. The method of claim 41, wherein only one of X³, X⁴, and X⁵ is N.

43. The method of claim 39, wherein X⁶ is CR⁶ and X⁷ is CR⁷.

44. The method of claim 42, wherein X³ is CR³, X⁴ is N, and X⁵ is CR⁵.

45. The method of claim 1, wherein each of R³, R⁴, R⁵, R⁶, and R⁷ is hydrogen, halogen, amino, cyano, hydroxy, thiol, nitro, C₁-C₆ alkyl, C₃-C₇ cycloalkyl, C₁-C₆ alkoxy, C₃-C₇ cycloalkoxy, trifluoromethyl, difluoromethyl, trifluoromethoxy, difluoromethoxy, C₁-C₆ alkylthio, C₁-C₆ alkylsulfinyl, C₁-C₆ alkylsulfonyl, hydroxy-(C₁-C₆)alkyl, amino-(C₁-C₆)alkyl, or —C(O)NR^{A}R^{B}, wherein R^{A} and R^{B} are each independently hydrogen or C₁-C₆ alkyl.

46. The method of claim 1, wherein each of R³, R⁵, R⁶ and R⁷ is independently hydrogen, halogen, C₁-C₆ alkyl, C₃-C₇ cycloalkyl, C₁-C₆ alkoxy, C₃-C₇ cycloalkoxy, trifluoromethyl, difluoromethyl, trifluoromethoxy, difluoromethoxy, C₁-C₆ alkylthio, C₁-C₆ alkylsulfinyl, C₁-C₆ alkylsulfonyl, nitro or cyano.

47. The method of claim 1, wherein E is: 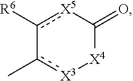
wherein
each of R³, R⁴, R⁵, R⁶, and R⁷ is hydrogen, halogen, cyano, nitro, C₁-C₆ alkyl, C₃-C₇ cycloalkyl, C₁-C₆ alkoxy, C₃-C₇ cycloalkoxy, trifluoromethyl, difluoromethyl, trifluoromethoxy, difluoromethoxy, C₁-C₆ alkylthio, C₁-C₆ alkylsulfinyl, or C₁-C₆ alkylsulfonyl.

48. The method of claim 47, wherein X³ is NH.

49. The method of claim 1, wherein the compound of Formula I is selected from:

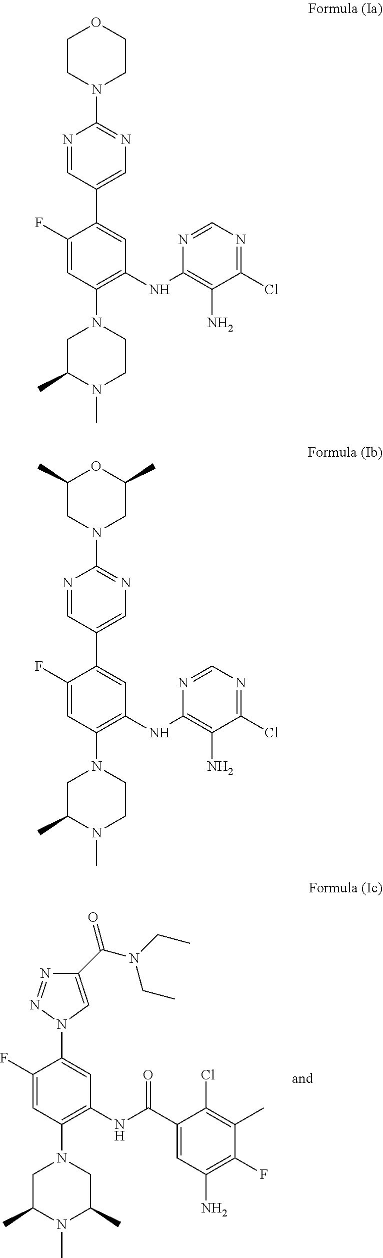

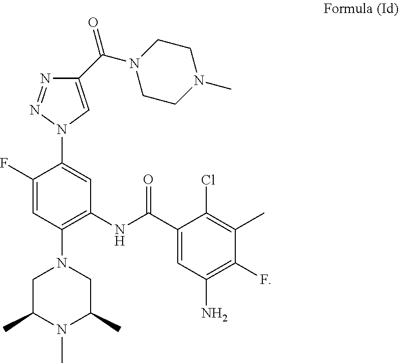 [SMILES: Cc1c(F)c(N)cc(C(=O)Nc2cc(-n3cc(C(=O)N4CCN(C)CC4)nn3)c(F)cc2N2C[C@@H](C)N(C)[C@@H](C)C2)c1Cl]

50. The method of claim 49, wherein the compound of Formula I is Formula (Id)

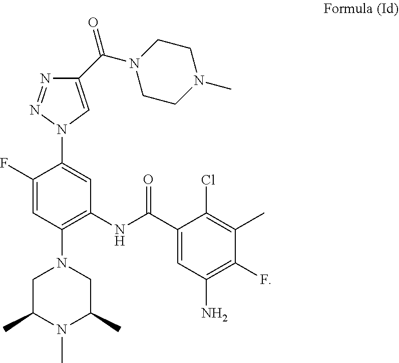 [SMILES: Cc1c(F)c(N)cc(C(=O)Nc2cc(-n3cc(C(=O)N4CCN(C)CC4)nn3)c(F)cc2N2C[C@@H](C)N(C)[C@@H](C)C2)c1Cl]

51. The method of claim 1, wherein:
a. the compound of Formula I is formulated in a first pharmaceutical composition comprising the compound of Formula I, or a pharmaceutically acceptable salt, and pharmaceutically acceptable excipient; and
b. the PD-1 inhibitor is formulated in a second pharmaceutical composition comprising the PD-1 inhibitor, or a pharmaceutically acceptable salt thereof, and pharmaceutically acceptable excipient.

52. The method of claim 1, wherein the subject has received one or more previous treatments for the cancer.

53. The method of claim 52, wherein the subject has previously received immunotherapy.

54. The method of claim 53, wherein the subject has previously received immunotherapy with one or more checkpoint inhibitors.

55. The method of claim 54, wherein at least one of the one or more checkpoint inhibitors is selected from one or more PD-1 inhibitors, one or more PD-L1 inhibitors, one or more CTLA-4 inhibitors, and combinations of two or more thereof.

56. (canceled)

57. A process of making the combination recited in claim 1, wherein the combination is for use in the treatment of cancer in a subject in need of such treatment.

58. The method of claim 2, wherein the compound of Formula I is selected from:

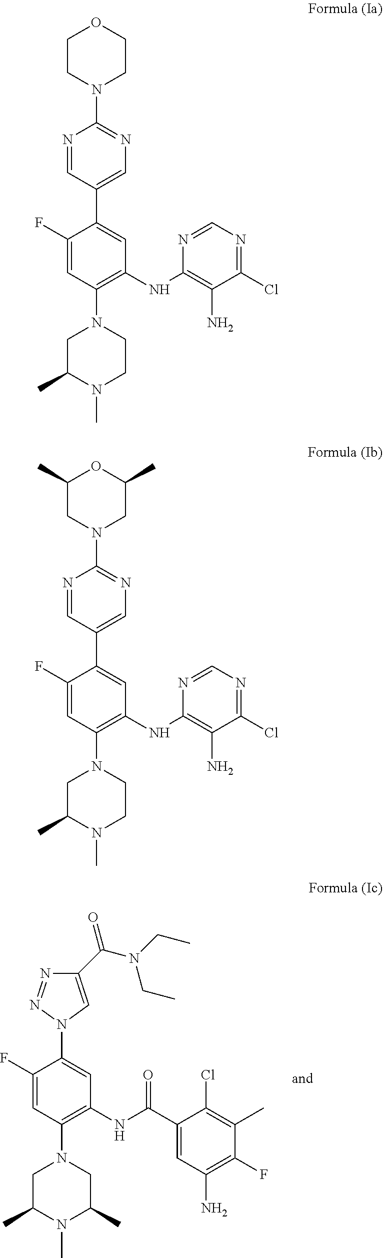

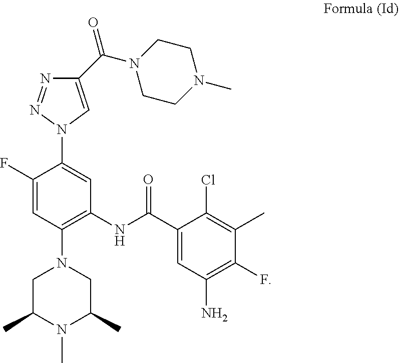 [SMILES: Cc1c(F)c(N)cc(C(=O)Nc2cc(-n3cc(C(=O)N4CCN(C)CC4)nn3)c(F)cc2N2C[C@@H](C)N(C)[C@@H](C)C2)c1Cl]

59. The method of claim 4, wherein the compound of Formula I is selected from: 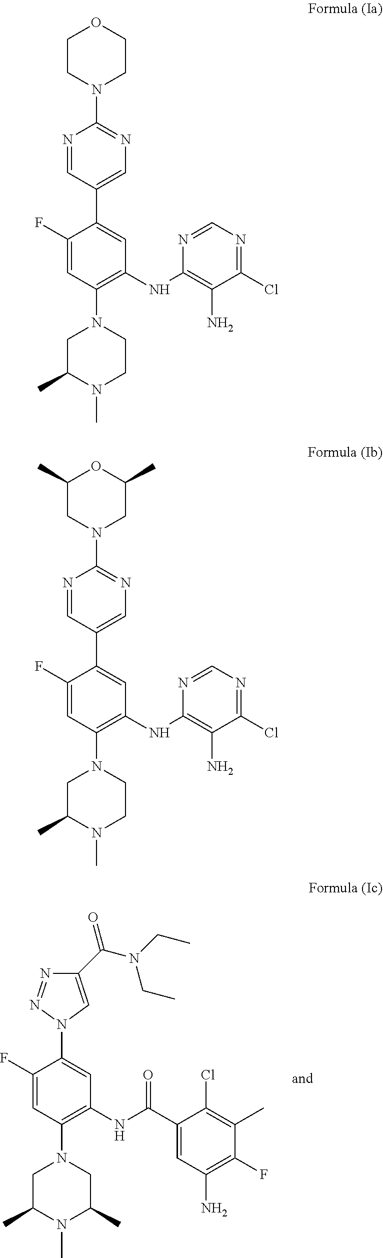 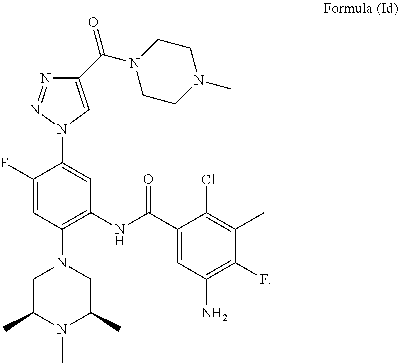 [SMILES: Cc1c(F)c(N)cc(C(=O)Nc2cc(-n3cc(C(=O)N4CCN(C)CC4)nn3)c(F)cc2N2C[C@@H](C)N(C)[C@@H](C)C2)c1Cl]
